(19) United States
(12) Patent Application Publication
(10) Pub. No.: US 2023/0365493 A1
(43) Pub. Date: Nov. 16, 2023

(54) COMPOUNDS AND METHODS

(71) Applicant: DEMAGTECH PTY LTD, Perth, Western Australia (AU)

(72) Inventors: Joshua Scott MYLNE, Nedlands, Western Australia (AU); Keith STUBBS, Nedlands, Western Australia (AU); Joel HAYWOOD, Nedlands, Western Australia (AU); Karen BREESE, Nedlands, Western Australia (AU)

(21) Appl. No.: 18/024,720

(22) PCT Filed: Sep. 8, 2021
(86) PCT No.: PCT/AU2021/051039

§ 371 Date: Mar. 3, 2023

(30) Foreign Application Priority Data
Sep. 8, 2020 (AU) 2020903207
Sep. 8, 2020 (AU) 2020903208

(51) Int. Cl. C07C 229/38; C07C 229/52; C07C 327/30; C07D 207/08; C07D 213/46; A01N 37/40; A01N 37/44; A01N 43/40; C07D 295/145; A01N 43/84; A01N 43/36

(52) U.S. Cl. CPC C07C 229/38; C07C 229/52; C07C 327/30; C07D 207/08; C07D 213/46; A01N 37/40; A01N 37/44; A01N 43/40; C07D 295/145; A01N 43/84; A01N 43/36

(57) ABSTRACT

Compounds of Formula (A) and methods for use as agricultural chemicals, including herbicides.

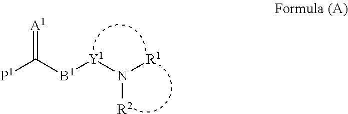

This application claims priority from Australian provisional patent applications 2020903207 and 2020903208, the contents of which are incorporated by reference.

### FIELD OF THE INVENTION

The present invention relates to novel compounds, which may have agrichemical activity such as herbicides, insecticides, fungicides and plant growth regulators; and to methods for the use of said compounds. The agrichemical compounds of the present invention may have beneficial volatility properties that reduce the extent of drift of the compounds from target areas to non-target areas.

In one form, the present invention relates to compounds having herbicidal activity, and methods for the use of said compounds for controlling the growth of auxin-susceptible plants.

### BACKGROUND ART

An extensive range of chemicals are used in agriculture. For example, the protection of crops from pests that impact crop yields is a recurring problem in agriculture. Researchers have produced an extensive variety of pesticides and pesticidal formulations effective in the control of such pests. As a further example, plant growth regulators such as daminozide are used to modify growth and ripening behaviours to optimise harvests.

However, many such agrichemicals are volatile and suffer from drift. Localised application of agrichemical compounds is highly desirable. For example, unintentional application of herbicides to a sensitive plant generally causes severe injury, loss of yield, and even death of the non-target plants, and unintentional application of insecticides and fungicides are associated with undesirable environmental impact.

For example, the protection of crops from weeds that inhibit crop growth is a recurring problem in agriculture. Researchers have produced an extensive variety of herbicides and herbicidal formulations effective in the control of such unwanted weeds.

Synthetic auxin herbicides are one such class of herbicides. Auxin herbicides kill auxin-susceptible plants by inducing hormonal effects that promote unsustainable growth. Dicamba (3,6-dichloro-2-methoxybenzoic acid) is an auxin herbicide. Dicamba has been in use since the 1960s. However, it was traditionally used sparingly, and not on some growing crops as they were susceptible to damage and additionally the free-acid form of dicamba is volatile and suffers from drift.

The term drift can be used to describe two distinct phenomena. Spray drift is the movement of pesticide dust or droplets through the air at the time of application or soon thereafter, to any site other than the area intended. The term drift is also used to describe post-application movement arising from volatilisation of the active agent in a herbicidal composition. Volatilization occurs when pesticide surface residues change from a solid or liquid to a vapor after application. Once airborne, volatile pesticides can move long distances off site (longer distances compared to spray drift).

Non-target plant damage associated with auxin herbicide volatilization is a major concern for crop growers. Unintentional application of auxin herbicides to a sensitive plant generally causes severe injury, loss of yield, and even death of the non-target plants.

An object of the present invention is to provide compounds with agrichemical activity and reduced volatility relative to at least some currently available agrichemical compounds, or to at least provide a useful alternative thereto.

An object of certain embodiments of the of the present invention is to provide novel compounds with herbicidal activity and reduced volatility relative to at least some currently available auxin herbicides, or to at least provide a useful alternative thereto.

The discussion of the background art is included exclusively for the purpose of providing a context for the present invention. It is not an acknowledgement or admission that any of the material referred to was common general knowledge in the field relevant to the present invention in Australia or elsewhere before the priority date.

### DISCLOSURE OF THE INVENTION

In one aspect, the invention comprises a compound of Formula (A), as defined in the following detailed description of the invention.

In a preferred form of the first aspect of the invention, the invention comprises a compound of Formula (A1) as defined in the following detailed description of the invention.

In a second aspect, the invention comprises compositions comprising an agriculturally effective amount of a compound of Formula (B), as defined in the following detailed description of the invention, a salt or solvate thereof, and an agriculturally acceptable adjuvant or carrier. Examples of the second aspect of the invention are herbicidal compositions comprising a herbicidally effective amount of a compound of Formula (B) and an agriculturally acceptable adjuvant or carrier.

In a preferred form of the second aspect of the invention, the invention comprises a herbicidal composition comprising a herbicidally-effective amount of a compound of Formula (1) as defined in the following detailed description of the invention, or salts and solvates thereof, and an agriculturally acceptable adjuvant or carrier:

In another aspect, the invention comprises a method for suppressing the growth of an auxin-susceptible plant, the method comprising applying to the plant a herbicidally effective amount of a compound of Formula (1), or salts or solvates thereof.

In another aspect, the invention comprises a method for suppressing the growth of an auxin-susceptible plant, the method comprising applying to the plant a herbicidal composition comprising a herbicidally-effective amount of a compound of Formula (1), or salts and solvates thereof, and an agriculturally acceptable adjuvant or carrier:

### DETAILED DESCRIPTION OF THE INVENTION

Compounds

The inventors have identified a means for modifying known volatile agrichemical compounds to form new compounds of reduced volatility, and thus less prone to drift issues, while retaining activity, in some cases activity comparable to the original agrichemical compound.

The invention is readily applicable to known agrichemical compounds having at least one carboxylic acid group, thiocarboxylic acid group (both thione and thiol), or dithiocarboxylic acid group, and esters thereof.

Examples of known agrichemical compounds that are structurally related to example compounds of the invention are discussed below.

Daminozide (4-(2,2-Dimethylhydrazinyl)-4-oxobutanoic acid; also known as Alar, Kylar, B-NINE, DMASA, SADH, or B 995) is a plant growth regulator sprayed on fruit to regulate growth, make harvest easier, and keep apples from falling off the trees before they ripen so they are red and firm for storage.

Daminozide has regulatory approval for use in the EU and is also used in Australia and USA. It is highly soluble in water and volatile. Based on its chemical properties it is not expected to leach to groundwater. Data suggests it is not persistent in soil systems but can be persistent in some water systems. It is relatively non-toxic to mammals but has a high potential for bioaccumulation. Daminozide is a recognised irritant. It has a moderate toxicity to aquatic invertebrates and earthworms but is less harmful to birds, fish and honeybees.

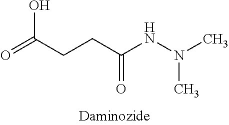

Endothall (7-oxabicyclo[2.2.1]heptane-2,3-dicarboxylic acid; as the dipotassium salt or the mono-N,N-dimethylalkylamine salt) is used as a herbicide for both terrestrial and aquatic plants. It is used as an aquatic herbicide for submerged aquatic plants and algae in lakes, ponds and irrigation canals, as a desiccant on potatoes, hops, cotton, clover and alfalfa, and a biocide to control molluscs and algae in cooling towers.

Endothal does not have regulatory approval for use in the EU. It is highly soluble in water and semi-volatile. Based on its chemical properties it is not expected to leach to groundwater. It is generally non-persistent in soils. Endothal is highly toxic to mammals but is not expected to bioaccumulate. It is a recognised irritant. It is moderately toxic to fish and aquatic organisms but is less toxic to birds.

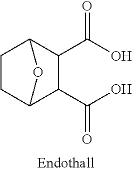 [SMILES: O=C(O)C1C2CCC(O2)C1C(=O)O]

Metalaxyl (methyl 2-[(2,6-dimethylphenyl)(methoxyacetyl)amino]propanoate) is an acylalanine fungicide with systemic function. It can be used to control diseases caused by air- and soil-borne Peronosporales in many different crops, including Pythium in vegetable crops, and Phytophthora in peas. Metalaxyl-M or Ridomil Gold are trade names for the optically pure (−)/D/R active stereoisomer, which is also known as Mefenoxam. It is the active ingredient in the seed treatment agent Apron XL LS.

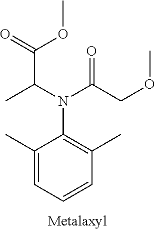

Metsulfuron (2-{[[(4-methoxy-6-methyl-1,3,5-triazin-2-yl)amino]-oxomethyl]sulfamoyl}benzoic acid methyl ester) is a sulfonylurea herbicide, which kills broadleaf weeds and some annual grasses, mainly in cereals and land temporarily removed from production. It is also be a pesticide transformation product. It has residual activity in soils, allowing it to be used infrequently but requiring up to 22 months before planting certain crops (sunflowers, flax, corn, or safflower).

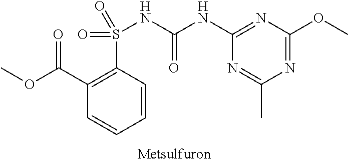

Mesosulfuron methyl (methyl 2-[[[[(4, 6-dimethoxy-2-pyrimidinyl) amino] carbonyl] amino]sulfonyl]-4-[[(methylsulfonyl) amino] methyl] benzoate) is a systemic herbicide used for post-emergence control of grasses and other weeds in cereals.

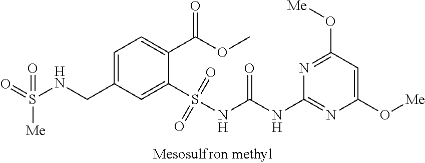 [SMILES: COC(=O)c1ccc(CNS(C)(=O)=O)cc1S(=O)(=O)NC(=O)Nc1nc(OC)cc(OC)n1]

Dicamba (3,6-dichloro-2-methoxybenzoic acid) is a broad-spectrum herbicide first registered in 1967, and sold under names including Dianat, Banvel, Diablo, Oracle and Vanquish. Dicamba is a chlorinated derivative of o-anisic acid. Dicamba's use has come under significant scrutiny due to its tendency to spread from treated fields into neighboring fields, causing damage.

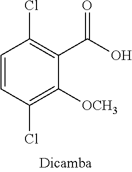 [SMILES: COc1c(Cl)ccc(Cl)c1C(=O)O]

Aminopyralid (4-amino-3,6-dichloropyridine-2-carboxylic acid) is a pyridine carboxylic acid herbicide that is currently under approval consideration for use in the EU. It is highly soluble in water and, based on its chemical properties, is mobile and has a high potential for leaching to groundwater. It may be moderately persistent in soil systems but would not be expected to persistent in surface water under normal conditions. It has a low mammalian toxicity and there is some concern regarding its potential for bioaccumulation. Aminopyralid has a low to moderate toxicity to most terrestrial and aquatic species.

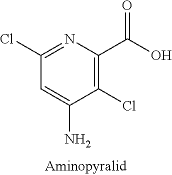 [SMILES: Nc1cc(Cl)nc(C(=O)O)c1Cl]

Clopyralid (3,6-dichloropyridine-2-carboxylic acid) is a herbicide approved for use in the EU. It has a high aqueous solubility, is volatile and, based on its chemical properties, there is a high risk of it leaching to groundwater. It can be persistent in both soil and water systems depending upon conditions. It has a low mammalian toxicity and is not expected to bioaccumulate. It is an irritant. It is moderately toxic to birds, fish, aquatic invertebrates, honeybees and earthworms. It has a low toxicity to aquatic plants and algae.

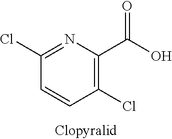 [SMILES: O=C(O)c1nc(Cl)ccc1Cl]

Fluroxypyr ([(4-amino-3,5-dichloro-6-fluoro-2-pyridinyl)oxy]acetic acid) is a post-emergence herbicide used to control economically important broad-leaved weeds.

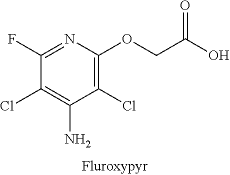 [SMILES: Nc1c(Cl)c(F)nc(OCC(=O)O)c1Cl]

Chloramben (3-amino-2,5-dichlorobenzoic acid) is an obsolete pre-emergence herbicide that is no longer approved in the EU. It is highly soluble in water, volatile, mobile and has a tendency to leach to groundwater. It is not normally persistent in soil or water systems. It has a low mammalian toxicity but a high potential for bioaccumulation. It is a recognised irritant and there are concerns that it may be a developmental/reproduction toxicant. Chloramben has a low toxicity to birds and aquatic invertebrates but is moderately toxic to fish and honeybees.

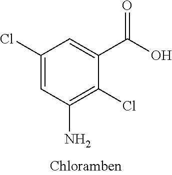 [SMILES: Nc1cc(Cl)cc(C(=O)O)c1Cl]

2,3,6-TBA (2,3,6-trichlorobenzoic acid) is a post-emergence herbicide that does not have EU approval for use. It is highly soluble in water and most organic solvents. It is considered volatile. Little has been reported on its environmental persistence or mobility. It is moderately toxic to mammals and a recognised irritant. There are gaps in knowledge regarding its toxicity to biodiversity however data has shown that it is moderately toxic to bids, fish and honeybees.

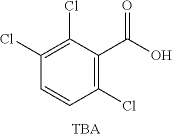 [SMILES: O=C(O)c1c(Cl)ccc(Cl)c1Cl]

Quinclorac (3,7-dichloro-8-quinolinecarboxylic acid) is a herbicide used for post-emergence weed control on grass and turf.

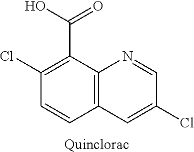

Quinmerac (7-Chloro-3-methyl-8-quinolinecarboxylic acid) is a residual herbicide used to control broad-leaved weeds on a range of crops including cereals.

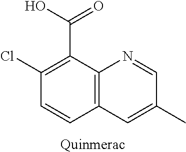

Imazamox (2-[4,5-Dihydro-4-methyl-4-(1-methylethyl)-5-oxo-1H-imidazol-2-yl]-5-(methoxymethyl)-3-pyridinecarboxylic acid) is a post-emergence herbicide used to control weeds including those in aquatic situations.

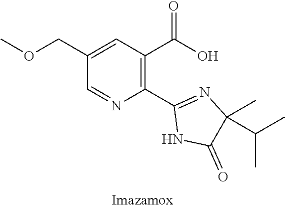 [SMILES: COCc1cnc(C2=NC(C)(C(C)C)C(=O)N2)c(C(=O)O)c1]

2,4-DB (4-(2,4-dichlorophenoxy)butanoic acid) is a post-emergence herbicide that has approval for use in the EU. It is highly soluble in water and most organic solvents. It is relatively volatile but it may leach to groundwater under certain conditions. It is not persistent in soil systems but may tend to be persistent in aquatic systems. It is moderately toxic to mammals and there is some concern regarding its potential for bioaccumulation. It is moderately toxic to birds, earthworms, honeybees and most aquatic organisms.

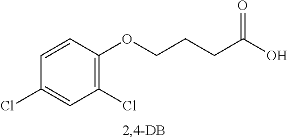 [SMILES: O=C(O)CCCOc1ccc(Cl)cc1Cl]

MCPB (4-(4-chloro-2-methylphenoxy)butanoic acid) is a herbicide for post-emergence control of annual and perennial broad-leaved weeds

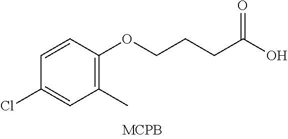

Imazapic (5-methyl-2-[4-methyl-5-oxo-4-(propan-2-yl)-4,5-dihydro-1H-imidazol-2-yl]pyridine-3-carboxylic acid) is a selective pre- and post-emergent herbicide without EU regulatory approval for use. It has a high aqueous solubility, is volatile and, based on its chemical properties, is moderately mobile and may leach to groundwater. It is may be persistent in soil systems but usually degrades quickly in aquatic systems via photolysis. It has a low mammalian toxicity and has a high potential for bioaccumulation. It has a low level of toxicity to birds but is more toxic to aquatic life and honeybees.

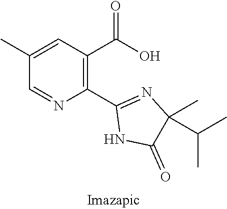 [SMILES: Cc1cnc(C2=NC(C)(C(C)C)C(=O)N2)c(C(=O)O)c1]

Imazethapyr (2-[4,5-dihydro-4-methyl-4-(1-methylethyl)-5-oxo-1H-imidazol-2-yl]-5-ethyl-3-pyridinecarboxylic acid) is a herbicide used to control a variety of broad-leaved weeds and grasses in numerous crops.

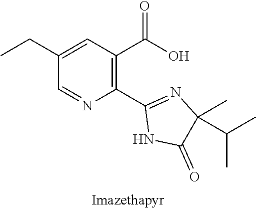 [SMILES: CCc1cnc(C2=NC(C)(C(C)C)C(=O)N2)c(C(=O)O)c1]

Imazaquin (2-[4,5-dihydro-4-methyl-4-(1-methylethyl)-5-oxo-1H-imidazol-2-yl]-3-quinolinecarboxylic acid) is used as a pre- and post-emergence herbicide particularly for the control of grasses and broad-leaved weeds.

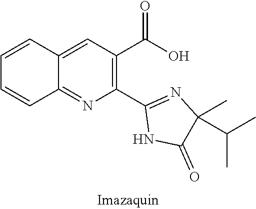 [SMILES: CC(C)C1(C)N=C(c2nc3ccccc3cc2C(=O)O)NC1=O]

Imazapyr (2-(4-Methyl-5-oxo-4-propan-2-yl-1H-imidazol-2-yl)pyridine-3-carboxylic acid) is a herbicide for non-crop land applications which is particularly effective on hard-to-control perennial grasses.

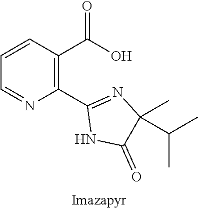 [SMILES: CC(C)C1(C)N=C(c2ncccc2C(=O)O)NC1=O]

Benazolin ethyl (ethyl 2-(4-chloro-2-oxo-2,3-dihydro-1,3-benzothiazol-3-yl)acetate) is a post emergence herbicide that does not have EU approval for use. It has a low aqueous solubility, is volatile with a low tendency to leach to groundwater. It is not persistent in soil systems but may be persistent in water. Benazolin ethyl has a low mammalian toxicity and is not expected to bioaccumulation. It has a low toxicity to birds and is moderately toxicity to most aquatic organisms and earthworms.

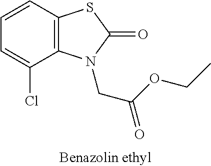 [SMILES: CCOC(=O)Cn1c(=O)sc2cccc(Cl)c21]

Diflufenzopyr (2-[1-[2-[[(3,5-difluorophenyl)amino]carbonyl]hydrazinylidene]ethyl]-3-pyridinecarboxylic acid) is used as a herbicide, post-emergence, to control annual broad-leaved and perennial weeds in a variety of crops.

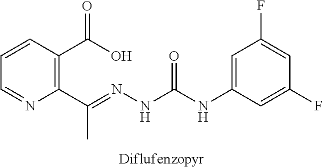 [SMILES: C/C(=N\NC(=O)Nc1cc(F)cc(F)c1)c1ncccc1C(=O)O]

Naptalam (2-[(1-naphthalenylamino)carbonyl]benzoic acid) is a herbicide without EU approval for use. It is usually used as the sodium salt as this is more soluble. Whilst naptalam is not particularly toxic to humans its major metabolite 1-naphthylamine is a carcinogen. Its environmental fate is pH sensitive. It may pose a threat to groundwaters. Virtually non toxic to mammals, birds, honeybees and most aquatic species. No data for earthworms has been identified.

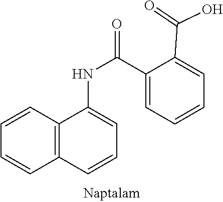 [SMILES: O=C(O)c1ccccc1C(=O)Nc1cccc2ccccc12]

Acibenzolar-S-Methyl (S-Methyl 1,2,3-benzothiadiazole-7-carbothioate) is a fungicide.

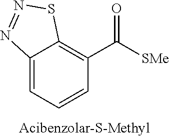 [SMILES: CSC(=O)c1cccc2nnsc12]

Bifenox (2,4-dichlorophenyl 3′-carbomethoxy-4′-nitrophenyl ether) is a herbicide approved for use in the EU. It has a low aqueous solubility, is volatile and would not be expected to leach to groundwater. It would also not be expected to persistent in soil or water systems. It has a low mammalian toxicity and no serious health concerns have been identified. Bifenox is moderately toxic to fish, aquatic invertebrates and earthworms but is highly toxic to algae.

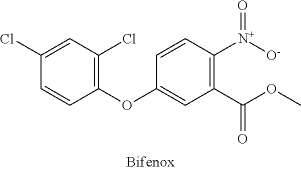 [SMILES: COC(=O)c1cc(Oc2ccc(Cl)cc2Cl)ccc1[N+](=O)[O-]]

Cinidon-ethyl (ethyl (2Z)-2-chloro-3-[2-chloro-5-(1,3,4,5,6,7-hexahydro-1,3-dioxo-2H-isoindol-2-yl)phenyl]prop-2-enoate) is a post-emergence herbicide that has EU approval for use. It has a low aqueous solubility, is relatively volatile with a low tendency to leach to groundwater. It is slightly mobile. It is not persistent in soil or water systems. Cinidon-ethyl has a low mammalian toxicity and is not expected to bioaccumulate. It is moderately toxic to earthworms but more toxic to aquatic organisms. It is relatively non-toxic to honeybees.

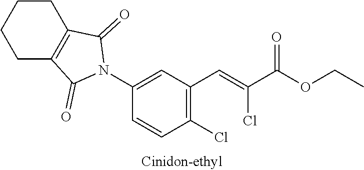 [SMILES: CCOC(=O)/C(Cl)=C/c1cc(N2C(=O)C3=C(CCCC3)C2=O)ccc1Cl]

In a first aspect, the invention comprises a compound of Formula (A);

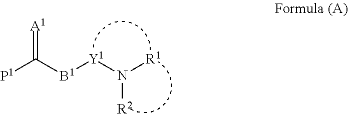

wherein;
dotted lines indicate that the indicated atoms may form part of a cyclic structure;
P¹ is selected from the group consisting of;

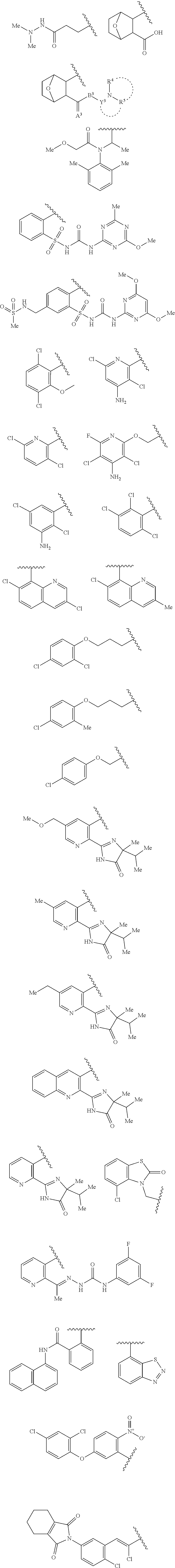

A¹ is O or S;
B¹ is O, S or N;
Y¹ is C₂-C₉ alkyl, straight, branched or cyclic, unsubstituted or substituted by halogen, alkoxy, cyano, hydroxyl, alkylamine or aryl, may be interrupted by O, S or >NR³;
R¹ is H; aryl; C₁-C₆ alkyl, straight, branched or cyclic, unsubstituted or substituted by halogen, alkoxy, cyano, hydroxyl, alkylamine or aryl, and may be interrupted by one or more of O, S or >NR₃, provided that any multiple interruptions are not consecutive;
R³ is C₁-C₆ alkyl;
R² is H; aryl; C₁-C₆ alkyl, straight, branched or cyclic, unsubstituted or substituted by halogen, alkoxy, cyano, hydroxyl, alkylamine or aryl, and may be interrupted by one or more of O, S or >NR³ provided that any multiple interruptions are not consecutive; or R² is;

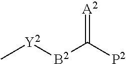

and wherein;

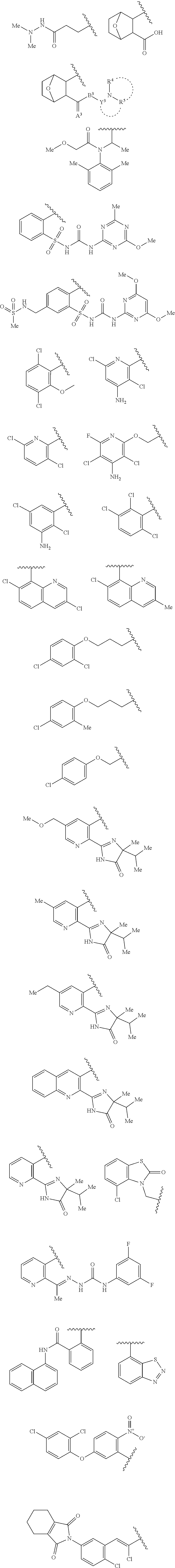

A² is O or S;
B² is O, S or N;
Y² is C₂-C₉ alkyl, straight, branched or cyclic, unsubstituted or substituted by halogen, alkoxy, cyano, hydroxyl, alkylamine or aryl, may be interrupted by one or more of O, S or >NR₃ provided that any multiple interruptions are not consecutive;
   and wherein;
A³ is O or S;
B³ is O, S or N;
Y³ is C₂-C₉ alkyl, straight, branched or cyclic, unsubstituted or substituted by halogen, alkoxy, cyano, hydroxyl, alkylamine or aryl, may be interrupted by O, S or >NR⁶;
R⁴ is H; aryl; C₁-C₆ alkyl, straight, branched or cyclic, unsubstituted or substituted by halogen, alkoxy, cyano, hydroxyl, alkylamine or aryl, and may be interrupted by one or more of O, S or >NR₃, provided that any multiple interruptions are not consecutive;
R⁵ is H; aryl; C₁-C₆ alkyl, straight, branched or cyclic, unsubstituted or substituted by halogen, alkoxy, cyano, hydroxyl, alkylamine or aryl, and may be interrupted by one or more of O, S or >NR⁶ provided that any multiple interruptions are not consecutive;
R⁶ is C₁-C₆ alkyl;
   and salts or solvates thereof; with the proviso that the compound is not 2-(dimethylamino)ethyl 3,6-dichloro-2-methoxybenzoate or 2-(4-chlorophenoxy)acetic acid 2-(dimethylamino)ethyl ester.

The phrase “may be interrupted by” means that the heteroatom or heteroatoms is/are included in addition to the carbon atoms in the alkyl chain. For example, the compounds of Formula (A) include compounds where Y² is a chain comprising nine carbon atoms and one or more oxygen atoms.

In a preferred form of the invention, the compound is not 2-(dimethylamino)ethyl 2-[(4-amino-3,5-dichloro-6-fluoro-2-pyridinyl)oxy]acetate.

In preferred forms of the invention, R² is H; aryl; C₁-C₆ alkyl, straight, branched or cyclic, unsubstituted or substituted by halogen, alkoxy, cyano, hydroxyl, alkylamine or aryl, and may be interrupted by one or more of O, S or >NR³ provided that any multiple interruptions are not consecutive.

In preferred forms of the invention, where R² is

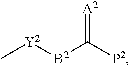

P¹=P².

In preferred forms of the invention, where R² is

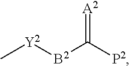

Y²=Y¹, B²=B¹, and/or A²=A¹.

In preferred forms of the invention, where R² is

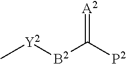

and where P² is

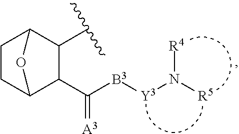

Y³=Y²=Y¹, B³=B²=B¹, A³=A²=A¹, R⁴=R¹, R⁵=R².

In a one form of the invention, P¹ and, where R² is

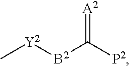

P² are, independently, selected from the group consisting of:

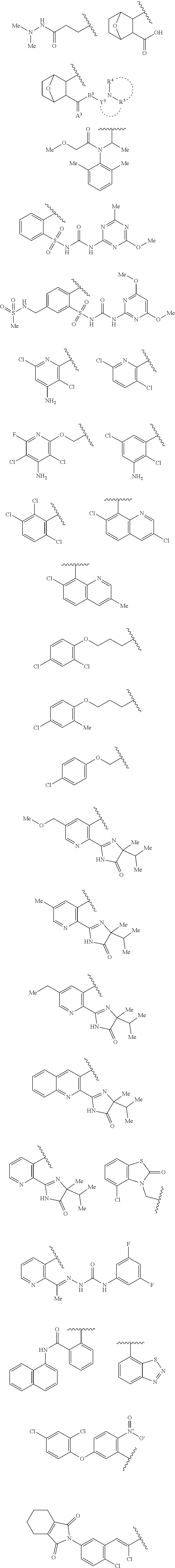

In one form of the invention, P¹ and, where R² is

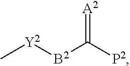

P² are, independently, selected from the group consisting of:

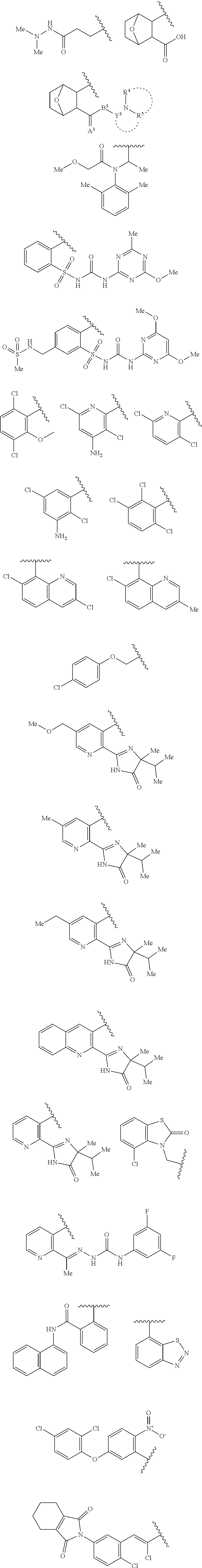

In one form of the invention, P¹ and, where R² is

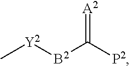

P² are, independently, selected from the group consisting of:

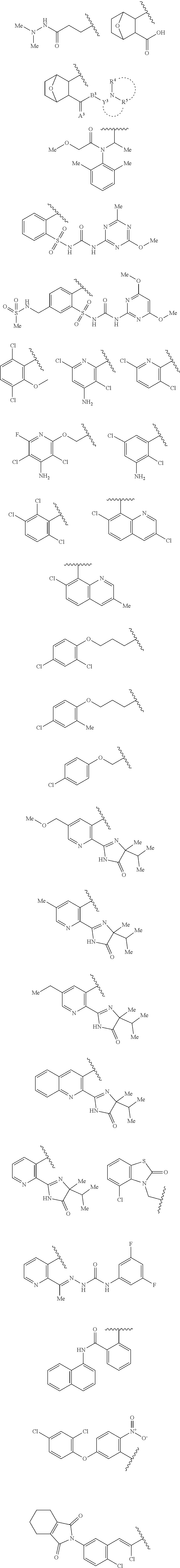

In one form of the invention, P¹ and, where R² is

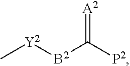

P² are independently is selected from the group consisting of:

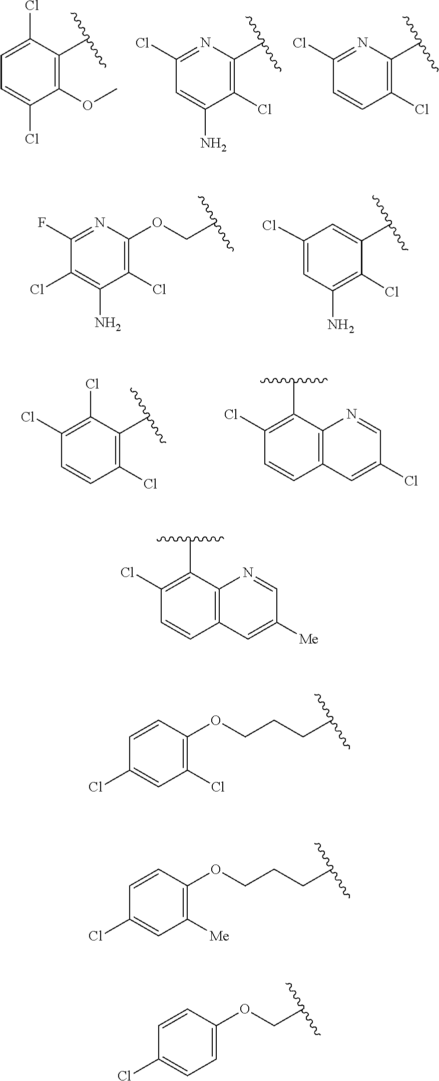

In one form of the invention, P¹ and, where R² is

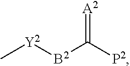

P² are, independently, selected from the group consisting of:

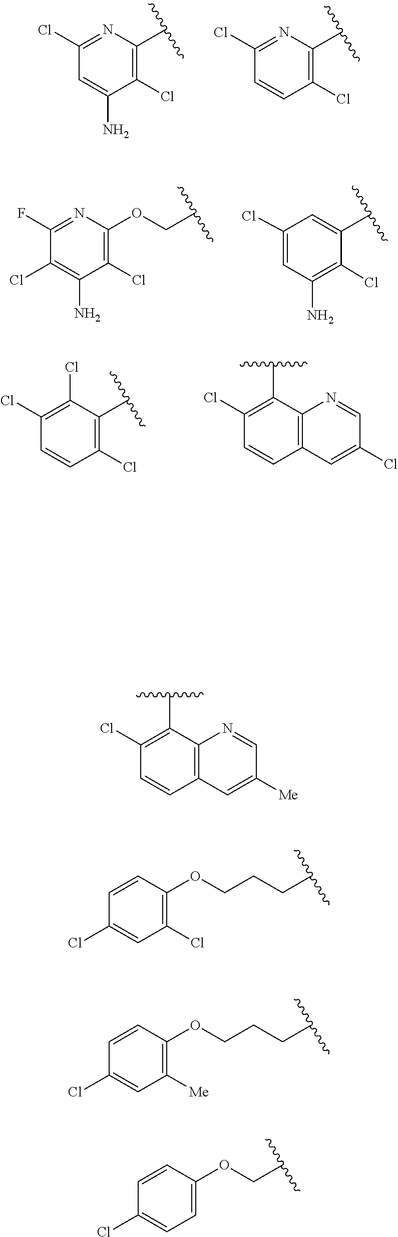

In one form of the invention, P¹ and, where R² is

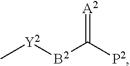

P² are, independently, selected from the group consisting of:

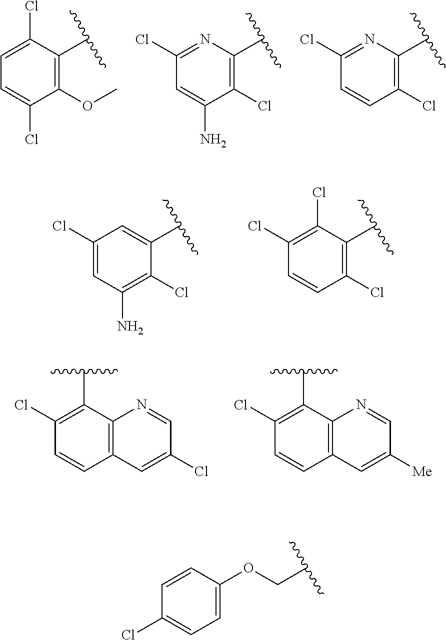

In a preferred form of the invention;
A¹ is O;
B¹ is O or S;
Y¹ is C₂-C₆ alkyl, straight, branched or cyclic, may be interrupted by O; and
R¹ and R² are, independently, C₁-C₆ alkyl, straight, branched or cyclic, may be interrupted by O.

In a preferred form of the invention, the compound of Formula (A) is selected from the list:
2-(diethylamino)ethyl 3,6-dichloro-2-methoxybenzoate;
2-(dipropan-2-ylamino)ethyl 3,6-dichloro-2-methoxybenzoate;
2-(dibutylamino)ethyl 3,6-dichloro-2-methoxybenzoate;
2-(pyrrolidin-1-yl)ethyl 3,6-dichloro-2-methoxybenzoate;
2-(piperidin-1-yl)ethyl 3,6-dichloro-2-methoxybenzoate;
2-(morpholin-4-yl)ethyl 3,6-dichloro-2-methoxybenzoate;
2-(4-methylpiperazin-1-yl)ethyl 3,6-dichloro-2-methoxybenzoate;
1-methylpiperidin-4-yl 3,6-dichloro-2-methoxybenzoate;
(1-methylpyrrolidin-2-yl)methyl 3,6-dichloro-2-methoxybenzoate;
2-(pyridin-2-yl)ethyl 3,6-dichloro-2-methoxybenzoate;
3-(dimethylamino)propyl 3,6-dichloro-2-methoxybenzoate;
2-((2-hydroxyethyl)(methyl)amino)ethyl 3,6-dichloro-2-methoxybenzoate;
(methylazanediyl)bis(ethane-2,1-diyl) bis(3,6-dichloro-2-methoxybenzoate);
2-(ethyl(phenyl)amino)ethyl 3,6-dichloro-2-methoxybenzoate;
2-(dibenzylamino)ethyl 3,6-dichloro-2-methoxybenzoate;
1,3-bis(dimethylamino)propan-2-yl 3,6-dichloro-2-methoxybenzoate;
S-(2-(dimethylamino)ethyl) 3,6-dichloro-2-methoxybenzene-1-carbothioate;
S-(2-(diethylamino)ethyl) 3,6-dichloro-2-methoxybenzene-1-carbothioate;
3,6-dichloro-N-(2-(dimethylamino)ethyl)-2-methoxybenzamide;
2-(2-(dimethylamino)ethoxy)ethyl 3,6-dichloro-2-methoxybenzoate;
4-(dimethylamino)butyl 3,6-dichloro-2-methoxybenzoate;
6-(dimethylamino)hexyl 3,6-dichloro-2-methoxybenzoate;
8-(dimethylamino)octyl 3,6-dichloro-2-methoxybenzoate;
2-aminoethyl 3,6-dichloro-2-methoxybenzoate;
2-(methylamino)ethyl 3,6-dichloro-2-methoxybenzoate;
2-amino-2-methylpropyl 3,6-dichloro-2-methoxybenzoate;
1-aminopropan-2-yl 3,6-dichloro-2-methoxybenzoate;
3-aminopropyl 3,6-dichloro-2-methoxybenzoate;
2-(dimethylamino)ethyl 4-(2,4-dichlorophenoxy)butanoate;
3,6-dichloropyridine-2-carboxylic acid 2-(dimethylamino)ethyl ester;
3,7-dichloroquinoline-8-carboxylic acid 2-(dimethylamino)ethyl ester;
N-(2,6-dimethylphenyl)-N-(methoxyacetyl)-DL-alanine 2-(dimethylamino)ethyl ester;
2-(dimethylamino)ethyl 2-[4,5-dihydro-4-methyl-4-(1-methylethyl)-5-oxo-1H-imidazol-2-yl]-3-pyridinecarboxylate;
2-(dimethylamino)ethyl 2-[1-[2-[[(3-fluorophenyl)amino]carbonyl]hydrazinylidene]ethyl]-3-pyridinecarboxylate.

In a preferred form, the invention comprises a compound of Formula (A1);

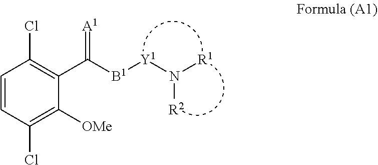

wherein;
dotted lines indicate that the indicated atoms may form part of a cyclic structure;
A¹ is O or S;
B¹ is O, S or N;
Y¹ is C₂-C₉ alkyl, straight, branched or cyclic, unsubstituted or substituted by halogen, alkoxy, cyano, hydroxyl, carbonyl, sulfonyl, sulfinyl, alkylamine or aryl, may be interrupted by O, S or >NR₃;
R¹ is H; aryl; C₁-C₆ alkyl, straight, branched or cyclic, unsubstituted or substituted by halogen, alkoxy, cyano, hydroxyl, carbonyl, sulfonyl, sulfinyl, alkylamine or aryl, and may be interrupted by one or more of O, S or >NR₃, provided that any multiple interruptions are not consecutive;
R³ is C₁-C₆ alkyl;
R² is H; aryl; C₁-C₆ alkyl, straight, branched or cyclic, unsubstituted or substituted by halogen, alkoxy, cyano, hydroxyl, carbonyl, sulfonyl, sulfinyl, alkylamine or aryl, and may be interrupted by one or more of O, S or >NR₃ provided that any multiple interruptions are not consecutive;
or R² is

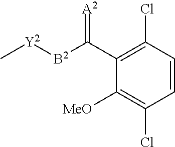

and wherein;
A² is O or S;
B² is O, S or N;
Y² is C₂-C₉ alkyl, straight, branched or cyclic, unsubstituted or substituted by halogen, alkoxy, cyano, hydroxyl, carbonyl, sulfonyl, sulfinyl, alkylamine or aryl, may be interrupted by one or more of O, S or >NR₃ provided that any multiple interruptions are not consecutive;
   and salts or solvates thereof; with the proviso that the compound is not 2-(dimethylamino)ethyl 3,6-dichloro-2-methoxybenzoate.

The phrase “may be interrupted by” means that the heteroatom or heteroatoms is/are included in addition to the carbon atoms in the alkyl chain. For example, the compounds of Formula (A) include compounds where Y² is a chain comprising nine carbon atoms and one or more oxygen atoms.

In a preferred form of the invention;
A¹ is O;
B¹ is O or S;
Y¹ is C₂-C₆ alkyl, straight, branched or cyclic, may be interrupted by O; and
R¹ and R² are, independently, C₁-C₆ alkyl, straight, branched or cyclic, may be interrupted by O, with the proviso that the compound is not 2-(dimethylamino)ethyl 3,6-dichloro-2-methoxybenzoate.

In one form of the invention, the compound of Formula (A1) is a compound selected from the group consisting of:

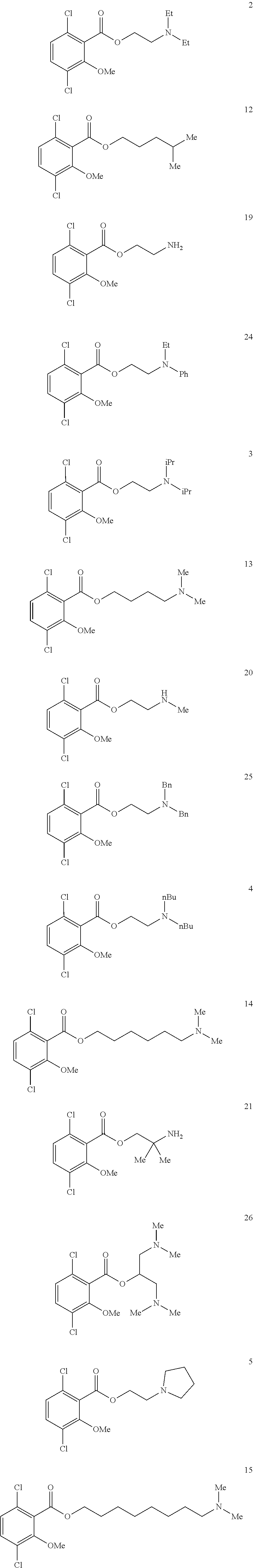

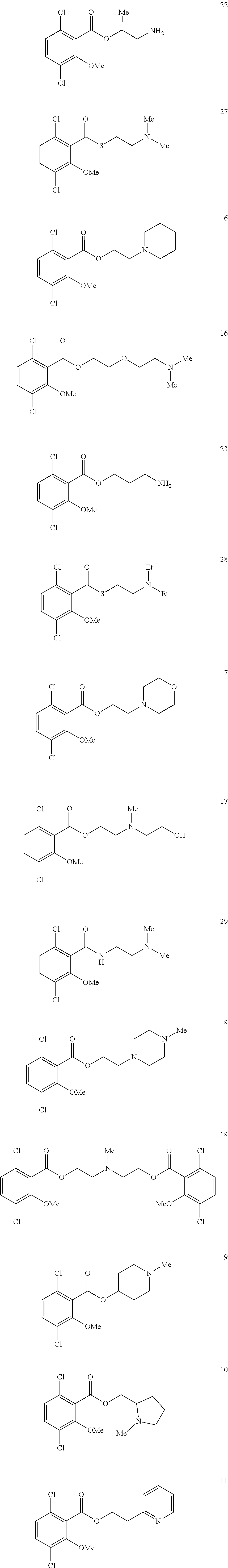

In one form of the invention, the compound of Formula (A1) is a compound selected from the group consisting of:

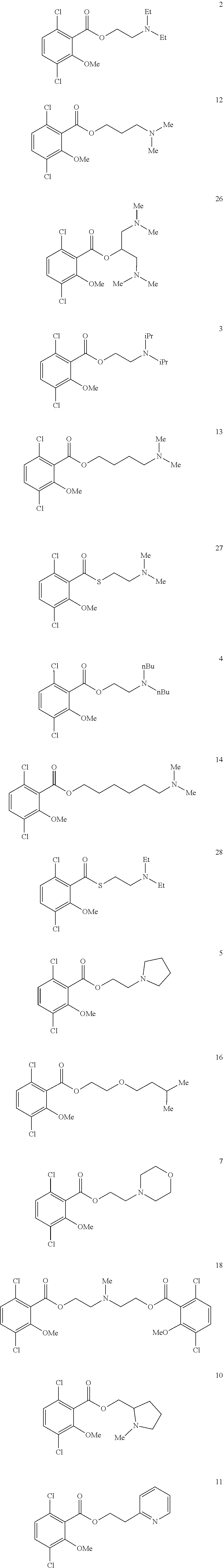

In a preferred form, the invention comprises a salt of the compound of Formula (A1). Preferably still, the salt is an agriculturally acceptable salt.

In one form of the invention, the salt is selected from the group consisting of: chloride, bromide, iodide, bromide, acetate, dihydrogen phosphate, hydrogen sulphate, mesylate, tosylate, citrate, salicylate, fatty acid anions.

In a preferred form of the invention, the salt is selected from the group consisting of: chloride, acetate, dihydrogen phosphate, hydrogen sulphate, citrate.

Further and still preferably, the salt is a hydrochloride salt.

In a preferred form of the invention, the compound is a salt of the compound 2-(dimethylamino)ethyl 3,6-dichloro-2-methoxybenzoate, with the proviso that the compound is not the hydrochloride salt.

Without wishing to be bound by theory, the inventors understand that the compounds of the invention are absorbed by the target plant intact, which would explain the improved volatility properties of certain compounds of the compounds of the invention.

Agricultural Compositions

In a second aspect, the invention comprises an agricultural composition comprising an effective amount of a compound of Formula (B), including salts and solvates thereof, and an agriculturally acceptable adjuvant or carrier;

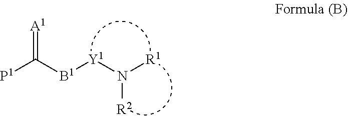

wherein;
dotted lines indicate that the indicated atoms may form part of a cyclic structure;
P¹ is selected from the group consisting of;

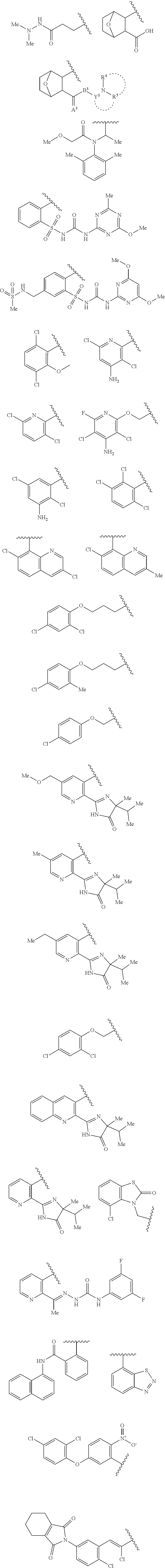

A¹ is O or S;
B¹ is O or S;
Y¹ is C₂-C₉ alkyl, straight, branched or cyclic, unsubstituted or substituted by halogen, alkoxy, cyano, hydroxyl, alkylamine or aryl, may be interrupted by O, S or >NR³;
R¹ is H; aryl; C₁-C₆ alkyl, straight, branched or cyclic, unsubstituted or substituted by halogen, alkoxy, cyano, hydroxyl, alkylamine or aryl, and may be interrupted by one or more of O, S or >NR₃, provided that any multiple interruptions are not consecutive;
R³ is C₁-C₆ alkyl;
R² is H; aryl; C₁-C₆ alkyl, straight, branched or cyclic, unsubstituted or substituted by halogen, alkoxy, cyano, hydroxyl, alkylamine or aryl, and may be interrupted by one or more of O, S or >NR³ provided that any multiple interruptions are not consecutive; or R² is

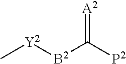

and wherein;
P² is, independently of P¹ selected from the group consisting of;

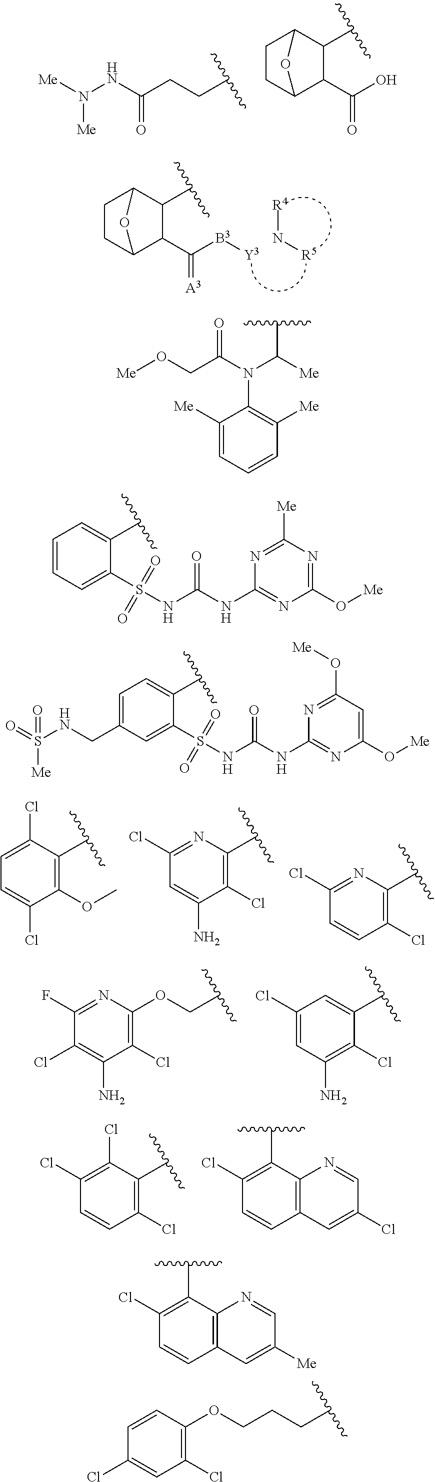

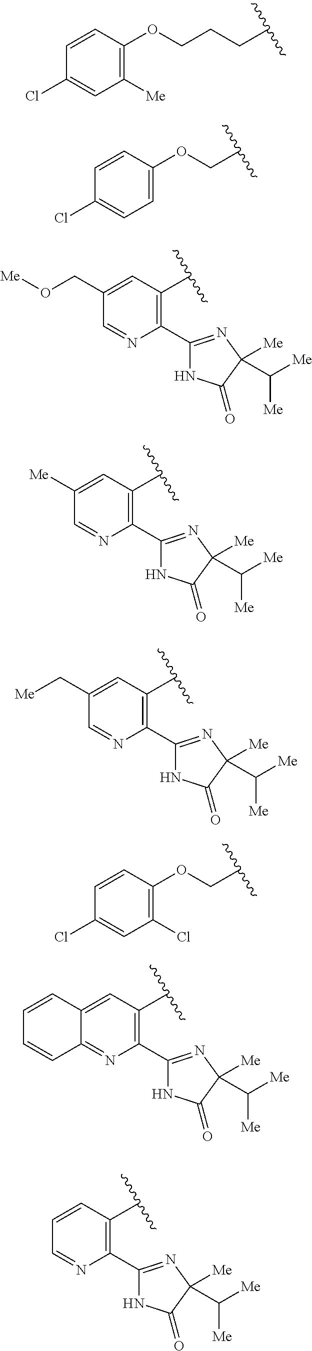

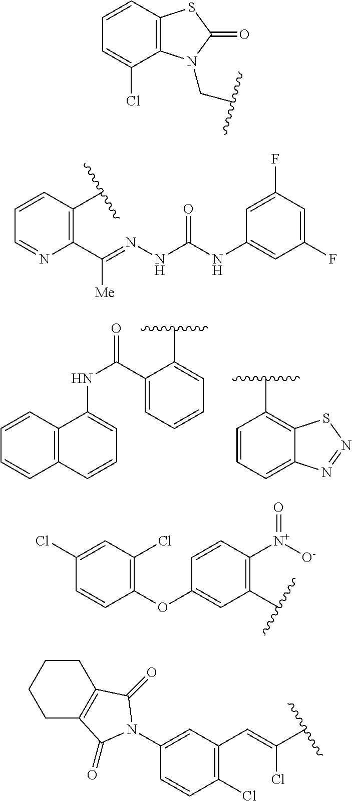

A² is O or S;
B² is O or S;
Y² is C₂-C₉ alkyl, straight, branched or cyclic, unsubstituted or substituted by halogen, alkoxy, cyano, hydroxyl, alkylamine or aryl, may be interrupted by one or more of O, S or >NR₃ provided that any multiple interruptions are not consecutive;
   and wherein;
A³ is O or S;
B³ is O or S;
Y³ is C₂-C₉ alkyl, straight, branched or cyclic, unsubstituted or substituted by halogen, alkoxy, cyano, hydroxyl, alkylamine or aryl, may be interrupted by O, S or >NR⁶;
R⁴ is H; aryl; C₁-C₆ alkyl, straight, branched or cyclic, unsubstituted or substituted by halogen, alkoxy, cyano, hydroxyl, alkylamine or aryl, and may be interrupted by one or more of O, S or >NR₃, provided that any multiple interruptions are not consecutive;
R⁵ is H; aryl; C₁-C₆ alkyl, straight, branched or cyclic, unsubstituted or substituted by halogen, alkoxy, cyano, hydroxyl, alkylamine or aryl, and may be interrupted by one or more of O, S or >NR⁶ provided that any multiple interruptions are not consecutive;
R⁶ is C₁-C₆ alkyl.

The phrase “may be interrupted by” means that the heteroatom or heteroatoms is/are included in addition to the carbon atoms in the alkyl chain. For example, the compounds of Formula (A) include compounds where Y² is a chain comprising nine carbon atoms and one or more oxygen atoms.

In one form of the invention, the compound of Formula (B) is not 2-(dimethylamino)ethyl 2-(2,4-dichlorophenoxy)acetate.

In preferred forms of the invention, R² is H; aryl; C₁-C₆ alkyl, straight, branched or cyclic, unsubstituted or substituted by halogen, alkoxy, cyano, hydroxyl, alkylamine or aryl, and may be interrupted by one or more of O, S or >NR³ provided that any multiple interruptions are not consecutive.

In preferred forms of the invention, where R² is

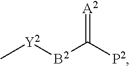

P¹=P².

In preferred forms of the invention, where R² is

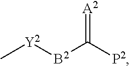

Y²=Y¹, B²=B¹, and/or A²=A¹.

In preferred forms of the invention, where R² is

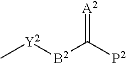

and where P² is

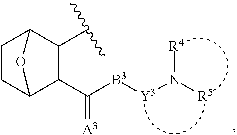

Y³=Y²=Y¹, B³=B²=B¹, A³=A²=A¹, R⁴=R¹, R⁵=R².

In a one form of the invention, P¹ and, where R² is

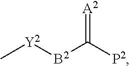

P² are, independently, selected from the group consisting of:

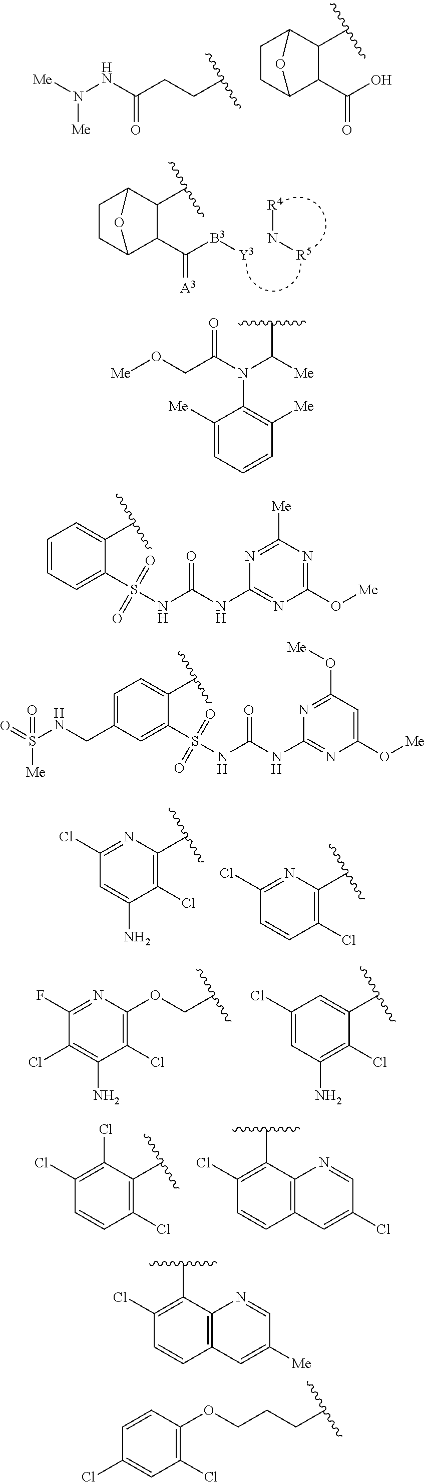

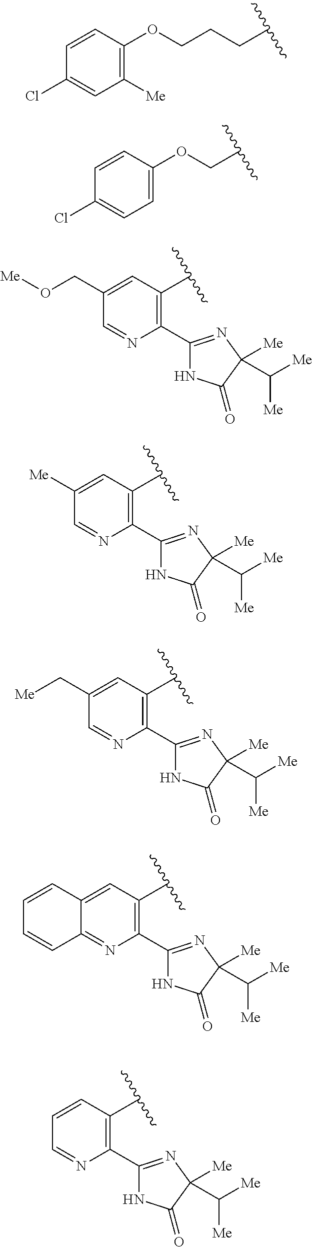

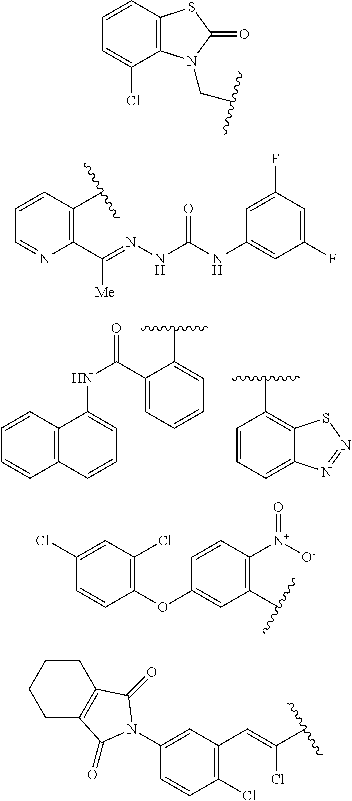

In one form of the invention, P¹ and, where R² is

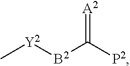

P² are, independently, selected from the group consisting of:

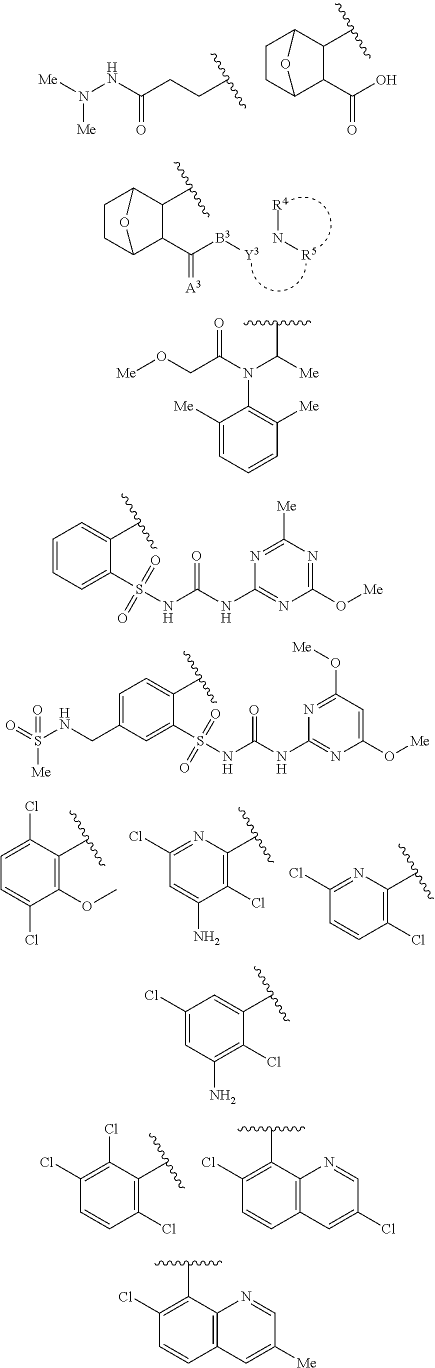

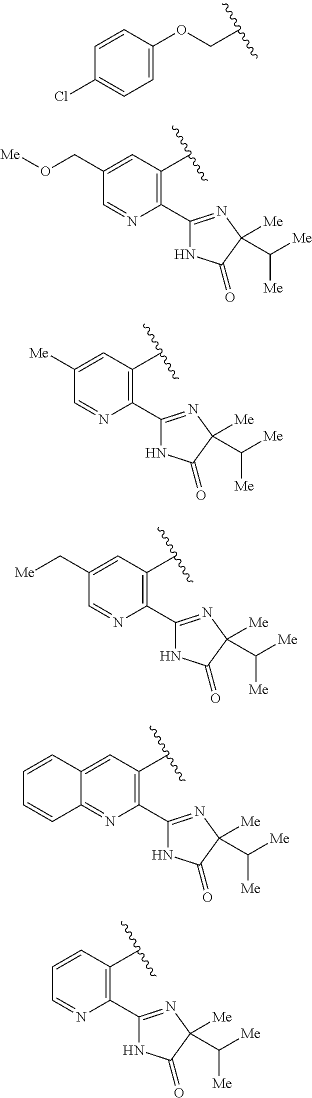 [SMILES: *COc1ccc(Cl)cc1 ; *c1cc(COC)cnc1C1=NC(C)(C(C)C)C(=O)N1 ; *c1cc(C)cnc1C1=NC(C)(C(C)C)C(=O)N1 ; *c1cc(CC)cnc1C1=NC(C)(C(C)C)C(=O)N1 ; *c1cc2ccccc2nc1C1=NC(C)(C(C)C)C(=O)N1 ; *c1cccnc1C1=NC(C)(C(C)C)C(=O)N1]

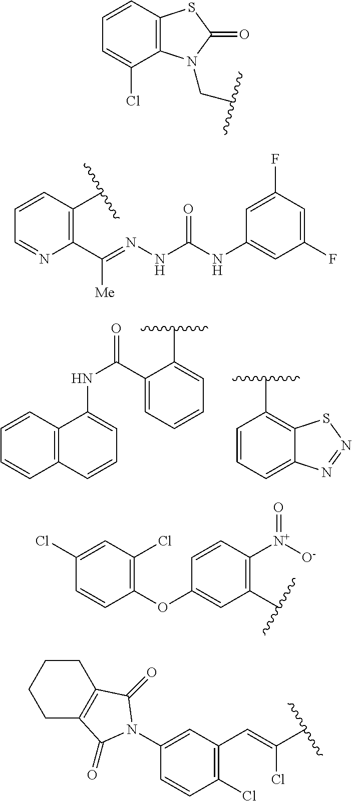

In one form of the invention, P¹ and, where R² is

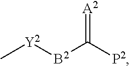

P² are, independently, selected from the group consisting of:

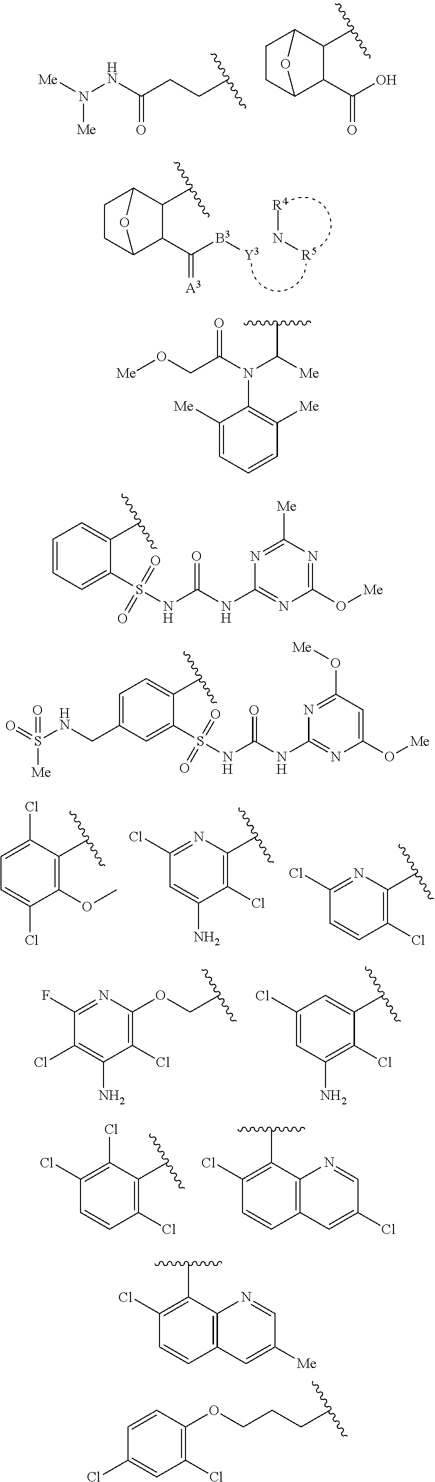

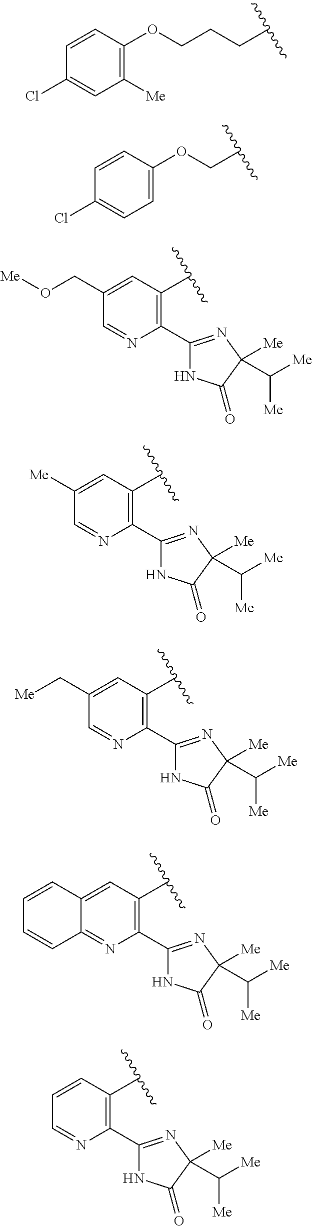

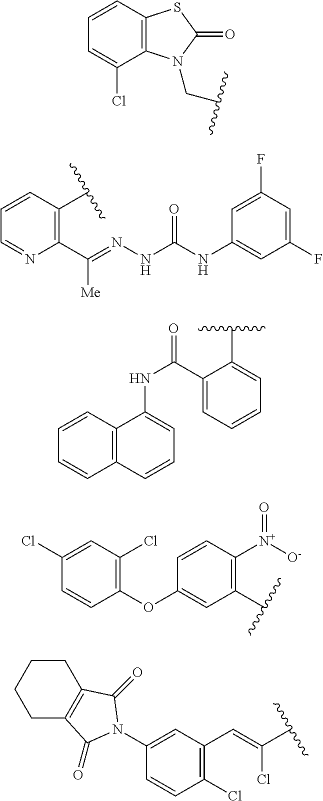 [SMILES: *Cn1c(=O)sc2cccc(Cl)c21 ; *c1cccnc1/C(C)=N/NC(=O)Nc1cc(F)cc(F)c1 ; *c1ccccc1C(=O)Nc1cccc2ccccc12 ; *c1cc(Oc2ccc(Cl)cc2Cl)ccc1[N+](=O)[O-] ; */C(Cl)=C/c1cc(N2C(=O)C3=C(CCCC3)C2=O)ccc1Cl]

In one form of the invention, P¹ and, where R² is

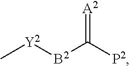

P² are independently is selected from the group consisting of:

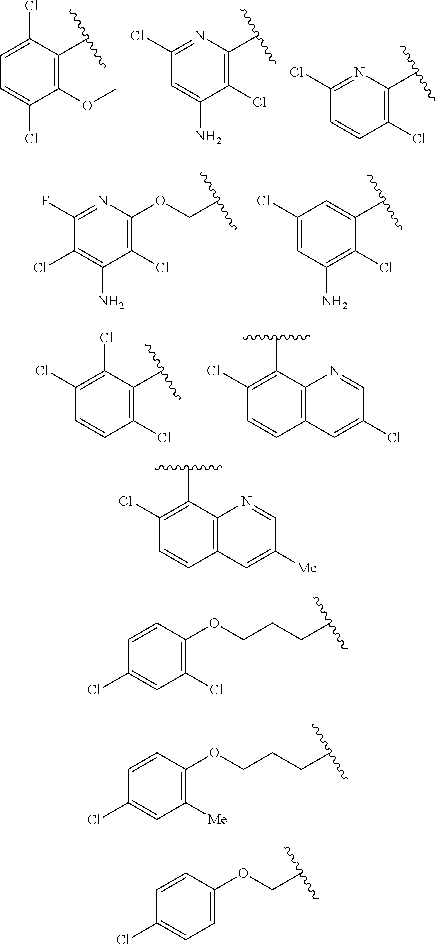

In one form of the invention, P¹ and, where R² is

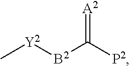

P² are, independently, selected from the group consisting of:

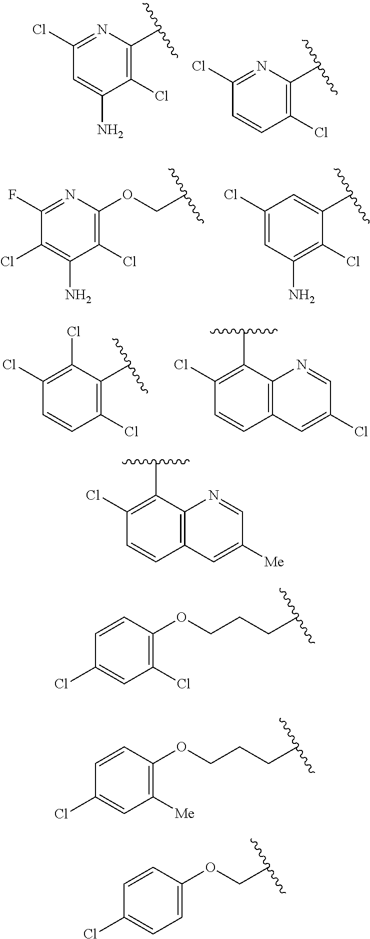

In one form of the invention, P¹ and, where R² is

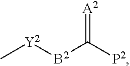

P² are, independently, selected from the group consisting of:

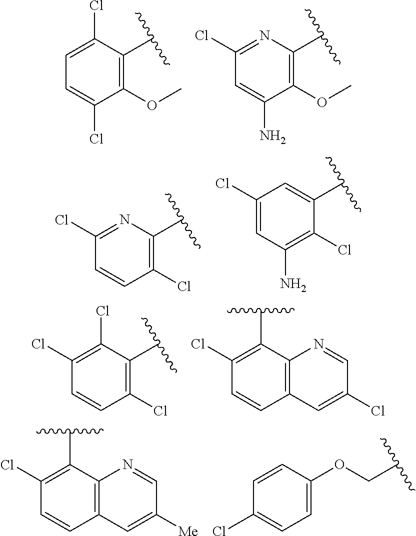

In a preferred form of the invention;
A¹ is O;
B¹ is O or S;
Y¹ is C₂-C₆ alkyl, straight, branched or cyclic, may be interrupted by O; and
R¹ and R² are, independently, C₁-C₆ alkyl, straight, branched or cyclic, may be interrupted by O.

In a preferred form of the invention, the compound of Formula (A) is selected from the list:
2-(diethylamino)ethyl 3,6-dichloro-2-methoxybenzoate;
2-(dipropan-2-ylamino)ethyl 3,6-dichloro-2-methoxybenzoate;
2-(dibutylamino)ethyl 3,6-dichloro-2-methoxybenzoate;
2-(pyrrolidin-1-yl)ethyl 3,6-dichloro-2-methoxybenzoate;
2-(piperidin-1-yl)ethyl 3,6-dichloro-2-methoxybenzoate;
2-(morpholin-4-yl)ethyl 3,6-dichloro-2-methoxybenzoate;
2-(4-methylpiperazin-1-yl)ethyl 3,6-dichloro-2-methoxybenzoate;
1-methylpiperidin-4-yl 3,6-dichloro-2-methoxybenzoate;
(1-methylpyrrolidin-2-yl)methyl 3,6-dichloro-2-methoxybenzoate;
2-(pyridin-2-yl)ethyl 3,6-dichloro-2-methoxybenzoate;
3-(dimethylamino)propyl 3,6-dichloro-2-methoxybenzoate;
2-((2-hydroxyethyl)(methyl)amino)ethyl 3,6-dichloro-2-methoxybenzoate;
(methylazanediyl)bis(ethane-2,1-diyl) bis(3,6-dichloro-2-methoxybenzoate);
2-(ethyl(phenyl)amino)ethyl 3,6-dichloro-2-methoxybenzoate;
2-(dibenzylamino)ethyl 3,6-dichloro-2-methoxybenzoate;
1,3-bis(dimethylamino)propan-2-yl 3,6-dichloro-2-methoxybenzoate;
S-(2-(dimethylamino)ethyl) 3,6-dichloro-2-methoxybenzene-1-carbothioate;
S-(2-(diethylamino)ethyl) 3,6-dichloro-2-methoxybenzene-1-carbothioate;
3,6-dichloro-N-(2-(dimethylamino)ethyl)-2-methoxybenzamide;
2-(2-(dimethylamino)ethoxy)ethyl 3,6-dichloro-2-methoxybenzoate;
4-(dimethylamino)butyl 3,6-dichloro-2-methoxybenzoate;
6-(dimethylamino)hexyl 3,6-dichloro-2-methoxybenzoate;
8-(dimethylamino)octyl 3,6-dichloro-2-methoxybenzoate;
2-aminoethyl 3,6-dichloro-2-methoxybenzoate;
2-(methylamino)ethyl 3,6-dichloro-2-methoxybenzoate;
2-amino-2-methylpropyl 3,6-dichloro-2-methoxybenzoate;
1-aminopropan-2-yl 3,6-dichloro-2-methoxybenzoate;
3-aminopropyl 3,6-dichloro-2-methoxybenzoate;
2-(dimethylamino)ethyl 4-(2,4-dichlorophenoxy)butanoate;
3,6-dichloropyridine-2-carboxylic acid 2-(dimethylamino)ethyl ester;
3,7-dichloroquinoline-8-carboxylic acid 2-(dimethylamino)ethyl ester;
N-(2,6-dimethylphenyl)-N-(methoxyacetyl)-DL-alanine 2-(dimethylamino)ethyl ester;
2-(dimethylamino)ethyl 2-[4,5-dihydro-4-methyl-4-(1-methylethyl)-5-oxo-1H-imidazol-2-yl]-3-pyridinecarboxylate;
2-(dimethylamino)ethyl 2-[1-[2-[[(3-fluorophenyl)amino]carbonyl]hydrazinylidene]ethyl]-3-pyridinecarboxylate.

In a preferred form of the second aspect, the invention comprises a herbicidal composition comprising a herbicidally-effective amount of a compound of Formula (B1), or salts and solvates thereof, and an agriculturally acceptable adjuvant or carrier:

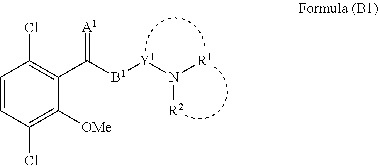

wherein;
dotted lines indicate that the indicated atoms may form part of a cyclic structure;
A¹ is O or S;
B¹ is O or S;
Y¹ is C₂-C₉ alkyl, straight, branched or cyclic, unsubstituted or substituted by halogen, alkoxy, cyano, hydroxyl, carbonyl, sulfonyl, sulfinyl, alkylamine or aryl, may be interrupted by O, S or >NR₃;
R¹ is H; aryl; C₁-C₆ alkyl, straight, branched or cyclic, unsubstituted or substituted by halogen, alkoxy, cyano, hydroxyl, carbonyl, sulfonyl, sulfinyl, alkylamine or aryl, and may be interrupted by one or more of O, S or >NR₃, provided that any multiple interruptions are not consecutive;
R³ is C₁-C₆ alkyl;
R² is H; aryl; C₁-C₆ alkyl, straight, branched or cyclic, unsubstituted or substituted by halogen, alkoxy, cyano, hydroxyl, carbonyl, sulfonyl, sulfinyl, alkylamine or aryl, and may be interrupted by one or more of O, S or >NR₃ provided that any multiple interruptions are not consecutive;
or R² is

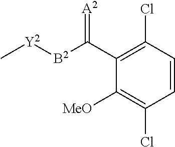

and wherein;
A² is O or S;
B² is O or S;
Y² is C₂-C₉ alkyl, straight, branched or cyclic, unsubstituted or substituted by halogen, alkoxy, cyano, hydroxyl, carbonyl, sulfonyl, sulfinyl, alkylamine or aryl, may be interrupted by one or more of O, S or >NR₃ provided that any multiple interruptions are not consecutive.

The phrase “may be interrupted by” means that the heteroatom or heteroatoms is/are included in addition to the carbon atoms in the alkyl chain. For example, the compounds of Formula (B) include compounds where Y² is a chain comprising nine carbon atoms and one or more oxygen atoms.

In a preferred form of the invention;
A¹ is O;
B¹ is O or S;
Y¹ is C₂-C₆ alkyl, straight, branched or cyclic, may be interrupted by O; and
R¹ and R² are, independently, C₁-C₆ alkyl, straight, branched or cyclic, may be interrupted by O.

In one form of the invention, the compound of Formula (1) is a compound selected from the group consisting of:

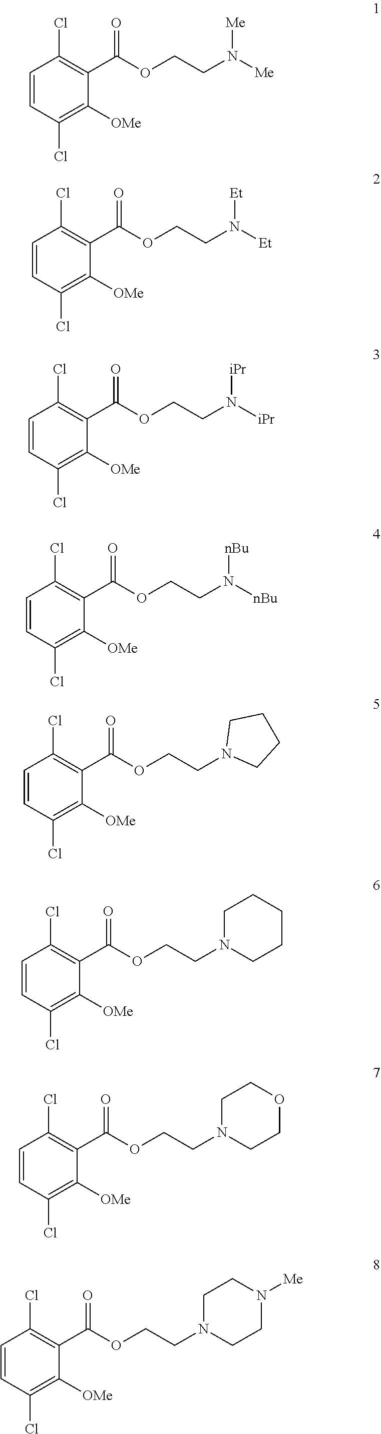

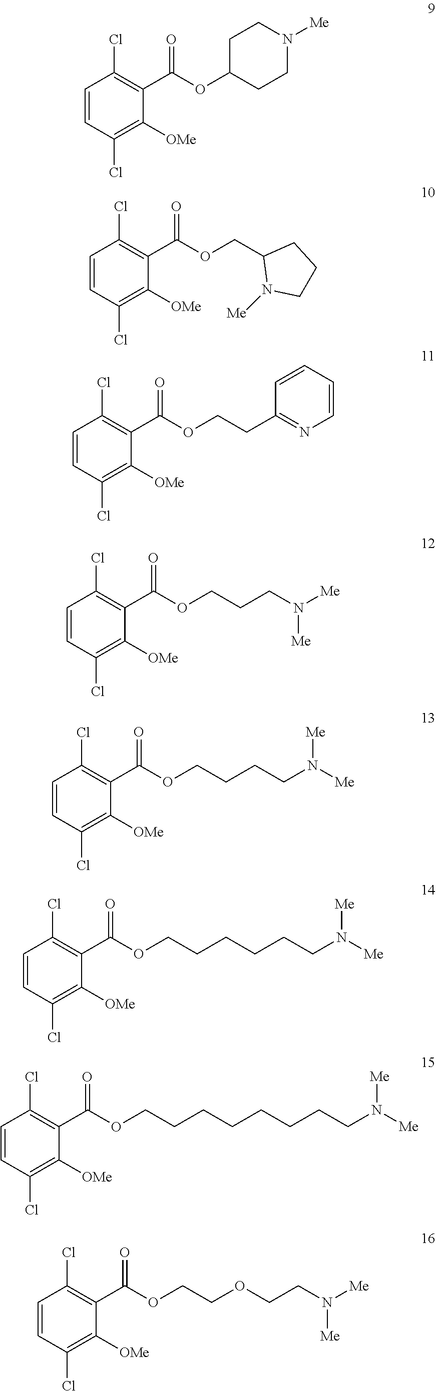

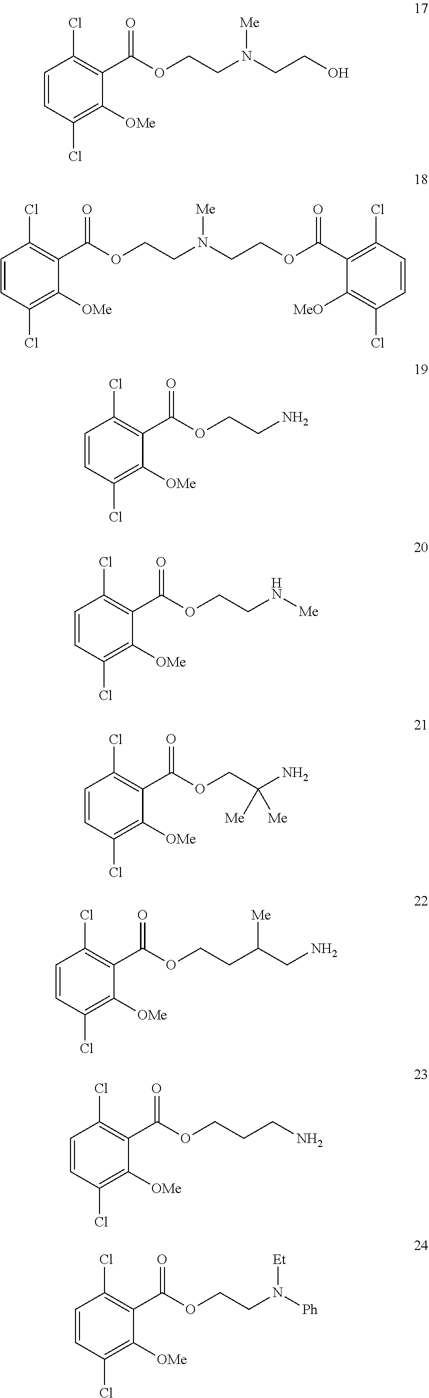

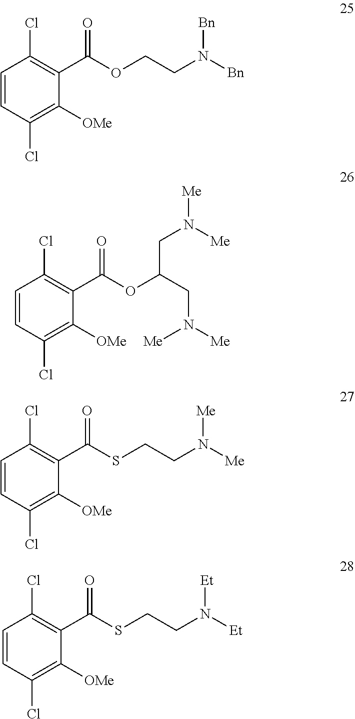

In one form of the invention, the compound of Formula (1) is a compound selected from the group consisting of:

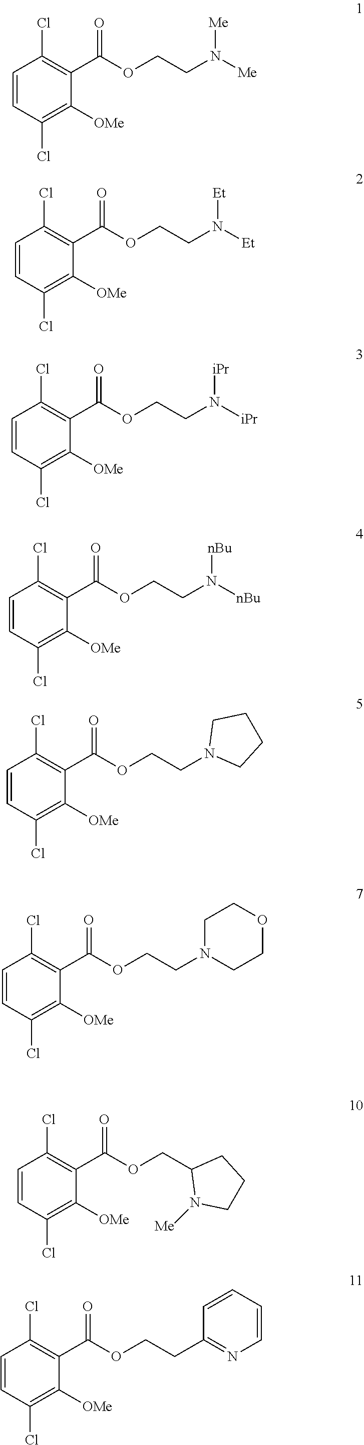

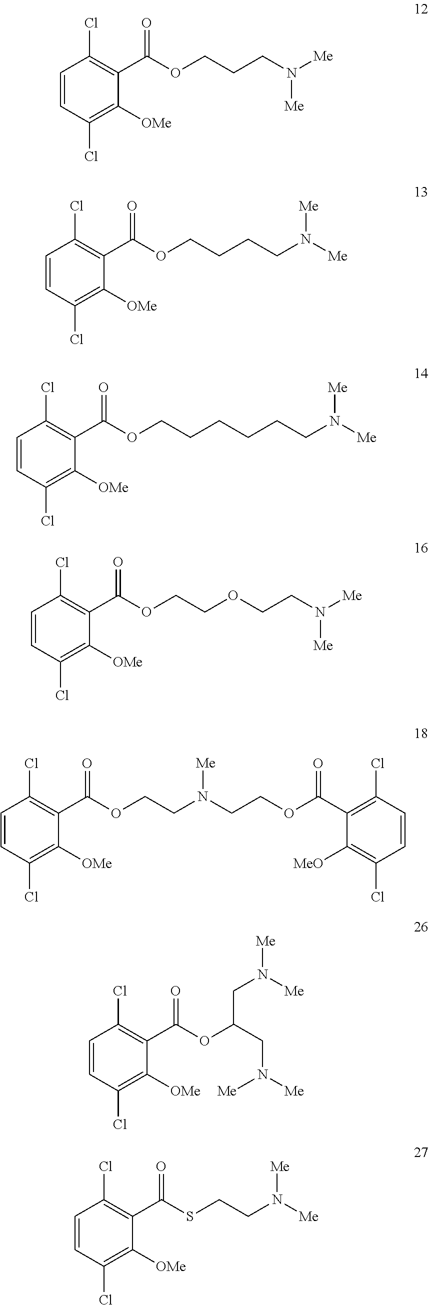

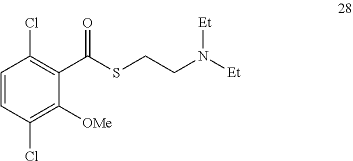 [SMILES: CCN(CC)CCSC(=O)c1c(Cl)ccc(Cl)c1OC]

In a preferred form of the invention, herbicidal composition comprises a salt of a compound of Formula (1). Preferably still, the salt is an agriculturally acceptable salt.

In one form of the invention, the salt is selected from the group consisting of: chloride, bromide, iodide, bromide, acetate, dihydrogen phosphate, hydrogen sulphate, mesylate, tosylate, citrate, salicylate, fatty acid anions.

In a preferred form of the invention, the salt is selected from the group consisting of: chloride, acetate, dihydrogen phosphate, hydrogen sulphate, citrate.

Further and still preferably, the salt is a hydrochloride salt.

In a preferred form of the invention, the herbicidal composition comprises the a salt of the compound 2-(dimethylamino)ethyl 3,6-dichloro-2-methoxybenzoate.

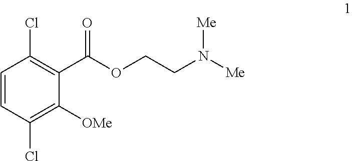 [SMILES: COc1c(Cl)ccc(Cl)c1C(=O)OCCN(C)C]

In a highly preferred form of the invention, the herbicidal composition comprises the hydrochloride salt of 2-(dimethylamino)ethyl 3,6-dichloro-2-methoxybenzoate.

The compounds of the invention be combined with a selection of adjuvants to enhance one or more of the compounds' properties.

Broadly defined, “an adjuvant is an ingredient that aids or modifies the action of the principal active ingredient.” The use of adjuvants with herbicides generally falls into two categories: (1) formulation adjuvants are present in the container when purchased by the dealer or grower; and (2) spray adjuvants are added along with the formulated product to a carrier such as water. The liquid that is sprayed over the top of a crop, weeds, or insect pest often will contain both formulation and spray adjuvants.

Formulation adjuvants may be added to the active ingredient for several reasons, including better mixing and handling, increased effectiveness and safety, better distribution, and drift reduction. These traits are accomplished by altering the solubility, volatility, specific gravity, corrosiveness, shelf-life, compatibility, or spreading and penetration characteristics. With the large number of formulation options available (solutions, emulsions, wettable powders, flowables, granules, and encapsulated materials), adjuvants can be advantageous in assuring consistent performance.

Spray adjuvants may be added to the tank to improve herbicide performance. Literally hundreds of chemical additives are now available that fall into this category. Spray adjuvants can be grouped into two broad categories: (1) activator adjuvants, including surfactants, wetting agents, stickers-spreaders, and penetrants; and (2) special purpose or utility modifiers, such as emulsifiers, dispersants, stabilizing agents, coupling agents, co-solvents, compatibility agents, buffering agents, antifoam agents, drift control agents, and nutritionals.

Thus, agricultural composition, and in specific embodiments the herbicidal composition, may further comprise conventional additives such as drift reduction agents, safeners, thickening agents, flow enhancers, foam-moderating agents, UV protectants, preservatives, antimicrobials solvents, solid carriers, dispersants or emulsifiers (such as further solubilizers, protective colloids, surfactants and adhesion agents), organic and inorganic thickeners, bactericides, anti-freezing agents, anti-foaming agents, colorants and tackifiers or binders (e.g. for seed treatment formulations) that are necessary or desirable to improve the performance, crop safety, or handling of the composition.

Suitable solvents are water, organic solvents such as mineral oil fractions of medium to high boiling point, such as kerosene or diesel oil, furthermore coal tar oils and oils of vegetable or animal origin, aliphatic, cyclic and aromatic hydrocarbons, e. g. toluene, xylene, paraffin, tetrahydronaphthalene, alkylated naphthalenes or their derivatives, alcohols such as methanol, ethanol, propanol, butanol and cyclohexanol, glycols (such as ethylene glycol or 1,2-propylene glycol), ketones such as cyclohexanone and gamma-butyrolactone, fatty acid dimethylamides, fatty acids and fatty acid esters and strongly polar solvents, e.g. amines such as N-methylpyrrolidone.

The preferred solvent is water.

Solid carriers are mineral earths such as silicates, silica gels, talc, kaolins, limestone, lime, chalk, bole, loess, clays, dolomite, diatomaceous earth, calcium sulfate, magnesium sulfate, iron sulfate, magnesium oxide, ground synthetic materials, fertilizers, such as, e.g., ammonium sulfate, ammonium phosphate, ammonium nitrate, ureas, and products of vegetable origin, such as cereal meal, tree bark meal, wood meal and nutshell meal, cellulose powders and other solid carriers.

Suitable surfactants (adjuvants, wetters, tackifiers, dispersants or emulsifiers) are alkali metal, alkaline earth metal and ammonium salts of aromatic sulfonic acids, such as ligninsoulfonic acid (Borresperse® types, Borregard, Norway) phenolsulfonic acid, naphthalenesulfonic acid (Morwet® types, Akzo Nobel, U.S.A.), dibutylnaphthalene-sulfonic acid (Nekal® types, BASF, Germany), and fatty acids, alkylsulfonates, alkylarylsulfonates, alkyl sulfates, laurylether sulfates, fatty alcohol sulfates, and sulfated hexa-, hepta- and octadecanolates, sulfated fatty alcohol glycol ethers, furthermore condensates of naphthalene or of naphthalenesulfonic acid with phenol and formaldehyde, polyoxy-ethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignin-sulfite waste liquors and proteins, denatured proteins, polysaccharides (e. g. methylcellulose), hydrophobically modified starches, polyvinyl alcohols (Mowiol® types, Clariant, Switzerland), polycarboxylates (Sokolan® types, BASF, Germany), polyalkoxylates, polyvinylamines (Lupasol® types, BASF, Germany), polyvinylpyrrolidone and the copolymers thereof. Further suitable surfactants (especially for agrochemical compositions comprising glyphosate) are alkoxylated C4-22-alkylamines, such as ethoxylated tallow amine (POEA) and the surfactants disclosed in EP1389040 (e.g., those in Examples 1 to 14).

Surfactants may be selected from the group consisting of alkoxylated tertiary etheramines, alkoxylated quaternary etheramines, alkoxylated etheramine oxides, alkoxylated tertiary amines, alkoxylated quaternary amines, alkoxylated polyamines, sulfates, sulfonates, phosphate esters, alkyl polysaccharides, alkoxylated alcohols, and combinations thereof.

The weight ratio of the compound equivalent to surfactant can be readily determined by those skilled in the art (e.g., from 1:1 to 20:1, from 2:1 to 10:1 or from 3:1 to 8:1).

Examples for thickeners (i.e., compounds that impart a modified flowability to compositions, i.e., high viscosity under static conditions and low viscosity during agitation) are polysaccharides and organic and anorganic clays such as Xanthan gum (Kelzan®, CP Kelco, U.S.A.), Rhodopol® 23 (Rhodia, France), Veegum® (R.T. Vanderbilt, U.S.A.) or Attaclay® (Engelhard Corp., NJ, USA).

Bactericides may be added for preservation and stabilization of the composition.

Examples for suitable bactericides are those based on dichlorophene and benzylalcohol hemi formal (Proxel® from ICI or Acticide® RS from Thor Chemie and Kathon® MK from Rohm & Haas) and isothiazolinone derivatives such as alkylisothiazolinones and benzisothiazolinones (Acticide® MBS from Thor Chemie).

Examples for suitable anti-freezing agents are ethylene glycol, propylene glycol, urea and glycerin.

Examples for anti-foaming agents are silicone emulsions (such as Silikon® SRE, Wacker, Germany or Rhodorsil®, Rhodia, France), long chain alcohols, fatty acids, salts of fatty acids, fluoroorganic compounds and mixtures thereof.

Examples for tackifiers or binders are polyvinylpyrrolidons, polyvinylacetates, polyvinyl alcohols and cellulose ethers (Tylose®, Shin-Etsu, Japan).

Examples of colorants are both sparingly water-soluble pigments and water-soluble dyes. Examples which may be mentioned are the dyes known under the names Rhodamin B, C.I. Pigment Red 112 and C.I. Solvent Red 1, and also pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

The compounds of the invention may be used in the preparation of concentrates, tank-mixes, or ready-to-use (RTU) formulations. Tank-mix and RTU formulations comprising one or more of the compounds of the invention typically comprise from 0.1 g a.s./L to 50 g a.s./L total herbicide loading, while concentrate formulations typically comprise from 50 to 1000 g a.s./L, from 300 to 1000 g a.s./L, from 350 to 1000 g a.s./L, from 400 to 1000 g a.s./L, from 450 to 1000 g a.s./L, or even from 500 to 1000 g a.s./L total herbicide loading.

In one form of the invention, the herbicidal composition comprises a compound of Formula (B), a salt or solvate thereof, and a polysaccharide.

In one form of the invention, the polysaccharide is selected from the group: galactomannans, chitosan, pectin, alginate, hyaluronic acid, agar, xanthan, dextrin, starch, cellulose, amylose, amylopectin, alteman, gellan, levan, mutan, dextran, pullulan, fructan, gum arabic, carrageenan, glycogen, glycosaminoglycans, murein, xyloglucans, bacterial capsular polysaccharides and combinations thereof.

In one form of the invention, the polysaccharide is selected from the group: galactomannans such as guars, including guar derivatives, xanthans, polyfructoses such as levan, starches, including starch derivatives, such as amylopectin, xyloglucans such as tamarind gum and tamarind gum derivatives such as hydroxypropyl tamarind gum, and cellulose, including cellulose derivatives, such as methylcellulose, ethylcellulose, carboxymethylcellulose, hydroxyethylcellulose, cellulose acetate, cellulose acetate butyrate, and cellulose acetate propionate.

In one form of the invention, the herbicidal composition comprises a compound of the invention and a nitrogen-containing polybasic polymer.

In one form of the invention, the nitrogen-containing polybasic polymer preferably has from 4 to about 100,000 nitrogen atoms per molecule, from about 15 to about 100,000 nitrogen atoms per molecule, from about 25 to about 100,000 nitrogen atoms per molecule, from about 50 to about 100,000 nitrogen atoms per molecule, or even from about 100 to about 100,000 nitrogen atoms per molecule, or mixtures of polybasic polymers having an average number of nitrogen atoms within the above ranges.

For nitrogen containing polybasic polymers or a combination of polymers, an average nitrogen content of from 10% to about 50% by weight, from 13% to about 50%, from 15% to about 50%, from about 20% to about 50%, from about 30% to about 45% by weight, or even about 30% to about 40% by weight is preferred.

In one form of the invention, the herbicidal composition comprises a compound of the invention, a polysaccharide derivative and a nitrogen containing polybasic polymer, as described in International Patent Application WO 2019/158601, the contents of which are incorporated by reference.

The herbicidal compositions of the present invention optionally may further comprise at least one further herbicide.

In one form, the herbicide is a non-auxin herbicide.

The term “non-auxin herbicide” refers to a herbicide having a primary mode of action other than as an auxin herbicide. Representative examples of non-auxin herbicides include acetyl CoA carboxylase (ACCase) inhibitors, acetolactate synthase (ALS) inhibitors, acetohydroxy acid synthase (AHAS) inhibitors, photosystem II inhibitors, photosystem I inhibitors, protoporphyrinogen oxidase (PPO or Protox) inhibitors, carotenoid biosynthesis inhibitors, enolpyruvyl shikimate-3-phosphate (EPSP) synthase inhibitors, glutamine synthetase inhibitors, dihydropteroate synthetase inhibitors, mitosis inhibitors, and nucleic acid inhibitors; salts and esters thereof; racemic mixtures and resolved isomers thereof; and combinations thereof.

In one form of the invention, the non-auxin herbicide is a protoporphyrinogen oxidase (PPO or Protox) inhibitor. For example, the protoporphyrinogen oxidase inhibitor may be the products Luximo™ and/or Tirexor™ by BASF.

The agricultural, and in specific embodiments herbicidal, compositions of the invention may further comprise glyphosate or glufosinate, or an agriculturally acceptable salt thereof such as, for example, the ammonium, diammonium, dimethylammonium, monoethanolamine, isopropylamine, and potassium salt thereof.

The agricultural, and in specific embodiments herbicidal, compositions of the invention may be provided as any of the customary types of agrochemical compositions, e.g., solutions, emulsions, suspensions, dusts, powders, pastes and granules. The composition type depends on the intended purpose; in each case, it should ensure a fine and uniform distribution of the compound according to the invention.

Examples for composition types are suspensions (SC, OD, FS), emulsifiable concentrates (EC), emulsions (EW, EO, ES), pastes, pastilles, wettable powders or dusts (WP, SP, SS, WS, DP, DS) or granules (GR, FG, GG, MG), which can be water-soluble or wettable, as well as gel formulations for the treatment of plant propagation materials such as seeds (GF). Usually the composition types (e. g. SC, OD, FS, EC, WG, SG, WP, SP, SS, WS, GF) are employed diluted. Composition types such as DP, DS, GR, FG, GG and MG are usually used undiluted. The compositions are prepared in a known manner. When the agrochemical composition is an aqueous composition, the salt according to the invention may dissociate into anions and cations.

Among the various composition presentations of the invention are the following:
a. a ready-to-use herbicidal composition that can be applied to unwanted plants without the need for further dilution with a solvent or other preparation;
b. a herbicidal composition concentrate that is diluted with a solvent, e.g. water, and optionally combined with other herbicide and non-herbicide materials, prior to application (including, e.g., dry mixes and premixes);
c. a herbicidal composition application mixture prepared by diluting a herbicidal composition concentrate with a solvent, e.g., water, to form the herbicidal composition application mixture which then can be applied to auxin susceptible plants;
d. a herbicidal composition application mixture prepared by combining two or more separate components with a solvent, e.g., water, (e.g., a tank mix) to form the herbicidal composition application mixture which then can be applied to auxin-susceptible plants; and
e. a herbicidal composition application mixture prepared by introducing separate feed streams to a spraying or application system so that the feed streams are co-mixed to form the herbicidal composition application mixture immediately prior to use.

Powders, materials for spreading and dusts can be prepared by mixing or concomitantly grinding the compounds of the invention and, if appropriate, further active substances, with at least one solid carrier. Granules, e.g., coated granules, impregnated granules and homogeneous granules, can be prepared by binding the active substances to solid carriers.

Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, e.g., ammonium sulfate, ammonium phosphate, ammonium nitrate, ureas, and products of vegetable origin, such as cereal meal, tree bark meal, wood meal and nutshell meal, cellulose powders and other solid carriers.

Methods for Use of the Agricultural Compositions of the Invention.

In a further aspect, the invention comprises a method for use of a compound of Formula (B) or a salt or solvate thereof, wherein the method comprises applying to a plant an effective amount of the compound.

In a preferred form, the invention comprises a method for use of a compound of Formula (1) or a salt or solvate thereof, wherein the method comprises applying to a plant an effective amount of the compound.

In a preferred form, the method comprises applying to the plant an effective amount of an agricultural composition containing a compound of Formula (B), or a salt or solvate thereof.

In a preferred form, the method comprises applying to the plant an effective amount of a herbicidal composition containing a compound of Formula (1), or a salt or solvate thereof.

In a preferred form, the method comprises applying to the plant a herbicidally effective amount of a herbicidal composition containing a compound of Formula (1) or a salt or solvate thereof.

Some herbicidal compositions of the invention are preferably diluted to form an application mixture before being applied to the plant. Application mixtures may be prepared by dissolving the herbicidal composition in water or other suitable solvent or by suitable dilution of a concentrate agricultural composition and applying to the foliage of plants by methods known in the art.

According to anyone of the invention embodiments, the methods of controlling the growth of auxin-susceptible plants comprise the steps of: (a) preparing an aqueous herbicidal application mixture by diluting with water a herbicidal composition concentrate of any of the herbicidal composition concentrates disclosed in this application; and (b) applying a herbicidally effective amount of the application mixture to the auxin-susceptible plants.

The herbicidal compositions of the invention, or application mixtures thereof, can be applied before planting, at planting, pre-emergence, or post-emergence to crop plants, such as in a field of the crop plants to control weeds in a field of the crop plants, depending on the particular herbicide salt and crop plant.

In a further embodiment, the composition according to the invention can be applied by treating seed. The treatment of seed comprises essentially all procedures familiar to the person skilled in the art (seed dressing, seed coating, seed dusting, seed soaking, seed film coating, seed multilayer coating, seed encrusting, seed dripping and seed pelleting) based on the compositions according to the invention. Here, the herbicidal compositions can be applied diluted or undiluted.

The term seed comprises seed of all types, such as, for example, corns, seeds, fruits, tubers, seedlings and similar forms. Here, preferably, the term seed describes corns and seeds.

The present invention further provides a method for treating an agricultural field comprising spraying the field with a herbicidal composition of the invention, or an application mixture thereof.

The present invention further provides, the invention comprises a method for controlling insect activity, the method comprising applying to the plant an insecticidally effective compound of Formula (B), or a salt or solvate thereof.

The present invention further provides, the invention comprises a method for controlling fungal activity, the method comprising applying to the plant an insecticidally effective compound of Formula (B), or a salt or solvate thereof.

The present invention further provides, the invention comprises a method for regulating the growth of a crop plant, the method comprising applying to the plant an an effective compound of Formula (B), or a salt or solvate thereof.

Methods for Controlling the Growth of an Auxin-Susceptible Plant

In a fourth aspect, the invention comprises a method for suppressing the growth of an auxin-susceptible plant, the method comprising applying to the plant a herbicidally effective amount of a compound of Formula (1), or a salt or solvate thereof.

In a preferred form, the method comprises applying to the plant a herbicidally effective amount of a herbicidal composition containing a compound of Formula (1), or a salt or solvate thereof.

In a preferred form, method comprises applying to the plant a herbicidally effective amount of a herbicidal composition of the invention, as described above.

In a specific form of the invention, the herbicidal composition further comprises a herbicidally effective amount of a protoporphyrinogen inhibitor.

Some herbicidal compositions of the invention are preferably diluted to form an application mixture before being applied to the plant. Application mixtures may be prepared by dissolving the herbicidal composition in water or other suitable solvent or by suitable dilution of a concentrate herbicidal composition and applying to the foliage of unwanted plants by methods known in the art.

According to anyone of the invention embodiments, the methods of controlling the growth of auxin-susceptible plants comprise the steps of: (a) preparing an aqueous herbicidal application mixture by diluting with water a herbicidal composition concentrate of any of the herbicidal composition concentrates disclosed in this application; and (b) applying a herbicidally effective amount of the application mixture to the auxin-susceptible plants.

The herbicidal compositions of the invention, or application mixtures thereof, can be applied before planting, at planting, pre-emergence, or post-emergence to crop plants, such as in a field of the crop plants to control weeds in a field of the crop plants, depending on the particular herbicide salt and crop plant.

In a further embodiment, the composition according to the invention can be applied by treating seed. The treatment of seed comprises essentially all procedures familiar to the person skilled in the art (seed dressing, seed coating, seed dusting, seed soaking, seed film coating, seed multilayer coating, seed encrusting, seed dripping and seed pelleting) based on the compositions according to the invention. Here, the herbicidal compositions can be applied diluted or undiluted.

The term seed comprises seed of all types, such as, for example, corns, seeds, fruits, tubers, seedlings and similar forms. Here, preferably, the term seed describes corns and seeds.

The present invention further provides a method for treating an agricultural field comprising spraying the field with a herbicidal composition of the invention, or an application mixture thereof.

The auxin-susceptible plant may be a weed or a crop plant.

Crop plants include, for example, vegetable crops, grain crops, flowers, orchard crops, trees, and root crops.

In a preferred form of the method of the invention, herbicidal compositions of the invention or application mixtures thereof are applied to the foliage of crop plants and/or weed plants in the proximity of crop plants.

In one form of the invention, the weed plant is resistant to non-selective herbicides. In one form of the invention, the weed plant is resistant to glyphosate.

Crop plants include hybrids, in-breeds, and transgenic or genetically modified plants having specific traits or combinations of traits including, without limitation, herbicide tolerance (e.g., tolerant to carboxylic acid herbicides, such as auxin herbicides, or other herbicides), Bacillus thuringiensis (Bt), high oil, high lysine, high starch, nutritional density, and drought resistance.

In one form of the invention, the crop plant is selected from the group consisting of: corn, peanuts, potatoes, soybeans, canola, alfalfa, sugarcane, sugar beets, peanuts, grain sorghum, field beans, rice, sunflowers, wheat and cotton.

Crop plants that have been modified by breeding, mutagenesis or genetic engineering, e.g., have been rendered tolerant to applications of specific classes of herbicides are particularly useful with the compositions according to the invention.

In particular, tolerance to classes of herbicides has been developed such as auxin herbicides such as dicamba and 2,4-D; bleacher herbicides such as hydroxyphenylpyruvate dioxygenase (HPPD) inhibitors or phytoene desaturase (PDS) inhibitors; acetolactate synthase (ALS) inhibitors such as sulfonyl ureas or imidazolinones; enolpyruvyl shikimate 3-phosphate synthase (EPSP) inhibitors such as glyphosate; glutamine synthetase (GS) inhibitors such as glufosinate; protoporphyrinogen-IX oxidase (PPO) inhibitors; lipid biosynthesis inhibitors such as acetyl CoA carboxylase (ACCase) inhibitors; or oxynil (i.e. bromoxynil or ioxynil) herbicides as a result of conventional methods of breeding or genetic engineering.

These herbicide resistance technologies are, for example, described in Pest Management Science 61, 2005, 246; 61, 2005, 258; 61, 2005, 277; 61, 2005, 269; 61, 2005, 286; 64, 2008, 326; 64, 2008, 332; Weed Science 57, 2009, 108; Australian Journal of Agricultural Research 58, 2007, 708; Science 316, 2007, 1185; and references quoted therein, each herein incorporated by reference.

Further examples of these herbicide resistance technologies are described in US 2008/0028482, US2009/0029891, WO 2007/143690, WO 2010/080829, U.S. Pat. Nos. 6,307,129, 7,022,896, US 2008/0015110, U.S. Pat. Nos. 7,632,985, 7,105,724, and 7,381,861, each herein incorporated by reference.

In a preferred form of the invention, the crop plant is auxin herbicide tolerant.

In a preferred form of the invention, the crop plant is resistant to herbicides having the formula

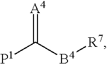

wherein:
P¹ is selected from the group consisting of;

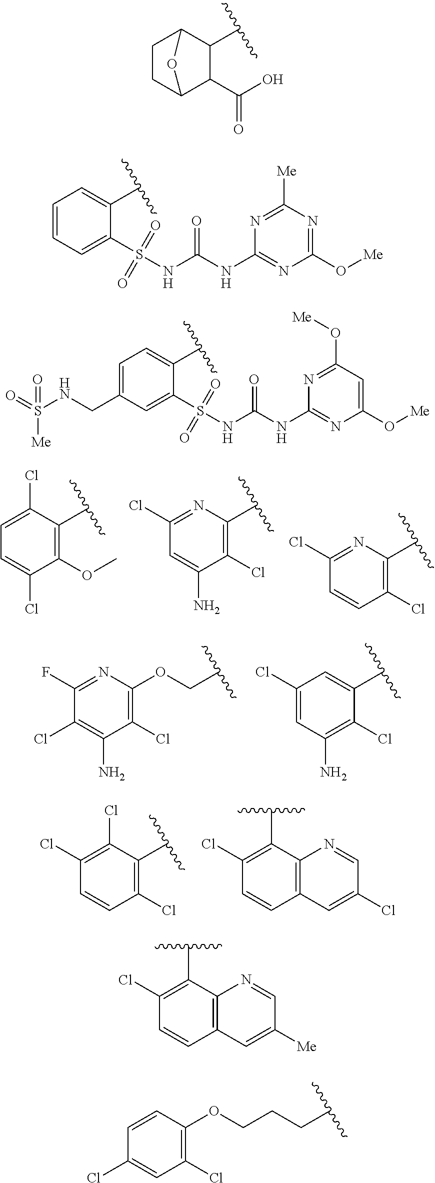

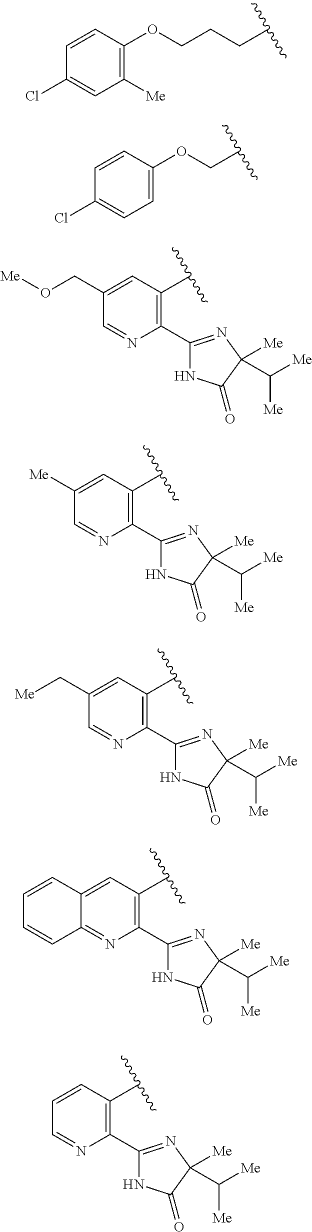

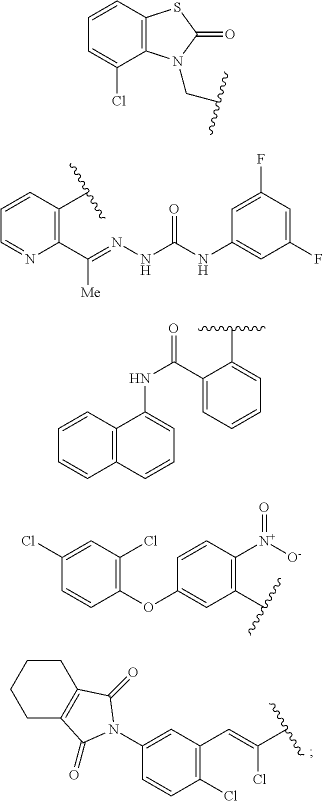 [SMILES: *Cn1c(=O)sc2cccc(Cl)c21 ; *c1cccnc1/C(C)=N/NC(=O)Nc1cc(F)cc(F)c1 ; *c1ccccc1C(=O)Nc1cccc2ccccc12 ; *c1cc(Oc2ccc(Cl)cc2Cl)ccc1[N+](=O)[O-] ; */C(Cl)=C/c1cc(N2C(=O)C3=C(CCCC3)C2=O)ccc1Cl]

A⁴ is O;
B⁴ is O or S;
R⁷ is H, or C₁-C₂ alkyl.

The soil bacterium Pseudomonas maltophilia (strain DI-6) converts dicamba to 3,6-dichlorosalicylic acid (3,6-DCSA), which is adsorbed to soil much more strongly than is dicamba, but lacks herbicidal activity.

The enzymes responsible for this first breakdown step is a three-component system called dicamba O-demethylase. Monsanto recently incorporated one component of the three enzymes into the genome of soybean, cotton, and other broadleaf crop plants, making them resistant to dicamba.

In a preferred form of the invention, the crop plant is dicamba tolerant.

In a specific form, the invention comprises a method for suppressing the growth of an auxin-susceptible weed in the proximity of a dicamba resistant crop, the method comprising applying to the weed a herbicidally effective amount of a herbicidal composition comprising compound of Formula (A). In one form of the invention, the weed is resistant to non-selective herbicides. In one form of the invention, the weed is resistant to glyphosate.

In a preferred form of the invention, the crop plant is modified by introduction of the dmo gene from Stenotrophomonas maltophilia strain DI-6.

In a preferred form of the invention, the crop plant is a cotton plant Gossypium hirsutum L. selected from the group consisting of:
a. COT102 x MON15985 x MON88913 x MON88701 (code SYN-IR1Ø2-7 x MON-15985-7 x MON-88913-8 x MON 887Ø1-3);
b. MON88701 (code MON 887Ø1-3);
c. MON88701 x MON88913 (code MON 887Ø1-3 x MON-88913-8); and
d. MON88701 x MON88913 x MON15985 (code MON 887Ø1-3 x MON-88913-8 x MON-15985-7).

In a preferred form of the invention, the crop plant is a maize plant Zea mays L. selected from the group consisting of:
a. MON87419 (code MON87419-8);
b. MON87427 x MON89034 x MIR162 x MON87419 x NK603 (code MON-87427-7 x MON-89Ø34-3 x SYN-IR162-4 x MON87419-8 x MON-ØØ6Ø3-6);
c. MON87427 x MON89034 x MON810 x MIR162 x MON87411 x MON87419 (code MON-87427-7 x MON-89Ø34-3 x MON-ØØ81Ø-6 x SYN-IR162-4 x MON-87411-9 x MON87419-8);
d. MON87427 x MON87419 x NK603 (code MON-87427-7 x MON87419-8 x MON-ØØ6Ø3-6); and
e. MON87427 x MON89034 x TC1507 x MON87411 x 59122 x MON87419 (code MON-87427-7 x MON-89Ø34-3 x DAS-Ø15Ø7-1 x MON-87411-9 x DAS-59122-7 x MON87419-8).

In a preferred form of the invention, the crop plant is a soybean plant Glycine max L. selected from the group consisting of:
a. DP305423 x MON87708 (code DP-3Ø5423-1 x MON-877Ø8-9);
b. DP305423 x MON87708 x MON89788 (code DP-3Ø5423-1 x MON-877Ø8-9 x MON-89788-1);
c. MON87705 x MON87708 (code MON-877Ø5-6 x MON-877Ø8-9)
d. MON87705 x MON87708 x MON89788 (code MON-877Ø5-6 x MON-877Ø8-9 x MON-89788-1)
e. MON87708 (code MON-877Ø8-9);
f. MON87708 x MON89788 (code MON-877Ø8-9 x MON-89788-1);
g. MON87708 x MON89788 x A5547-127 (code MON-877Ø8-9 x MON-89788-1 x ACS-GMØØ6-4); and
h. MON87751 x MON87701 x MON87708 x MON89788 (code MON-87751-7 x MON-877Ø1-2 x MON87708 x MON89788).

The codes referenced in the preceding paragraphs are those utilised by the International Service for the Acquisition of Agri-Biotech Applications (ISAAA).

In a specific form, the invention comprises a method for suppressing the growth of an auxin-susceptible weed in the proximity of a dicamba and protoporphyrinogen oxidase inhibitor resistant crop, the method comprising applying to the weed a herbicidally effective amount of a herbicidal composition comprising compound of Formula (B1), or a salt or solvate thereof, and a protoporphyrinogen oxidase inhibitor. In one form of the invention, the weed is resistant to non-selective herbicides.

Methods for generating a protoporphyrinogen oxidase inhibitor resistant crop may be found in, inter alia, US patent application 2017/0058290 and U.S. Pat. Nos. 10,041,087, 5,939,602 and 5,767,373, the contents of which are incorporated by reference.

The rates of application of the active compound are from 0.0001 to 3.0, preferably 0.01 to 1.0 kg/ha of active substance (a.s.), depending on the control target, the season, the target plants and the growth stage.

Where the compositions according to the invention are applied to the plants by spraying the leaves, the application can be carried out using, for example, water as carrier by customary spraying techniques using spray liquor amounts of from about 100 to 1000 l/ha (for example from 300 to 400 l/ha). The herbicidal compositions may also be applied by the low-volume or the ultra-low-volume method, or in the form of microgranules.

To treat the seed, the pesticides are generally employed in amounts of from 0.001 to 10 kg per 100 kg of seed.

The herbicidal composition can be applied to the foliage of a plant or plants at an application rate sufficient to give a commercially acceptable rate of weed control. This application rate is usually expressed as amount of herbicide per unit area treated, e.g., grams active substance per hectare (g a.s./ha).

Depending on plant species and growing conditions, the time required to achieve a commercially acceptable rate of weed control can be as short as a week or as long as three weeks, four weeks, or 30 days.

Moreover, it may be advantageous to apply the compositions of the present invention on their own or jointly in combination with other crop protection agents, for example with agents for controlling pests or phytopathogenic fungi or bacteria or with groups of active compounds which regulate growth.

The herbicidal compositions of the present invention may be used in conjunction with agents that convert herbicide resistant weeds to herbicide susceptible weeds, such as RNAi. In a specific form of the invention, the herbicidal compositions of the present invention may be used in conjunction with agents that convert auxin-resistant weeds to auxin-susceptible weeds, such as RNAi. See, for example U.S. Pat. No. 9,121,022—Sammons, R. D., Ivashutu, S., Liu, H., Wang, D., Feng, P. C. C., Kouranov, A. Y., and Andersen, S. E. 2015. Method for controlling herbicide-resistant plants.

General

Throughout this specification, unless the context requires otherwise, the phrase “compounds of the invention” includes salts and solvates of the compounds of Formula (A) and/or (B), and Formula (A1) and/or (1) as appropriate.

As used in this application and unless otherwise indicated, the terms “herbicide” and “herbicide composition” refers to a mixture that is produced, sold, or used in a field in order to kill or otherwise inhibit unwanted plants such as, but not limited to, deleterious or annoying weeds, broadleaf plants, grasses, and sedges; and can be used for crop protection, edifice protection or turf protection. The terms “herbicide” and “herbicide composition” includes the end-use herbicidal product. This composition can be a pure compound, a solution of chemical compounds, a mixture of chemical compounds, an emulsion, a suspension, a solid-liquid mixture, or a liquid-liquid mixture. The terms “herbicide” and “herbicide composition” also refer to the product that passes through the commercial channels from the manufacturer to the ultimate end user who can either apply the herbicide to the affected field as sold, as an application mixture and/or mix it with other excipients.

The term “weed” means and includes any plant which grows where it is not wanted, including volunteer crop plants or insecticide resistant plants.

The term “effective”, when used with respect to an herbicide, or the phrase “herbicidally effective amount” means an amount necessary to produce an observable herbicidal effect on unwanted plant growth, including one or more of the effects of necrosis, death, growth inhibition, reproduction inhibition, inhibition of proliferation, and removal, destruction, or otherwise diminishing the occurrence and activity of unwanted plants.

The term “controlling”, as in “controlling plant growth”, means producing an observable herbicidal effect on the plant, including one or of the effects of necrosis, death, growth inhibition, reproduction inhibition, inhibition of proliferation, and removal, destruction, or otherwise diminishing the occurrence and activity of unwanted plants.

The phrase “agriculturally acceptable” as used herein refers to a component that is not unacceptably damaging to a plant or its environment, and/or not unsafe to the user or others that may be exposed to the material when used as described herein

The phrase “herbicidal activity” as used herein in respect to the properties of the compounds of the invention, means that the compound of the invention inhibits the normal growth of a plant. Activity such as inducing chlorosis is included within the definition of herbicidal activity, as it affects the normal growth of a plant. In some instances, the herbicidal activity of the compounds of the invention is such that it causes the death of the plant, but the term herbicidal activity as used in the specification is not intended to be limited to this outcome.

Throughout this specification, unless the context requires otherwise, the word “comprise”, or variations such as “comprises” or “comprising”, will be understood to imply the inclusion of a stated integer or group of integers but not the exclusion of any other integer or group of integers.

The present invention includes and expressly contemplates any and all combinations of embodiments, features, characteristics, parameters, and/or ranges disclosed herein. That is, the invention may be defined by any combination of embodiments, features, characteristics, parameters, and/or ranges mentioned herein.

It is contemplated that any ingredient, component, or step that is not specifically named or identified as part of the invention may be explicitly excluded by at least some embodiments of the invention.

While attempts have been made to be precise, the numerical values and ranges described herein should be considered to be approximations (even when not qualified by the term “about”). These values and ranges may vary from their stated numbers depending upon the desired properties sought to be obtained by the present invention as well as the variations resulting from the standard deviation found in the measuring techniques. Moreover, the ranges described herein are intended and specifically contemplated to include all sub-ranges and values within the stated ranges. For example, a range of 50 to 100 is intended to describe and include all values within the range including sub-ranges such as 60 to 90 and 70 to 80.

Any two numbers of the same property or parameter reported in the working examples may define a range. Those numbers may be rounded off to the nearest thousandth, hundredth, tenth, whole number, ten, hundred, or thousand to define the range.

The content of all documents cited herein, including patents as well as non-patent literature, is hereby incorporated by reference in their entirety. To the extent that any incorporated subject matter contradicts with any disclosure herein, the disclosure herein shall take precedence over the incorporated content. Should the disclosure of any patents, patent applications, and publications which are incorporated herein by reference conflict with the description of the present application to the extent that it may render a term unclear, the present description shall take precedence.

### Examples

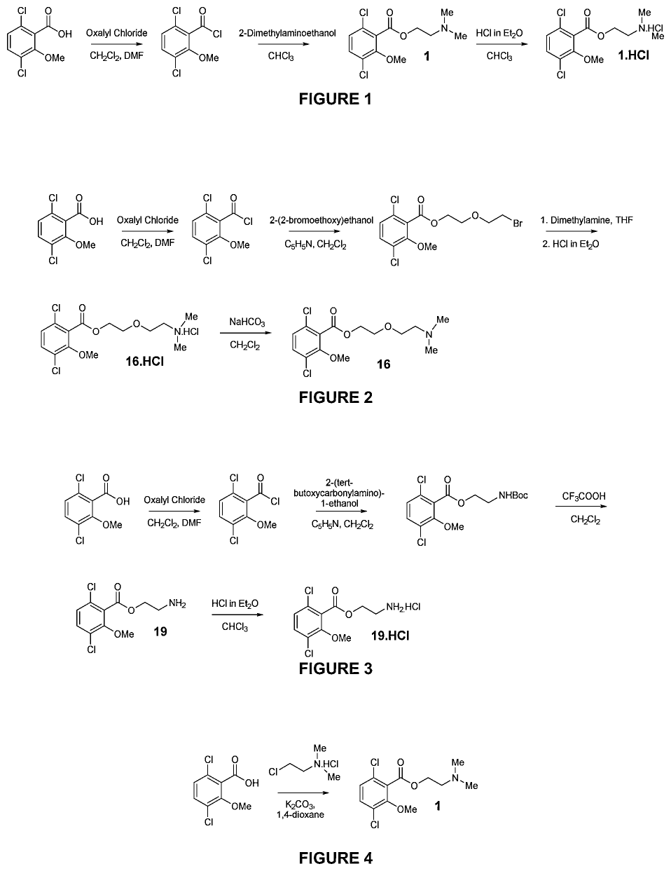

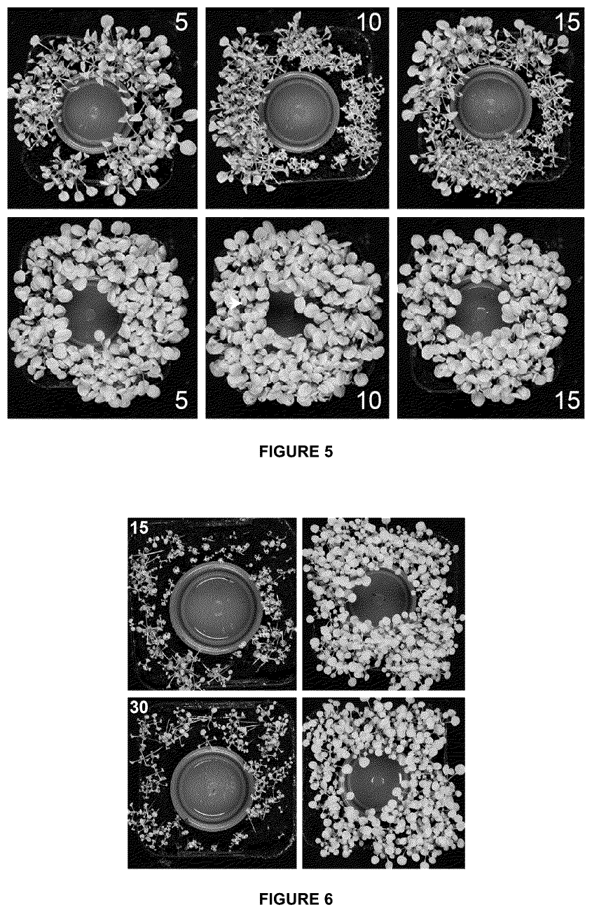

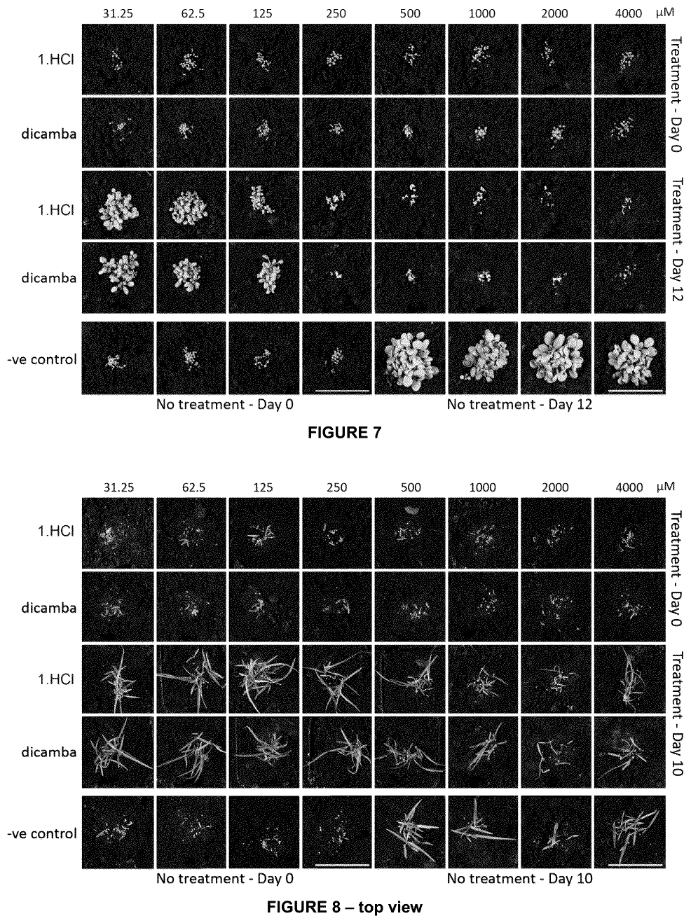

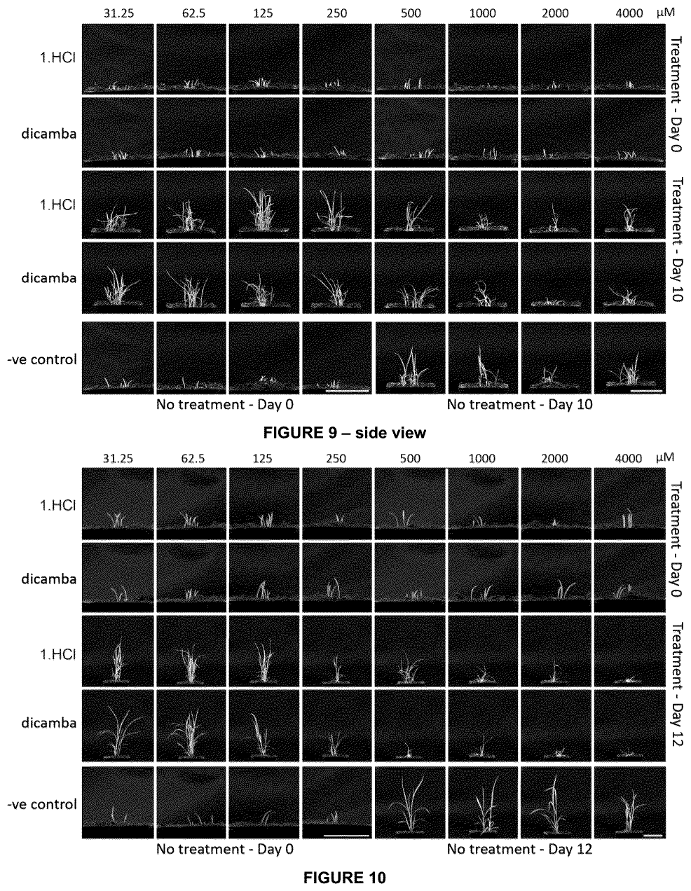

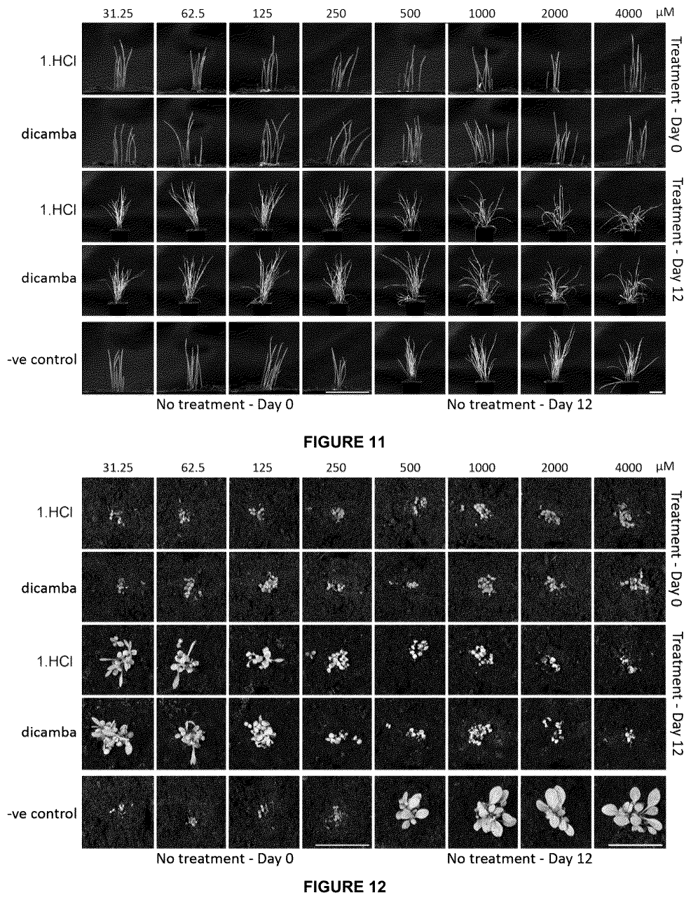

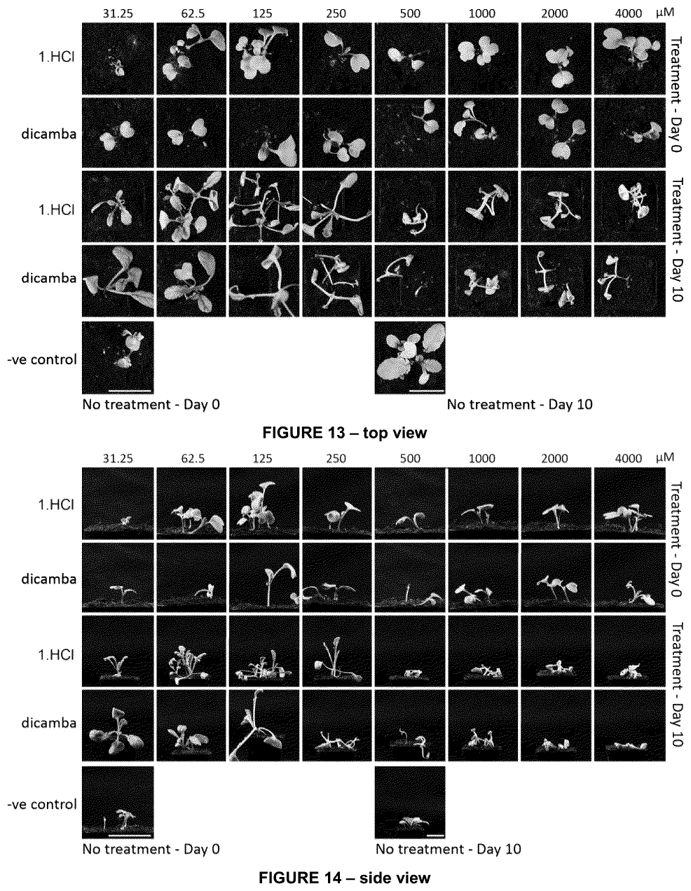

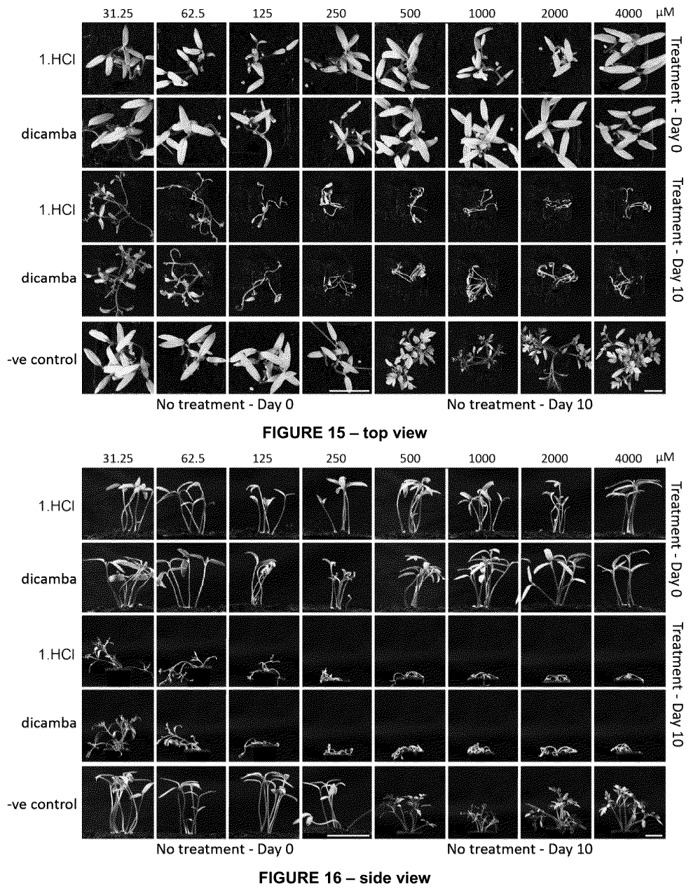

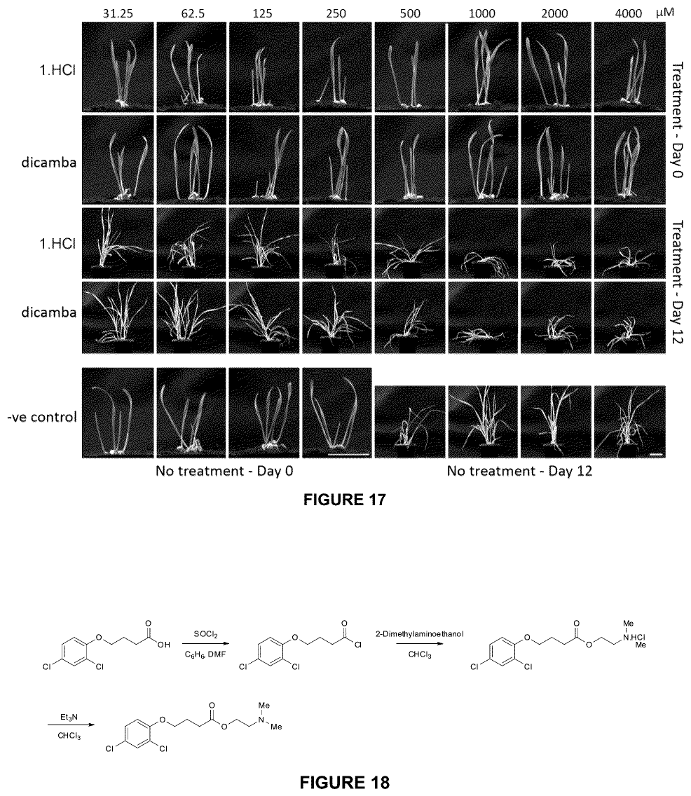

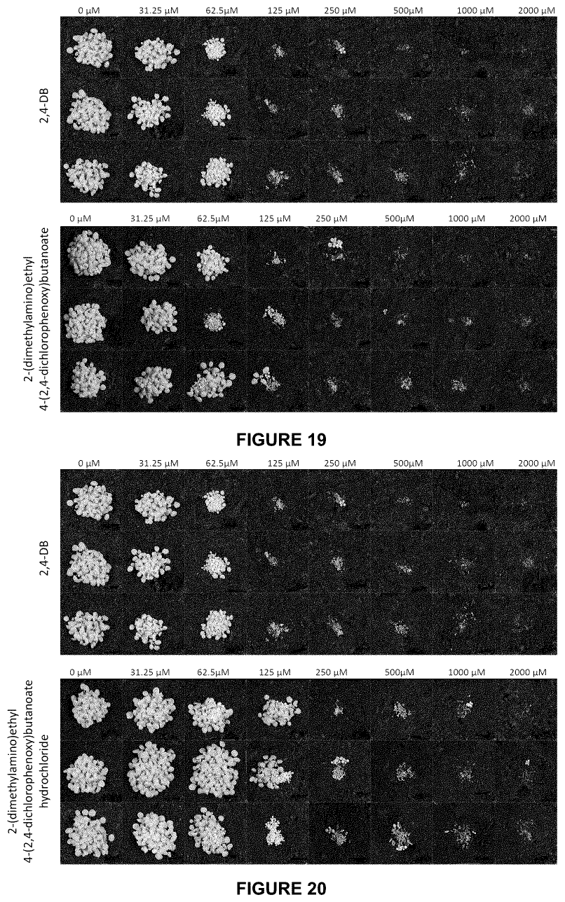

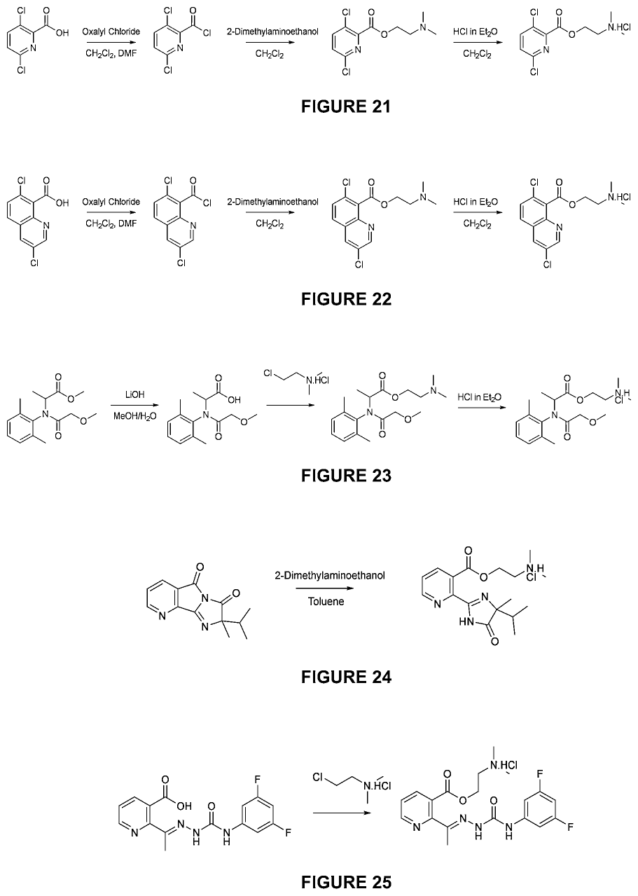

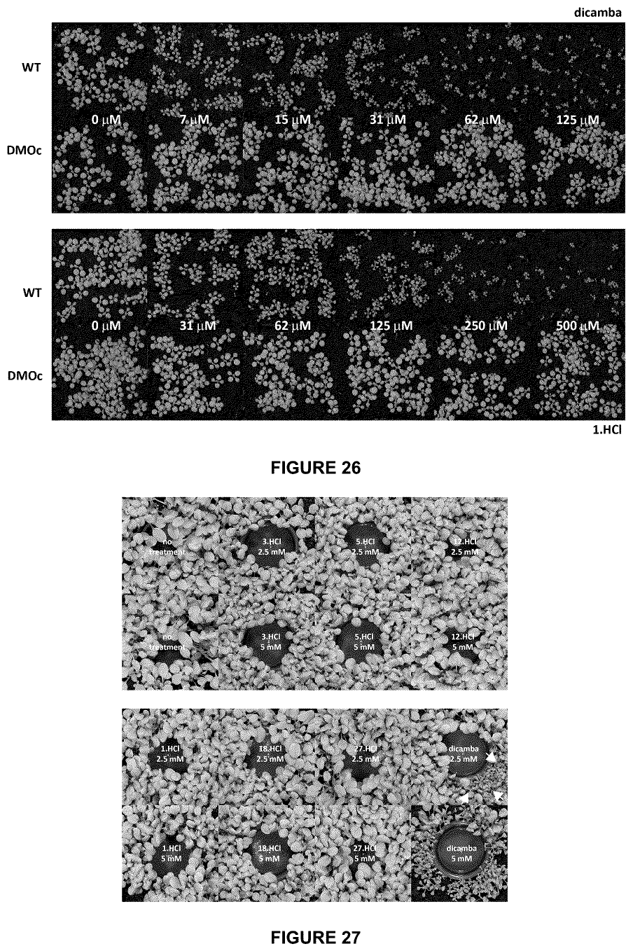

The present invention will now be described with reference to a series of examples. The description of the examples should not be understood to be limiting the generality of the preceding description of the invention. The Figures referenced in the description of the Examples are as follows:
a. FIG. 1 is a synthetic scheme for a compound of the invention being 2-(dimethylamino)ethyl 3,6-dichloro-2-methoxybenzoate hydrochloride;
b. FIG. 2 provides a scheme for the synthesis of 2-(2-(dimethylamino)ethoxy)ethyl 3,6-dichloro-2-methoxybenzoate hydrochloride from the readily available commercial pesticide dicamba;
c. FIG. 3 provides a scheme for the synthesis of 2-aminoethyl 3,6-dichloro-2-methoxybenzoate hydrochloride from the readily available commercial pesticide dicamba;
d. FIG. 4 is a scheme for the synthesis of 2-(dimethylamino)ethyl 3,6-dichloro-2-methoxybenzoate from the readily available commercial pesticide dicamba;
e. FIG. 5 is series of photographs of Arabidopsis thaliana illustrating the different volatility characteristics of the compound of the invention and the known auxin herbicide dicamba at one of three concentrations (5, 10 or 15 mM);
f. FIG. 6 is series of photographs of Arabidopsis thaliana illustrating the different volatility characteristics of the compound of the invention and the known auxin herbicide dicamba at one of two concentrations (15 mM and 30 mM);
g. FIG. 7 is series of photographs of Arabidopsis thaliana (top view) illustrating the comparative efficacy of the compound of the invention and the known auxin herbicide dicamba at a range of concentrations;
h. FIG. 8 is series of photographs of Cynodon dactylon (top view) illustrating the comparative efficacy of the compound of the invention and the known auxin herbicide dicamba at a range of concentrations;
i. FIG. 9 is series of photographs of Cynodon dactylon (side view) illustrating the comparative efficacy of the compound of the invention and the known auxin herbicide dicamba at a range of concentrations;
j. FIG. 10 is series of photographs of Eragrostis tef illustrating the comparative efficacy of the compound of the invention and the known auxin herbicide dicamba at a range of concentrations;
k. FIG. 11 is series of photographs of Lolium rigidum illustrating the comparative efficacy of the compound of the invention and the known auxin herbicide dicamba at a range of concentrations;
l. FIG. 12 series of photographs of Ratibida columnifera illustrating the comparative efficacy of the compound of the invention and the known auxin herbicide dicamba at a range of concentrations;
m. FIG. 13 is series of photographs of Raphanus raphanistrum (top view) illustrating the comparative efficacy of the compound of the invention and the known auxin herbicide dicamba at a range of concentrations;
n. FIG. 14 is series of photographs of Raphanus raphanistrum (side view) illustrating the comparative efficacy of the compound of the invention and the known auxin herbicide dicamba at a range of concentrations;
o. FIG. 15 is series of photographs of Solanum lycopersicum (top view) illustrating the comparative efficacy of the compound of the invention and the known auxin herbicide dicamba at a range of concentrations;
p. FIG. 16 is series of photographs of Solanum lycopersicum (side view) illustrating the comparative efficacy of the compound of the invention and the known auxin herbicide dicamba at a range of concentrations;
q. FIG. 17 is series of photographs of Triticum aestivum (side view) illustrating the comparative efficacy of the compound of the invention and the known auxin herbicide dicamba at a range of concentrations; and
r. FIG. 18 is a synthetic scheme for 2-(dimethylamino)ethyl 4-(2,4-dichlorophenoxy)butanoate and 2-(dimethylamino)ethyl 4-(2,4-dichlorophenoxy)butanoate hydrochloride respectively;
s. FIG. 19 is a series of photographs of Arabidopsis thaliana (top view) illustrating the comparative efficacy of 2-(dimethylamino)ethyl 4-(2,4-dichlorophenoxy)butanoate and the known auxin herbicide 2,4-DB at a range of concentrations;
t. FIG. 20 is a series of photographs of Arabidopsis thaliana (top view) illustrating the comparative efficacy of the 2-(dimethylamino)ethyl 4-(2,4-dichlorophenoxy)butanoate hydrochloride and the known auxin herbicide 2,4-DB at a range of concentrations;
u. FIG. 21 represents the synthesis of 3,6-dichloropyridine-2-carboxylic acid 2-(dimethylamino)ethyl ester and 3,6-dichloropyridine-2-carboxylic acid 2-(dimethylamino)ethyl ester hydrochloride;
v. FIG. 22 represents the synthesis of 3,7-dichloroquinoline-8-carboxylic acid 2-(dimethylamino)ethyl ester and 3,7-dichloroquinoline-8-carboxylic acid 2-(dimethylamino)ethyl ester hydrochloride;
w. FIG. 23 represents the synthesis of N-(2,6-dimethylphenyl)-N-(methoxyacetyl)-DL-alanine 2-(dimethylamino)ethyl ester and N-(2,6-dimethylphenyl)-N-(methoxyacetyl)-DL-alanine 2-(dimethylamino)ethyl ester hydrochloride;
x. FIG. 24 represents the synthesis of 2-(Dimethylamino)ethyl 2-[4,5-dihydro-4-methyl-4-(1-methylethyl)-5-oxo-1H-imidazol-2-yl]-3-pyridinecarboxylate hydrochloride;
y. FIG. 25 represents the synthesis of 2-(Dimethylamino)ethyl 2-[1-[2-[[(3-fluorophenyl)amino]carbonyl]hydrazinylidene]ethyl]-3-pyridinecarboxylate hydrochloride;
z. FIG. 26 represents a series of photographs of Arabidopsis thaliana illustrating the compatibility of both dicamba and 1·HCl with the commercial dicamba tolerance trait. Plants are wild type (WT) or a transgenic line expressing a dicamba monooxygenase (DMOc) gene. A range of concentrations were used;
aa. FIG. 27 represents is series of photographs of Arabidopsis thaliana illustrating the volatility characteristics of 1·HCl; (bottom left), the known auxin herbicide dicamba (bottom right) as well as 3·HCl, 5·HCl, 12·HCl, 18·HCl, and 27·HCl. All are shown at two different concentrations (2.5 or 5 mM).

Synthesis

FIG. 1 provides a scheme for the synthesis of 2-(dimethylamino)ethyl 3,6-dichloro-2-methoxybenzoate hydrochloride 1·HCl from the readily available commercial pesticide dicamba.

### 3,6-dichloro-2-methoxybenzoyl chloride

To a solution of 3,6-dichloro-2-methoxybenzoic acid (Dicamba) (500 mg, 2.3 mmol) in CH₂Cl₂ (10 ml) was added DMF (3 drops) and oxalyl chloride (0.25 ml, 3.0 mmol) at room temperature and stirred for 2 h at this temperature. The solution was then concentrated and the resultant residue co-evaporated with toluene (3×10 ml) to give 3,6-dichloro-2-methoxybenzoyl chloride as a pale yellow oil (530 mg).

### 2-(dimethylamino)ethyl 3,6-dichloro-2-methoxybenzoate 1

To a solution of 2-dimethylaminoethanol (0.45 ml, 4.5 mmol) in CHCl₃ (7.5 ml) was added 3,6-dichloro-2-methoxybenzoyl chloride (530 mg, 2.2 mmol) in CHCl₃ (7.5 ml) at 0° C. and the resultant solution stirred at room temperature for 24 h. The mixture was then transferred to a separating funnel and washed with sat. NaHCO₃ (15 ml), brine (15 ml), dried (MgSO₄), filtered and concentrated. The resultant residue was then purified by flash column chromatography (MeOH:CHCl₃ 3:97) to obtain 2-(dimethylamino)ethyl 3,6-dichloro-2-methoxybenzoate 1 as a pale yellow oil (540 mg, 82%, over two steps). R_{f}=0.30 (MeOH:CHCl₃ 1:24). ¹H NMR (500 MHz, d₆-DMSO): δ 7.66 (d, J=8.8 Hz, 1H), 7.39 (d, J=8.8 Hz, 1H), 4.39 (t, J=5.6 Hz, 2H), 3.84 (s, 3H), 2.58 (t, J=5.6 Hz, 2H), 2.18 (s, 6H); ¹³C NMR (125.8 MHz, d₆-DMSO): δ 163.7, 153.0, 132.4, 129.9, 128.5, 126.2, 126.0, 63.7, 62.1, 56.8, 45.2.

### 2-(dimethylamino)ethyl 3,6-dichloro-2-methoxybenzoate hydrochloride 1·HCl

To a solution of 2-(dimethylamino)ethyl 3,6-dichloro-2-methoxybenzoate (500 mg, 1.7 mmol) in CHCl₃ (15 ml) was added saturated hydrogen chloride in ether until the resultant solution was pH=˜2 (˜1 ml) and the resultant solution was stirred at room temperature for 10 min. Toluene (10 ml) was then added to the solution and the solution was then concentrated to give 2-(dimethylamino)ethyl 3,6-dichloro-2-methoxybenzoate hydrochloride 1·HCl as a white solid (560 mg, quant.). ¹H NMR (500 MHz, d₆-DMSO): δ 10.89 (br s, 1H), 7.70 (d, J=8.7 Hz, 1H), 7.43 (d, J=8.7 Hz, 1H), 4.73 (t, J=5.2 Hz, 2H), 3.86 (s, 3H), 3.49 (t, J=5.2 Hz, 2H), 2.79 (s, 6H); ¹³C NMR (125.8 MHz, d₆-DMSO): δ 163.3, 153.2, 132.9, 129.1, 128.6, 126.4, 126.1, 62.4, 60.7, 54.3, 42.5.

As would be understood by a person skilled in the art, the synthetic scheme presented above can be readily adapted to synthesise other compounds of Formula (A). For example, replacement of 2-methylaminoethanol with the appropriate alcohol, amine or thiol, or by treatment with Lawesson's reagent (pre- or post-esterification). (See, for example, Synlett 1994; 1994(4): 251-252, the contents of which are hereby incorporated by reference).

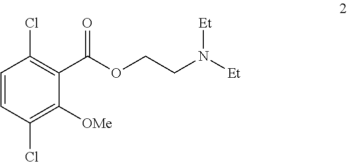 [SMILES: CCN(CC)CCOC(=O)c1c(Cl)ccc(Cl)c1OC]

### 2-(diethylamino)ethyl 3,6-dichloro-2-methoxybenzoate 2

Prepared as per 2-(dimethylamino)ethyl 3,6-dichloro-2-methoxybenzoate except 2-(diethylamino)ethanol was used. ¹H NMR (400 MHz, CDCl₃): δ 7.33 (d, J=8.7 Hz, 1H), 7.10 (d, J=8.7 Hz, 1H), 4.43 (t, J=6.5 Hz, 2H), 3.91 (s, 3H), 2.83 (t, J=6.5 Hz, 2H), 2.60 (q, J=7.1 Hz, 4H), 1.03 (t, J=7.1 Hz, 6H); ¹³C NMR (100.6 MHz, CDCl₃): δ 164.7, 154.0, 131.8, 130.7, 129.7, 126.8, 125.9, 64.4, 62.4, 51.2, 47.6, 12.0.

### 2-(diethylamino)ethyl 3,6-dichloro-2-methoxybenzoate hydrochloride 2·HCl

Prepared as per 2-(dimethylamino)ethyl 3,6-dichloro-2-methoxybenzoate hydrochloride except 2-(diethylamino)ethyl 3,6-dichloro-2-methoxybenzoate was used. ¹H NMR (500 MHz, d₆-DMSO): δ 10.75 (br s, 1H), 7.70 (d, J=8.7 Hz, 1H), 7.43 (d, J=8.7 Hz, 1H), 4.75 (t, J=5.3 Hz, 2H), 3.86 (s, 3H), 3.53-3.44 (m, 2H), 3.22-3.09 (m, 4H), 1.21 (t, J=7.2 Hz, 6H); ¹³C NMR (125.8 MHz, d₆-DMSO): δ 163.4, 153.2, 133.0, 129.1, 128.6, 126.4, 126.2, 62.4, 60.6, 48.9, 46.8, 8.5.

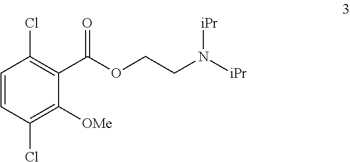 [SMILES: COc1c(Cl)ccc(Cl)c1C(=O)OCCN(C(C)C)C(C)C]

### 2-(dipropan-2-ylamino)ethyl 3,6-dichloro-2-methoxybenzoate 3

Prepared as per 2-(dimethylamino)ethyl 3,6-dichloro-2-methoxybenzoate except 2-(diisopropylamino)ethanol was used. ¹H NMR (400 MHz, CDCl₃): δ 7.34 (d, J=8.7 Hz, 1H), 7.11 (d, J=8.7 Hz, 1H), 4.28 (t, J=7.7 Hz, 2H), 3.92 (s, 3H), 3.03 (sept, J=6.5 Hz, 2H), 2.78 (t, J=7.7 Hz, 2H), 1.03 (d, J=6.5 Hz, 12H); ¹³C NMR (100.6 MHz, CDCl₃): δ 164.7, 153.9, 131.8, 130.8, 129.7, 126.9, 126.0, 67.0, 62.4, 49.6, 43.5, 21.0.

### 2-(dipropan-2-ylamino)ethyl 3,6-dichloro-2-methoxybenzoate hydrochloride 3·HCl

Prepared as per 2-(dimethylamino)ethyl 3,6-dichloro-2-methoxybenzoate hydrochloride except 2-(dipropan-2-ylamino)ethyl 3,6-dichloro-2-methoxybenzoate was used. ¹H NMR (400 MHz, d₆-DMSO): δ 9.97 (br s, 1H), 7.71 (d, J=8.7 Hz, 1H), 7.43 (d, J=8.7 Hz, 1H), 4.74 (t, J=5.9 Hz, 2H), 3.87 (s, 3H), 3.75-3.63 (m, 2H), 3.52-3.44 (m, 2H), 1.32 (d, J=6.6 Hz, 6H), 1.30 (d, J=6.6 Hz, 6H); ¹³C NMR (100 MHz, d₆-DMSO): δ 163.4, 153.2, 132.9, 129.0, 128.6, 126.4, 126.1, 79.2, 62.4, 61.7, 54.4, 44.3, 17.9, 16.6.

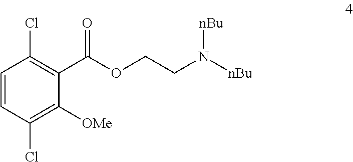

### 2-(dibutylamino)ethyl 3,6-dichloro-2-methoxybenzoate 4

Prepared as per 2-(dimethylamino)ethyl 3,6-dichloro-2-methoxybenzoate except 2-(dibutylamino)ethanol was used. ¹H NMR (400 MHz, CDCl₃): δ 7.34 (d, J=8.6 Hz, 1H), 7.10 (d, J=8.6 Hz, 1H), 4.41 (t, J=6.6 Hz, 2H), 3.91 (s, 3H), 2.82 (t, J=6.6 Hz, 2H), 2.52-2.44 (m, 4H), 1.49-1.36 (m, 4H), 1.36-1.22 (m, 4H), 0.89 (t, J=7.3 Hz, 6H); ¹³C NMR (100.6 MHz, CDCl₃): δ 164.7, 154.0, 131.8, 130.7, 129.7, 126.8, 125.9, 64.5, 62.4, 54.6, 52.3, 29.6, 20.7, 14.2.

### 2-(dibutylamino)ethyl 3,6-dichloro-2-methoxybenzoate hydrochloride 4·HCl

Prepared as per 2-(dimethylamino)ethyl 3,6-dichloro-2-methoxybenzoate hydrochloride except 2-(dibutylamino)ethyl 3,6-dichloro-2-methoxybenzoate was used. ¹H NMR (400 MHz, d₆-DMSO): δ 10.87 (br s, 1H), 7.70 (d, J=8.7 Hz, 1H), 7.43 (d, J=8.7 Hz, 1H), 4.80-4.73 (m, 2H), 3.86 (s, 3H), 3.52-3.48 (m, 2H), 3.10-3.02 (m, 4H), 1.69-1.55 (m, 4H), 1.32. 1.18 (m, 4H), 0.84 (t, J=7.3 Hz, 6H); ¹³C NMR (100.6 MHz, d₆-DMSO): δ 163.3, 153.2, 132.9, 129.1, 128.6, 126.4, 126.2, 62.4, 60.7, 52.3, 49.9, 24.8, 19.4, 13.4.

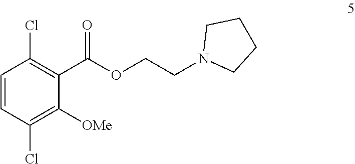 [SMILES: COc1c(Cl)ccc(Cl)c1C(=O)OCCN1CCCC1]

### 2-(pyrrolidin-1-yl)ethyl 3,6-dichloro-2-methoxybenzoate 5

Prepared as per 2-(dimethylamino)ethyl 3,6-dichloro-2-methoxybenzoate except 1-(2-hydroxyethyl)pyrrolidine was used. ¹H NMR (400 MHz, CDCl₃): δ 7.34 (d, J=8.7 Hz, 1H), 7.10 (d, J=8.7 Hz, 1H), 4.50 (t, J=6.1 Hz, 2H), 3.91 (s, 3H), 2.86 (t, J=6.1 Hz, 2H), 2.64-2.53 (m, 4H), 1.85-1.71 (m, 4H); ¹³C NMR (100.6 MHz, CDCl₃): δ 164.7, 154.0, 131.9, 130.7, 129.8, 126.8, 125.9, 65.1, 62.4, 54.6, 54.3, 23.7.

### 2-(pyrrolidin-1-yl)ethyl 3,6-dichloro-2-methoxybenzoate hydrochloride 5·HCl

Prepared as per 2-(dimethylamino)ethyl 3,6-dichloro-2-methoxybenzoate hydrochloride except 2-(pyrrolidin-1-yl)ethyl 3,6-dichloro-2-methoxybenzoate was used. ¹H NMR (400 MHz, d₆-DMSO): δ 11.15 (br s, 1H), 7.70 (d, J=8.7 Hz, 1H), 7.42 (d, J=8.7 Hz, 1H), 4.72 (d, J=5.3 Hz, 2H), 3.87 (s, 3H), 3.66-3.43 (m, 4H), 3.08-3.04 (m, 2H), 2.00-1.77 (m, 4H); ¹³C NMR (100.6 MHz, d₆-DMSO): δ 163.4, 153.2, 132.9, 129.2, 128.6, 126.4, 126.1, 62.4, 61.6, 53.4, 51.6, 22.6.

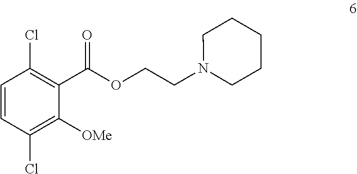 [SMILES: COc1c(Cl)ccc(Cl)c1C(=O)OCCN1CCCCC1]

### 2-(piperidin-1-yl)ethyl 3,6-dichloro-2-methoxybenzoate 6

Prepared as per 2-(dimethylamino)ethyl 3,6-dichloro-2-methoxybenzoate except 1-(2-hydroxyethyl)piperidine was used. ¹H NMR (400 MHz, CDCl₃): δ 7.34 (d, J=8.7 Hz, 1H), 7.10 (d, J=8.6 Hz, 1H), 4.50 (t, J=6.1 Hz, 2H), 3.92 (s, 3H), 2.72 (t, J=6.1 Hz, 2H), 2.50-2.43 (m, 4H), 1.63-1.53 (m, 4H), 1.48-1.38 (m, 2H); ¹³C NMR (100.6 MHz, CDCl₃): δ 164.7, 154.0, 131.9, 130.7, 129.8, 126.9, 125.9, 63.8, 62.4, 57.2, 54.9, 26.1, 24.3.

### 2-(piperidin-1-yl)ethyl 3,6-dichloro-2-methoxybenzoate hydrochloride 6·HCl

Prepared as per 2-(dimethylamino)ethyl 3,6-dichloro-2-methoxybenzoate hydrochloride except 2-(piperidin-1-yl)ethyl 3,6-dichloro-2-methoxybenzoate was used. ¹H NMR (400 MHz, d₆-DMSO): δ 10.86 (br s, 1H), 7.70 (d, J=8.7 Hz, 1H), 7.43 (d, J=8.7 Hz, 1H), 4.82-4.75 (m, 2H), 3.86 (s, 3H), 3.45 (s, 4H), 2.97-2.93 (m, 2H), 1.82-1.73 (m, 4H), 1.67 (s, 1H), 1.36 (s, 1H); ¹³C NMR (100.6 MHz, d₆-DMSO): δ 163.3, 153.2, 132.9, 129.1, 128.6, 126.4, 126.1, 62.4, 60.6, 53.8, 52.4, 22.4, 21.1.

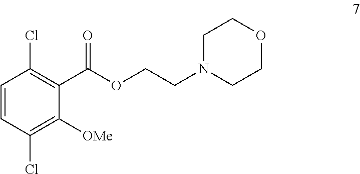 [SMILES: COc1c(Cl)ccc(Cl)c1C(=O)OCCN1CCOCC1]

### 2-(morpholin-4-yl)ethyl 3,6-dichloro-2-methoxybenzoate 7

Prepared as per 2-(dimethylamino)ethyl 3,6-dichloro-2-methoxybenzoate except 4-(2-hydroxyethyl)morpholine was used. ¹H NMR (400 MHz, CDCl₃): δ 7.35 (d, J=8.6 Hz, 1H), 7.11 (d, J=8.7 Hz, 1H), 4.50 (t, J=5.8 Hz, 2H), 3.91 (s, 3H), 3.70 (t, J=4.6 Hz, 4H), 2.75 (t, J=5.8 Hz, 2H), 2.54 (t, J=4.6 Hz, 4H); ¹³C NMR (100.6 MHz, CDCl₃): δ 164.6, 154.0, 132.0, 130.5, 129.7, 126.9, 126.0, 67.0, 63.3, 62.4, 56.9, 53.9.

### 2-(morpholin-4-yl)ethyl 3,6-dichloro-2-methoxybenzoate hydrochloride 7·HCl

Prepared as per 2-(dimethylamino)ethyl 3,6-dichloro-2-methoxybenzoate hydrochloride except 2-(morpholin-4-yl)ethyl 3,6-dichloro-2-methoxybenzoate was used. ¹H NMR (500 MHz, d₆-DMSO): δ 11.51 (br s, 1H), 7.69 (d, J=8.7 Hz, 1H), 7.42 (d, J=8.7 Hz, 1H), 4.82-4.76 (m, 2H), 4.03-3.72 (m, 7H), 3.55-3.51 (m, 2H), 3.34-3.01 (m, 4H); ¹³C NMR (125.8 MHz, d₆-DMSO): δ 163.4, 153.3, 133.0, 129.1, 128.7, 126.4, 126.2, 63.2, 62.4, 60.5, 54.0, 51.5.

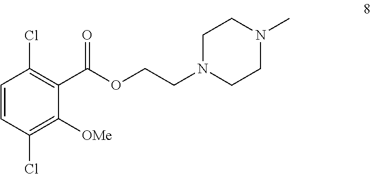 [SMILES: COc1c(Cl)ccc(Cl)c1C(=O)OCCN1CCN(C)CC1]

### 2-(4-methylpiperazin-1-yl)ethyl 3,6-dichloro-2-methoxybenzoate 8

Prepared as per 2-(dimethylamino)ethyl 3,6-dichloro-2-methoxybenzoate except 4-methylpiperazine-1-ethanol was used. ¹H NMR (400 MHz, CDCl₃): δ 7.34 (d, J=8.7 Hz, 1H), 7.11 (d, J=8.7 Hz, 1H), 4.49 (t, J=5.9 Hz, 2H), 3.91 (s, 3H), 2.76 (t, J=5.9 Hz, 2H), 2.70-2.31 (m, 8H), 2.27 (s, 3H); ¹³C NMR (100.6 MHz, CDCl₃): δ 164.6, 154.0, 131.9, 130.6, 129.8, 126.9, 125.9, 63.6, 62.4, 56.5, 55.2, 53.4, 46.2.

### 2-(4-methylpiperazin-1-yl)ethyl 3,6-dichloro-2-methoxybenzoate dihydrochloride 8·HCl

Prepared as per 2-(dimethylamino)ethyl 3,6-dichloro-2-methoxybenzoate hydrochloride except 2-(4-methylpiperazin-1-yl)ethyl 3,6-dichloro-2-methoxybenzoate was used. ¹H NMR (500 MHz, d₆-DMSO): δ 11.58 (br s, 2H), 7.69 (d, J=8.7 Hz, 1H), 7.41 (d, J=8.7 Hz, 1H), 4.75-4.67 (m, 2H), 3.87 (s, 3H), 3.73-3.14 (m, 10H), 2.79 (s, 3H); ¹³C NMR (125.8 MHz, d₆-DMSO): δ 163.3, 153.1, 132.8, 129.1, 128.6, 126.3, 126.0, 79.1, 62.3, 61.0, 53.6, 48.3, 41.6.

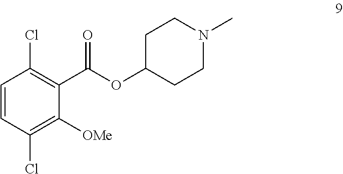 [SMILES: COc1c(Cl)ccc(Cl)c1C(=O)OC1CCN(C)CC1]

### 1-methylpiperidin-4-yl 3,6-dichloro-2-methoxybenzoate 9

Prepared as per 2-(dimethylamino)ethyl 3,6-dichloro-2-methoxybenzoate except N-methyl-4-piperidinol was used. ¹H NMR (400 MHz, CDCl₃): δ 7.34 (d, J=8.6 Hz, 1H), 7.10 (d, J=8.7 Hz, 1H), 5.17-5.06 (m, 1H), 3.90 (d, J=1.0 Hz, 3H), 2.73-2.66 (m, 2H), 2.34-2.23 (m, 5H), 2.11-2.00 (m, 2H), 1.93-1.80 (m, 2H); ¹³C NMR (100.6 MHz, CDCl₃): δ 164.1, 153.8, 131.8, 130.9, 129.7, 126.9, 126.0, 72.2, 62.3, 53.0, 46.3, 30.9.

### 1-methylpiperidin-4-yl 3,6-dichloro-2-methoxybenzoate hydrochloride 9·HCl

Prepared as per 2-(dimethylamino)ethyl 3,6-dichloro-2-methoxybenzoate hydrochloride except 1-methylpiperidin-4-yl 3,6-dichloro-2-methoxybenzoate was used. ¹H NMR (500 MHz, d₆-DMSO): δ 10.81 (s, 1H), 7.68 (d, J=8.7 Hz, 1H), 7.41 (d, J=8.7 Hz, 1H), 5.30-5.27 (m, 1H), 3.84 (s, 3H), 3.32-3.08 (m, 4H), 2.72 (s, 3H), 2.27-2.19 (m, 2H), 2.08-2.00 (m, 2H); ¹³C NMR (125.8 MHz, d₆-DMSO): δ 163.0, 153.1, 132.7, 129.6, 128.6, 126.5, 126.2, 69.5, 62.3, 51.1, 42.2, 27.1.

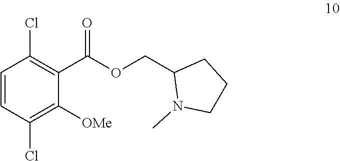 [SMILES: COc1c(Cl)ccc(Cl)c1C(=O)OCC1CCCN1C]

### (1-methylpyrrolidin-2-yl)methyl 3,6-dichloro-2-methoxybenzoate 10

Prepared as per 2-(dimethylamino)ethyl 3,6-dichloro-2-methoxybenzoate except 1-methyl-2-pyrrolidinemethanol was used. ¹H NMR (400 MHz, CDCl₃): δ 7.34 (d, J=8.7 Hz, 1H), 7.11 (d, J=8.7 Hz, 1H), 4.45 (dd, J=11.0, 4.9 Hz, 1H), 4.25 (dd, J=11.0, 6.1 Hz, 1H), 3.91 (s, 3H), 3.11-3.02 (m, 1H), 2.64-2.52 (m, 1H), 2.43 (s, 3H), 2.32-2.19 (m, 1H), 2.07-1.93 (m, 1H), 1.86-1.64 (m, 3H); ¹³C NMR (100.6 MHz, CDCl₃): δ 164.8, 154.0, 131.9, 130.7, 129.8, 126.9, 125.9, 68.4, 63.8, 62.4, 57.7, 41.5, 28.9, 22.9.

### (1-methylpyrrolidin-2-yl)methyl 3,6-dichloro-2-methoxybenzoate hydrochloride 10·HCl

Prepared as per 2-(dimethylamino)ethyl 3,6-dichloro-2-methoxybenzoate hydrochloride except (1-methylpyrrolidin-2-yl)methyl 3,6-dichloro-2-methoxybenzoate was used. ¹H NMR (400 MHz, d₆-DMSO): δ 11.27 (br s, 1H), 7.70 (d, J=8.7 Hz, 1H), 7.43 (d, J=8.7 Hz, 1H), 4.81 (dd, J=12.2, 7.1 Hz, 1H), 4.71 (dd, J=12.3, 4.5 Hz, 1H), 3.87 (s, 3H), 3.75 (s, 1H), 3.58-3.51 (m, 1H), 3.12-3.05 (m, 1H), 2.83 (s, 3H), 2.34-2.21 (m, 1H), 2.06-1.74 (m, 3H); ¹³C NMR (100.6 MHz, d₆-DMSO): δ 163.3, 153.2, 133.0, 129.1, 128.6, 126.4, 126.2, 65.5, 63.9, 62.5, 56.2, 27.0, 21.4.

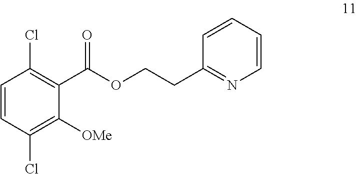 [SMILES: COc1c(Cl)ccc(Cl)c1C(=O)OCCc1ccccn1]

### 2-(pyridin-2-yl)ethyl 3,6-dichloro-2-methoxybenzoate 11

Prepared as per 2-(dimethylamino)ethyl 3,6-dichloro-2-methoxybenzoate except 2-pyridineethanol was used. ¹H NMR (400 MHz, CDCl₃): δ 8.59-8.52 (m, 1H), 7.61 (td, J=7.7, 1.9 Hz, 1H), 7.32 (d, J=8.7 Hz, 1H), 7.27-7.20 (m, 1H), 7.19-7.12 (m, 1H), 7.08 (d, J=8.6 Hz, 1H), 4.80 (t, J=6.8 Hz, 2H), 3.80 (s, 3H), 3.26 (t, J=6.8 Hz, 2H); ¹³C NMR (100.6 MHz, CDCl₃): δ 164.6, 157.7, 154.0, 149.6, 136.6, 131.9, 130.6, 129.8, 126.8, 125.9, 123.7, 121.9, 65.4, 62.3, 37.3.

### 2-(pyridin-2-yl)ethyl 3,6-dichloro-2-methoxybenzoate hydrochloride 11·HCl

Prepared as per 2-(dimethylamino)ethyl 3,6-dichloro-2-methoxybenzoate hydrochloride except 2-(pyridin-2-yl)ethyl 3,6-dichloro-2-methoxybenzoate was used. 1H NMR (600 MHz, d₆-DMSO): δ 8.80-8.77 (m, 1H), 8.41-8.36 (m, 1H), 7.96-7.89 (m, 1H), 7.84-7.77 (m, 1H), 7.64 (d, J=8.7 Hz, 1H), 7.34 (d, J=8.7 Hz, 1H), 4.84-4.78 (m, 2H), 3.70 (s, 3H), 3.5-3.4 (m, 2H); ¹³C NMR (150.9 MHz, d₆-DMSO): δ 163.6, 154.0, 153.1, 144.2, 143.3, 132.7, 129.4, 128.5, 126.8, 126.4, 126.1, 124.7, 64.2, 62.2, 32.9.

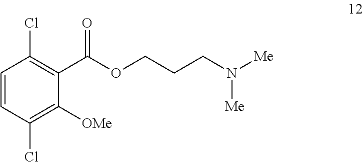 [SMILES: COc1c(Cl)ccc(Cl)c1C(=O)OCCCN(C)C]

### 3-(dimethylamino)propyl 3,6-dichloro-2-methoxybenzoate 12

Prepared as per 2-(dimethylamino)ethyl 3,6-dichloro-2-methoxybenzoate except 3-dimethylamino-1-propanol was used. ¹H NMR (400 MHz, CDCl₃): δ 7.31 (d, J=8.7 Hz, 1H), 7.08 (d, J=8.7 Hz, 1H), 4.41 (t, J=6.5 Hz, 2H), 3.88 (s, 3H), 2.38 (d, J=6.9 Hz, 2H), 2.20 (s, 6H), 1.96-1.84 (m, 2H); ¹³C NMR (100.6 MHz, CDCl₃): δ 164.6, 153.8, 131.8, 130.7, 129.6, 126.8, 125.9, 64.7, 62.3, 56.1, 45.6, 27.0.

### 3-(dimethylamino)propyl 3,6-dichloro-2-methoxybenzoate hydrochloride 12·HCl

Prepared as per 2-(dimethylamino)ethyl 3,6-dichloro-2-methoxybenzoate hydrochloride except 3-(dimethylamino)propyl 3,6-dichloro-2-methoxybenzoate was used. ¹H NMR (500 MHz, d₆-DMSO): δ 10.94 (br s, 1H), 7.68 (d, J=8.7 Hz, 1H), 7.41 (d, J=8.7 Hz, 1H), 4.43 (t, J=6.3 Hz, 2H), 3.85 (s, 3H), 3.20-3.09 (m, 2H), 2.73 (s, 6H), 2.20-2.11 (m, 2H); ¹³C NMR (125.8 MHz, d₆-DMSO): δ 163.7, 153.1, 132.7, 129.6, 128.6, 126.4, 126.1, 63.4, 62.3, 53.5, 42.0, 23.2.

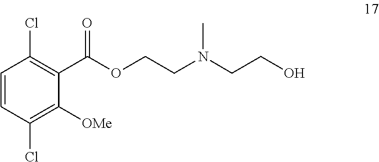 [SMILES: COc1c(Cl)ccc(Cl)c1C(=O)OCCN(C)CCO]

### 2-((2-hydroxyethyl)(methyl)amino)ethyl 3,6-dichloro-2-methoxybenzoate 17

Prepared as per 2-(dimethylamino)ethyl 3,6-dichloro-2-methoxybenzoate except N-methyldiethanolamine was used. ¹H NMR (400 MHz, CDCl₃): δ 7.34 (d, J=8.6 Hz, 1H), 7.10 (d, J=8.6 Hz, 1H), 4.47 (t, J=5.7 Hz, 2H), 3.90 (s, 3H), 3.57 (t, J=5.3 Hz, 2H), 2.82 (t, J=5.8 Hz, 2H), 2.78 (br s, 1H), 2.61 (t, J=5.3 Hz, 2H), 2.34 (s, 3H); ¹³C NMR (100.6 MHz, CDCl₃): δ 164.7, 153.9, 131.9, 130.5, 129.6, 126.9, 126.0, 63.6, 62.4, 59.1, 58.5, 55.9, 41.9.

### 2-((2-hydroxyethyl)(methyl)amino)ethyl 3,6-dichloro-2-methoxybenzoate hydrochloride 17·HCl

Prepared as per 2-(dimethylamino)ethyl 3,6-dichloro-2-methoxybenzoate hydrochloride except 2-((2-hydroxyethyl)(methyl)amino)ethyl 3,6-dichloro-2-methoxybenzoate was used. ¹H NMR (400 MHz, d₆-DMSO): δ 10.75 (s, 1H), 7.69 (d, J=8.7 Hz, 1H), 7.42 (d, J=8.7 Hz, 1H), 5.39 (br s, 1H), 4.77 (t, J=5.2 Hz, 2H), 3.86 (s, 3H), 3.77 (t, J=5.3 Hz, 2H), 3.60-3.56 (m, 2H), 3.25-3.21 (m, 2H), 2.83 (s, 3H); ¹³C NMR (100.6 MHz, d₆-DMSO): δ 163.4, 153.2, 132.9, 129.1, 128.6, 126.4, 126.1, 62.4, 60.7, 57.5, 55.3, 53.4, 40.4.

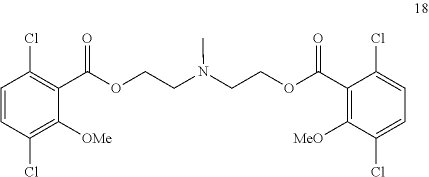 [SMILES: COc1c(Cl)ccc(Cl)c1C(=O)OCCN(C)CCOC(=O)c1c(Cl)ccc(Cl)c1OC]

### (Methylazanediyl)bis(ethane-2,1-diyl) bis(3,6-dichloro-2-methoxybenzoate)18

Prepared as per 2-(dimethylamino)ethyl 3,6-dichloro-2-methoxybenzoate except N-methyldiethanolamine (1 equivalent) and pyridine (1.5 equivalents) were used. ¹H NMR (400 MHz, CDCl₃): δ 7.34 (d, J=8.7 Hz, 2H), 7.09 (d, J=8.6 Hz, 2H), 4.47 (t, J=6.0 Hz, 4H), 3.89 (s, 6H), 2.87 (t, J=6.0 Hz, 4H), 2.41 (s, 3H); ¹³C NMR (100.6 MHz, CDCl₃): δ 164.6, 153.9, 131.9, 130.5, 129.7, 126.8, 125.9, 63.9, 62.4, 55.8, 42.6.

### (Methylazanediyl)bis(ethane-2,1-diyl) bis(3,6-dichloro-2-methoxybenzoate) hydrochloride 18·HCl

Prepared as per 2-(dimethylamino)ethyl 3,6-dichloro-2-methoxybenzoate hydrochloride except (methylazanediyl)bis(ethane-2,1-diyl) bis(3,6-dichloro-2-methoxybenzoate) was used. ¹H NMR (400 MHz, d₆-DMSO): δ 11.48 (br s, 1H), 7.68 (d, J=8.7 Hz, 2H), 7.40 (d, J=8.8 Hz, 2H), 4.79-4.72 (m, 4H), 3.83 (s, 6H), 3.59-3.55 (m, 4H), 2.84 (s, 3H); ¹³C NMR (100.6 MHz, d₆-DMSO): δ 163.3, 153.2, 132.9, 129.1, 128.6, 126.4, 126.1, 62.4, 60.7, 53.5, 40.4.

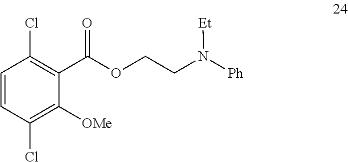 [SMILES: CCN(CCOC(=O)c1c(Cl)ccc(Cl)c1OC)c1ccccc1]

### 2-(ethyl(phenyl)amino)ethyl 3,6-dichloro-2-methoxybenzoate 24

Prepared as per 2-(dimethylamino)ethyl 3,6-dichloro-2-methoxybenzoate except 2-(N-ethylanilino)ethanol was used. ¹H NMR (400 MHz, CDCl₃): δ 7.37 (d, J=8.7 Hz, 1H), 7.28-7.19 (m, 2H), 7.13 (d, J=8.7 Hz, 1H), 6.80-6.67 (m, 3H), 4.52 (t, J=6.7 Hz, 2H), 3.90 (s, 3H), 3.69 (t, J=6.7 Hz, 2H), 3.45 (q, J=7.0 Hz, 2H), 1.17 (t, J=7.0 Hz, 3H); ¹³C NMR (100.6 MHz, CDCl₃): δ 164.7, 154.0, 147.5, 132.0, 130.5, 129.7, 129.6, 126.9, 126.0, 116.6, 112.2, 63.4, 62.4, 48.8, 45.6, 12.4.

### 2-(ethyl(phenyl)amino)ethyl 3,6-dichloro-2-methoxybenzoate hydrochloride 24·HCl

Prepared as per 2-(dimethylamino)ethyl 3,6-dichloro-2-methoxybenzoate hydrochloride except 2-(ethyl(phenyl)amino)ethyl 3,6-dichloro-2-methoxybenzoate was used. ¹H NMR (500 MHz, d₆-DMSO): δ 7.67 (d, J=8.7 Hz, 1H), 7.39 (d, J=8.7 Hz, 1H), 7.27-7.11 (m, 3H), 6.88 (s, 2H), 4.46 (t, J=6.1 Hz, 2H), 3.80 (s, 3H), 3.73-3.67 (m, 2H), 3.46-3.39 (m, 2H), 1.05 (t, J=7.0 Hz, 3H); ¹³C NMR (125.8 MHz, d₆-DMSO): δ 153.1, 132.7, 129.6, 129.4, 128.9, 128.6, 128.2, 126.4, 126.1, 125.3, 113.2, 63.1, 62.2, 49.0, 45.6, 11.6.

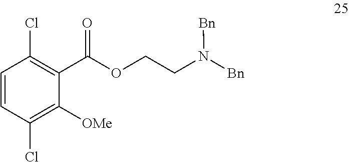 [SMILES: COc1c(Cl)ccc(Cl)c1C(=O)OCCN(Cc1ccccc1)Cc1ccccc1]

### 2-(dibenzylamino)ethyl 3,6-dichloro-2-methoxybenzoate 25

Prepared as per 2-(dimethylamino)ethyl 3,6-dichloro-2-methoxybenzoate except N,N-dibenzylethanolamine was used. ¹H NMR (400 MHz, CDCl₃): δ 7.40-7.18 (m, 11H), 7.11 (d, J=8.7 Hz, 1H), 4.45 (t, J=6.2 Hz, 2H), 3.84 (s, 3H), 3.69 (s, 4H), 2.87 (t, J=6.1 Hz, 2H); ¹³C NMR (100.6 MHz, CDCl₃): δ 164.7, 154.0, 139.3, 131.9, 130.7, 129.7, 128.9, 128.4, 127.2, 126.8, 125.9, 64.4, 62.4, 58.8, 51.6.

### 2-(dibenzylamino)ethyl 3,6-dichloro-2-methoxybenzoate hydrochloride 25·HCl

Prepared as per 2-(dimethylamino)ethyl 3,6-dichloro-2-methoxybenzoate hydrochloride except 2-(dibenzylamino)ethyl 3,6-dichloro-2-methoxybenzoate was used. ¹H NMR (500 MHz, d₆-DMSO): δ 11.57 (br s, 1H), 7.68 (d, J=8.7 Hz, 1H), 7.62 (s, 4H), 7.45-7.41 (m, 6H), 7.40 (d, J=8.7 Hz, 1H), 4.83-4.70 (m, 2H), 4.45-4.28 (m, 4H), 3.76 (s, 3H), 3.4 (m, 2H); ¹³C NMR (125.8 MHz, d₆-DMSO): δ 163.1, 153.0, 132.8, 131.4, 131.3, 129.5, 128.75, 128.71, 128.5, 126.3, 126.0, 62.2, 60.2, 56.7, 49.5.

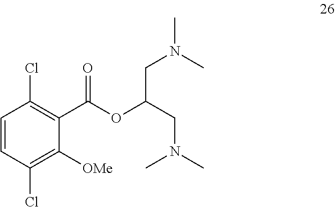 [SMILES: COc1c(Cl)ccc(Cl)c1C(=O)OC(CN(C)C)CN(C)C]

### 1,3-bis(dimethylamino)propan-2-yl 3,6-dichloro-2-methoxybenzoate 26

Prepared as per 2-(dimethylamino)ethyl 3,6-dichloro-2-methoxybenzoate except 1,3-bis(dimethylamino)2-propanol was used. ¹H NMR (400 MHz, CDCl₃): δ 7.33 (d, J=8.7 Hz, 1H), 7.09 (d, J=8.6 Hz, 1H), 5.37 (p, J=5.9 Hz, 1H), 3.92 (s, 3H), 2.65-2.55 (m, 4H), 2.30 (s, 12H); ¹³C NMR (100.6 MHz, CDCl₃): δ 164.2, 154.0, 131.7, 130.8, 129.7, 126.9, 126.0, 72.8, 62.4, 61.0, 46.2.

### 1,3-bis(dimethylamino)propan-2-yl 3,6-dichloro-2-methoxybenzoate dihydrochloride 26·HCl

Prepared as per 2-(dimethylamino)ethyl 3,6-dichloro-2-methoxybenzoate hydrochloride except 1,3-bis(dimethylamino)propan-2-yl 3,6-dichloro-2-methoxybenzoate was used. ¹H NMR (400 MHz, d₆-DMSO): δ 11.43 (s, 2H), 7.73 (d, J=8.7 Hz, 1H), 7.45 (d, J=8.7 Hz, 1H), 6.02-5.95 (m, 1H), 4.09-3.99 (m, 2H), 3.89 (s, 3H), 3.49 (dd, J=13.4, 5.2 Hz, 2H), 2.88 (s, 12H); ¹³C NMR (100.6 MHz, d₆-DMSO): δ 162.8, 153.3, 133.4, 128.5, 128.3, 126.6, 126.1, 79.3, 66.9, 62.6, 56.2, 43.7, 42.1.

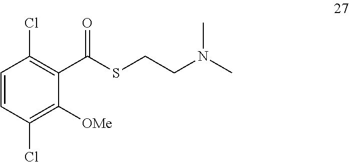 [SMILES: COc1c(Cl)ccc(Cl)c1C(=O)SCCN(C)C]

### S-(2-(dimethylamino)ethyl) 3,6-dichloro-2-methoxybenzene-1-carbothioate 27

Prepared as per 2-(dimethylamino)ethyl 3,6-dichloro-2-methoxybenzoate except 2-(dimethylamino)ethanethiol hydrochloride and pyridine (2 equivalents) were used. ¹H NMR (400 MHz, CDCl₃): δ 7.35 (d, J=8.6 Hz, 1H), 7.11 (d, J=8.7 Hz, 1H), 3.90 (s, 3H), 3.24 (d, J=6.9 Hz, 2H), 2.62 (d, J=7.3 Hz, 2H), 2.31 (s, 6H); ¹³C NMR (100.6 MHz, CDCl₃): δ 191.0, 153.6, 135.3, 132.2, 129.3, 127.0, 126.2, 62.9, 58.3, 45.4, 28.1

### S-(2-(dimethylamino)ethyl) 3,6-dichloro-2-methoxybenzene-1-carbothioate hydrochloride 27·HCl

Prepared as per 2-(dimethylamino)ethyl 3,6-dichloro-2-methoxybenzoate hydrochloride except S-(2-(dimethylamino)ethyl) 3,6-dichloro-2-methoxybenzene-1-carbothioate was used. ¹H NMR (400 MHz, d₆-DMSO): δ 11.23 (br s, 1H), 7.71 (d, J=8.8 Hz, 1H), 7.42 (d, J=8.7 Hz, 1H), 3.85 (s, 3H), 3.56-3.47 (m, 2H), 3.35-3.26 (m, 2H), 2.82 (s, 6H); ¹³C NMR (100.6 MHz, d₆-DMSO): δ 189.4, 152.8, 133.7, 133.3, 128.1, 126.7, 126.3, 62.8, 54.6, 41.9, 23.5.

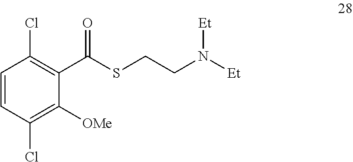 [SMILES: CCN(CC)CCSC(=O)c1c(Cl)ccc(Cl)c1OC]

### S-(2-(diethylamino)ethyl) 3,6-dichloro-2-methoxybenzene-1-carbothioate 28

Prepared as per 2-(dimethylamino)ethyl 3,6-dichloro-2-methoxybenzoate except 2-diethylaminoethanethiol hydrochloride and pyridine (2 equivalents) were used. ¹H NMR (400 MHz, CDCl₃): δ 7.34 (d, J=8.6 Hz, 1H), 7.10 (d, J=8.7 Hz, 1H), 3.90 (s, 3H), 3.20 (d, J=7.1 Hz, 2H), 2.77 (d, J=7.4 Hz, 2H), 2.62 (q, J=7.1 Hz, 4H), 1.07 (t, J=7.2 Hz, 6H); ¹³C NMR (100.6 MHz, CDCl₃): δ 191.1, 153.6, 135.4, 132.1, 129.3, 126.9, 126.2, 62.8, 51.9, 47.2, 27.9, 12.2.

### S-(2-(diethylamino)ethyl) 3,6-dichloro-2-methoxybenzene-1-carbothioate hydrochloride 28·HCl

Prepared as per 2-(dimethylamino)ethyl 3,6-dichloro-2-methoxybenzoate hydrochloride except S-(2-(diethylamino)ethyl) 3,6-dichloro-2-methoxybenzene-1-carbothioate was used. ¹H NMR (400 MHz, d₆-DMSO): δ 10.88 (br s, 1H), 7.71 (d, J=8.7 Hz, 1H), 7.43 (d, J=8.7 Hz, 1H), 3.85 (s, 3H), 3.54-3.46 (m, 2H), 3.30-3.14 (m, 6H), 1.28 (t, J=7.2 Hz, 6H); ¹³C NMR (100.6 MHz, d₆-DMSO): δ190.0, 152.8, 133.8, 133.4, 128.1, 126.7, 126.3, 62.8, 48.8, 46.6, 23.3, 8.6.

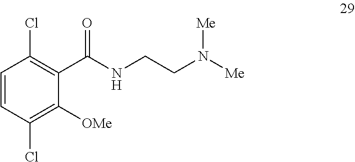 [SMILES: COc1c(Cl)ccc(Cl)c1C(=O)NCCN(C)C]

### 3,6-dichloro-N-(2-(dimethylamino)ethyl)-2-methoxybenzamide 29

Prepared as per 2-(dimethylamino)ethyl 3,6-dichloro-2-methoxybenzoate except N,N-dimethylethylenediamine and triethylamine (1.1 equivalents) were used. ¹H NMR (600 MHz, d₆-DMSO): δ 8.54 (t, J=5.6 Hz, 1H), 7.53 (d, J=8.7 Hz, 1H), 7.29 (d, J=8.7 Hz, 1H), 3.81 (s, 3H), 3.33-3.30 (m, 2H), 2.38 (t, J=6.9 Hz, 2H), (s, 6H); ¹³C NMR (150.9 MHz, d₆-DMSO): δ 163.0, 153.0, 134.0, 130.9, 129.2, 125.9, 125.8, 61.9, 57.9, 45.2, 37.3.

### 3,6-dichloro-N-(2-(dimethylamino)ethyl)-2-methoxybenzamide hydrochloride 29·HCl

Prepared as per 2-(dimethylamino)ethyl 3,6-dichloro-2-methoxybenzoate hydrochloride except 3,6-dichloro-N-(2-(dimethylamino)ethyl)-2-methoxybenzamide was used. ¹H NMR (500 MHz, d₆-DMSO): δ 10.99 (br s, 1H), 9.01 (t, J=5.6 Hz, 1H), 7.58 (d, J=8.7 Hz, 1H), 7.33 (d, J=8.7 Hz, 1H), 3.82 (s, 3H), 3.69-3.64 (m, 2H), 3.21 (t, J=6.8 Hz, 2H), 2.80 (s, 6H); ¹³C NMR (125.8 MHz, d₆-DMSO): δ 163.5, 153.1, 133.3, 131.4, 129.1, 126.1, 125.9, 62.1, 54.9, 42.2, 34.3.

FIG. 2 provides a scheme for the synthesis of 2-(2-(dimethylamino)ethoxy)ethyl 3,6-dichloro-2-methoxybenzoate hydrochloride 16·HCl from the readily available commercial pesticide dicamba.

### 2-(2-bromoethoxy)ethyl 3,6-dichloro-2-methoxybenzoate

Prepared as per 4-bromobutyl 3,6-dichloro-2-methoxybenzoate except 2-(2-bromoethoxy)ethanol was used. ¹H NMR (400 MHz, CDCl₃): δ 7.35 (d, J=8.7 Hz, 1H), 7.12 (d, J=8.7 Hz, 1H), 4.54 (d, J=4.8 Hz, 2H), 3.92 (s, 3H), 3.88-3.79 (m, 4H), 3.46 (d, J=6.2 Hz, 2H); ¹³C NMR (100.6 MHz, CDCl₃): δ 164.6, 154.0, 132.0, 130.3, 129.8, 126.9, 126.0, 71.3, 68.9, 65.1, 62.5, 30.2.

### 2-(2-(dimethylamino)ethoxy)ethyl 3,6-dichloro-2-methoxybenzoate 16

A solution of 2-(2-(dimethylamino)ethoxy)ethyl 3,6-dichloro-2-methoxybenzoate hydrochloride (200 mg, 0.54 mmol) in CH₂Cl₂ (10 ml) was washed with sat. NaHCO₃ (15 ml), brine (15 ml), dried (MgSO₄), filtered and concentrated to obtain 2-(2-(dimethylamino)ethoxy)ethyl 3,6-dichloro-2-methoxybenzoate as a colourless oil (181 mg). ¹H NMR (400 MHz, CDCl₃): δ 7.35 (d, J=8.6 Hz, 1H), 7.11 (d, J=8.7 Hz, 1H), 4.57-4.50 (m, 2H), 3.91 (s, 3H), 3.82-3.75 (m, 2H), 3.60 (t, J=5.8 Hz, 2H), 2.50 (t, J=5.7 Hz, 2H), 2.25 (s, 6H); ¹³C NMR (100.6 MHz, CDCl₃): δ 164.5, 153.9, 131.8, 130.3, 129.7, 126.7, 125.8, 69.4, 68.7, 65.1, 62.3, 58.8, 45.9.

### 2-(2-(dimethylamino)ethoxy)ethyl 3,6-dichloro-2-methoxybenzoate hydrochloride 16·HCl

A solution of 2-(2-bromoethoxy)ethyl 3,6-dichloro-2-methoxybenzoate (505 mg, 1.36 mmol) and dimethylamine (2 M in THF, 6.8 ml, 13.6 mmol) in THF (3 ml) was stirred at reflux for 24 h. The mixture was then transferred to a separating funnel and washed with sat. NaHCO₃ (15 ml), brine (15 ml), dried (MgSO₄), filtered, acidified with saturated hydrogen chloride in ether until the resultant solution was pH=˜2 (˜1 ml) and concentrated. The resultant residue was then purified by flash column chromatography (MeOH:CHCl₃ 1:9) to obtain 2-(2-(dimethylamino)ethoxy)ethyl 3,6-dichloro-2-methoxybenzoate hydrochloride as a white solid (415 mg, 82%). ¹H NMR (400 MHz, d₆-DMSO): δ 7.68 (d, J=8.7 Hz, 1H), 7.40 (d, J=8.8 Hz, 1H), 4.52-4.46 (m, 2H), 3.85 (s, 3H), 3.81-3.73 (m, 4H), 3.15 (t, J=5.2 Hz, 2H), 2.67 (s, 6H); ¹³C NMR (100.6 MHz, d₆-DMSO): δ 163.7, 153.1, 132.6, 129.7, 128.5, 126.3, 126.1, 68.0, 65.1, 65.0, 62.3, 55.8, 42.9.

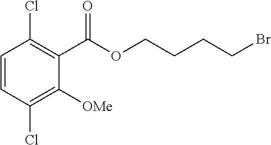 [SMILES: COc1c(Cl)ccc(Cl)c1C(=O)OCCCCBr]

### 4-bromobutyl 3,6-dichloro-2-methoxybenzoate

To a solution of 4-bromo-1-butanol (0.45 ml, 3.3 mmol) and pyridine (0.35 ml, 4.4 mmol) in CH₂Cl₂ (7.5 ml) was added 3,6-dichloro-2-methoxybenzoyl chloride (530 mg, 2.2 mmol) in CH₂Cl₂ (7.5 ml) at 0° C. and the resultant solution stirred at room temperature for 24 h. The mixture was then transferred to a separating funnel and washed with sat. NaHCO₃ (15 ml), brine (15 ml), dried (MgSO₄), filtered and concentrated. The resultant residue was then purified by flash column chromatography (EtOAc:hexanes 5:95) to obtain 4-bromobutyl 3,6-dichloro-2-methoxybenzoate as a colourless oil (498 mg, 62%, over two steps). ¹H NMR (400 MHz, CDCl₃): δ 7.36 (d, J=8.6 Hz, 1H), 7.12 (d, J=8.7 Hz, 1H), 4.42 (t, J=6.1 Hz, 2H), 3.91 (s, 3H), 3.46 (t, J=6.4 Hz, 2H), 2.09-1.88 (m, 4H); 130 NMR (100.6 MHz, CDCl₃): δ 164.7, 153.9, 132.0, 130.6, 129.7, 126.9, 126.0, 65.2, 62.4, 33.1, 29.2, 27.4.

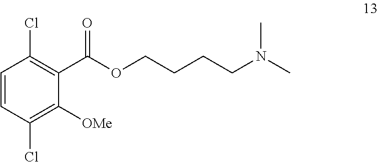 [SMILES: COc1c(Cl)ccc(Cl)c1C(=O)OCCCCN(C)C]

### 4-(dimethylamino)butyl 3,6-dichloro-2-methoxybenzoate hydrochloride 13·HCl

Prepared as per 2-(2-(dimethylamino)ethoxy)ethyl 3,6-dichloro-2-methoxybenzoate hydrochloride except 4-bromobutyl 3,6-dichloro-2-methoxybenzoate was used. ¹H NMR (400 MHz, d₆-DMSO): δ 7.67 (d, J=8.7 Hz, 1H), 7.40 (d, J=8.7 Hz, 1H), 4.36 (t, J=6.3 Hz, 2H), 3.83 (s, 3H), 2.59 (t, J=7.4 Hz, 2H), 2.38 (s, 6H), 1.78-1.66 (m, 2H), 1.66-1.55 (m, 2H); ¹³C NMR (100.6 MHz, d₆-DMSO): δ 164.3, 153.5, 133.0, 129.0, 128.0, 126.8, 125.9, 66.1, 62.7, 51.4, 43.7, 26.0, 21.8.

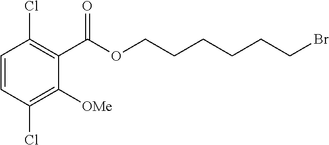 [SMILES: COc1c(Cl)ccc(Cl)c1C(=O)OCCCCCCBr]

### 6-bromohexyl 3,6-dichloro-2-methoxybenzoate

Prepared as per 4-bromobutyl 3,6-dichloro-2-methoxybenzoate except 6-bromo-1-hexanol was used. ¹H NMR (400 MHz, CDCl₃): δ 7.35 (d, J=8.7 Hz, 1H), 7.12 (d, J=8.7 Hz, 1H), 4.39 (t, J=6.6 Hz, 2H), 3.91 (s, 3H), 3.41 (t, J=6.8 Hz, 2H), 1.94-1.73 (m, 4H), 1.56-1.43 (m, 4H); ¹³C NMR (100.6 MHz, CDCl₃): δ 164.8, 153.9, 131.9, 130.8, 129.7, 126.9, 126.0, 66.2, 62.4, 33.8, 32.7, 28.6, 27.9, 25.2.

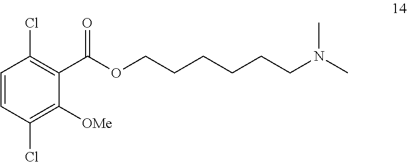 [SMILES: COc1c(Cl)ccc(Cl)c1C(=O)OCCCCCCN(C)C]

### 6-(dimethylamino)hexyl 3,6-dichloro-2-methoxybenzoate 14

Prepared as per 2-(2-(dimethylamino)ethoxy)ethyl 3,6-dichloro-2-methoxybenzoate except 6-(dimethylamino)hexyl 3,6-dichloro-2-methoxybenzoate hydrochloride was used. ¹H NMR (400 MHz, CDCl₃): δ 7.33 (d, J=8.7 Hz, 1H), 7.10 (d, J=8.7 Hz, 1H), 4.37 (t, J=6.6 Hz, 2H), 3.90 (s, 3H), 2.35-2.25 (m, 2H), 2.24 (d, J=1.0 Hz, 6H), 1.82-1.70 (m, 2H), 1.56-1.29 (m, 6H); ¹³C NMR (100.6 MHz, CDCl₃): δ 164.8, 153.9, 131.8, 130.8, 129.7, 126.8, 126.0, 66.3, 62.4, 59.7, 45.4, 28.6, 27.5, 27.1, 25.9.

### 6-(dimethylamino)hexyl 3,6-dichloro-2-methoxybenzoate hydrochloride 14·HCl

Prepared as per 2-(2-(dimethylamino)ethoxy)ethyl 3,6-dichloro-2-methoxybenzoate hydrochloride except 6-bromohexyl 3,6-dichloro-2-methoxybenzoate was used. ¹H NMR (400 MHz, d₆-DMSO): δ 10.81 (br s, 1H), 7.65 (d, J=8.7 Hz, 1H), 7.39 (d, J=8.7 Hz, 1H), 4.34 (t, J=6.5 Hz, 2H), 3.83 (s, 3H), 3.02-2.92 (m, 2H), 2.69 (d, J=4.8 Hz, 6H), 1.68 (ddt, J=15.0, 11.6, 7.2 Hz, 4H), 1.46-1.26 (m, 4H); ¹³C NMR (100.6 MHz, d₆-DMSO): δ 163.8, 153.0, 132.5, 130.0, 128.5, 126.4, 126.1, 65.9, 62.2, 56.2, 41.8, 27.8, 25.5, 24.8, 23.4.

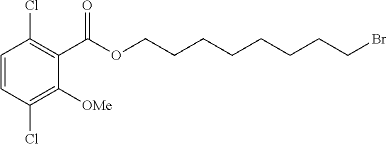 [SMILES: COc1c(Cl)ccc(Cl)c1C(=O)OCCCCCCCCBr]

### 8-bromooctyl 3,6-dichloro-2-methoxybenzoate

Prepared as per 4-bromobutyl 3,6-dichloro-2-methoxybenzoate except 8-bromo-1-octanol was used.

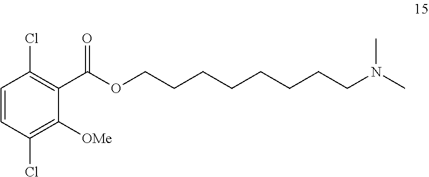 [SMILES: COc1c(Cl)ccc(Cl)c1C(=O)OCCCCCCCCN(C)C]

### 8-(dimethylamino)octyl 3,6-dichloro-2-methoxybenzoate 15

Prepared as per 2-(2-(dimethylamino)ethoxy)ethyl 3,6-dichloro-2-methoxybenzoate except 8-(dimethylamino)octyl 3,6-dichloro-2-methoxybenzoate hydrochloride was used. ¹H NMR (400 MHz, CDCl₃): δ 7.33 (d, J=8.7 Hz, 1H), 7.10 (d, J=8.6 Hz, 1H), 4.36 (t, J=6.7 Hz, 2H), 3.89 (s, 3H), 2.36-2.26 (m, 2H), 2.26 (s, 6H), 1.80-1.68 (m, 2H), 1.54-1.36 (m, 4H), 1.36-1.20 (m, 6H); ¹³C NMR (100.6 MHz, CDCl₃): δ 164.8, 153.9, 131.8, 130.9, 129.7, 126.8, 125.9, 66.4, 62.3, 59.8, 45.3, 29.5, 29.2, 28.6, 27.4, 27.4, 25.9.

### 8-(dimethylamino)octyl 3,6-dichloro-2-methoxybenzoate hydrochloride 15·HCl

Prepared as per 2-(2-(dimethylamino)ethoxy)ethyl 3,6-dichloro-2-methoxybenzoate hydrochloride except 8-bromooctyl 3,6-dichloro-2-methoxybenzoate was used. ¹H NMR (400 MHz, d₆-DMSO): δ 7.66 (d, J=8.7 Hz, 1H), 7.39 (d, J=8.7 Hz, 1H), 4.33 (t, J=6.5 Hz, 2H), 3.82 (s, 3H), 3.00-2.91 (m, 2H), 2.68 (s, 6H), 1.75-1.57 (m, 4H), 1.46-1.17 (m, 8H); ¹³C NMR (100.6 MHz, d₆-DMSO): δ 163.8, 153.0, 132.5, 130.1, 128.4, 126.4, 126.1, 66.0, 62.2, 56.4, 41.9, 28.4, 28.3, 28.0, 25.9, 25.2, 23.6.

FIG. 3 provides a scheme for the synthesis of 2-aminoethyl 3,6-dichloro-2-methoxybenzoate hydrochloride 19·HCl from the readily available commercial pesticide dicamba.

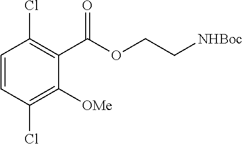 [SMILES: COc1c(Cl)ccc(Cl)c1C(=O)OCCNC(=O)OC(C)(C)C]

### 2-(tert-butoxycarbonylamino)ethyl 3,6-dichloro-2-methoxybenzoate

Prepared as per 4-bromobutyl 3,6-dichloro-2-methoxybenzoate except 2-(tert-butoxycarbonylamino)-1-ethanol was used. ¹H NMR (400 MHz, CDCl₃): δ 7.39 (d, J=8.6 Hz, 1H), 7.15 (d, J=8.6 Hz, 1H), 4.48 (t, J=5.2 Hz, 2H), 3.94 (s, 3H), 3.56-3.50 (m, 2H), 1.47 (s, 9H); ¹³C NMR (100.6 MHz, CDCl₃): δ 164.4, 153.8, 153.0, 132.0, 130.8, 129.5, 126.8, 125.9, 65.2, 62.4, 43.0, 28.4, 13.8.

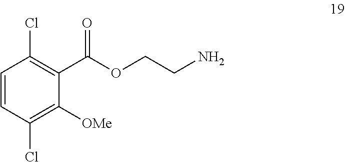 [SMILES: COc1c(Cl)ccc(Cl)c1C(=O)OCCN]

### 2-aminoethyl 3,6-dichloro-2-methoxybenzoate 19

Trifluoroacetic acid (1 ml) was added to a solution of 2-(tert-butoxycarbonylamino)ethyl 3,6-dichloro-2-methoxybenzoate (250 mg, 0.69 mmol) in CH₂Cl₂ (4 ml) and the resultant solution was stirred at room temperature for 2 h. The solution was then diluted with CH₂Cl₂ and transferred to a separating funnel, washed with sat. NaHCO₃ (15 ml), brine (15 ml), dried (MgSO₄), filtered and concentrated to give 2-aminoethyl 3,6-dichloro-2-methoxybenzoate as a white solid (175 mg, 96%). ¹H NMR (400 MHz, CDCl₃): δ 7.36 (d, J=8.6 Hz, 1H), 7.12 (d, J=8.7 Hz, 1H), 4.42 (t, J=5.3 Hz, 2H), 3.92 (s, 3H), 3.06 (t, J=5.3 Hz, 2H); ¹³C NMR (100.6 MHz, CDCl₃): δ 164.7, 153.9, 132.0, 130.6, 129.6, 126.9, 126.0, 68.6, 62.5, 41.1.

### 2-aminoethyl 3,6-dichloro-2-methoxybenzoate hydrochloride 19·HCl

Prepared as per 2-(dimethylamino)ethyl 3,6-dichloro-2-methoxybenzoate hydrochloride except 2-aminoethyl 3,6-dichloro-2-methoxybenzoate was used. ¹H NMR (400 MHz, d₆-DMSO): δ 8.11 (br s, 3H), 7.70 (d, J=8.7 Hz, 1H), 7.42 (d, J=8.7 Hz, 1H), 4.52 (t, J=5.5 Hz, 2H), 3.87 (s, 3H), 3.19 (t, J=5.6 Hz, 2H); ¹³C NMR (100.6 MHz, d₆-DMSO): a 164.0, 153.7, 133.3, 129.7, 129.3, 126.8, 126.5, 63.3, 62.8, 38.1.

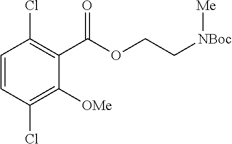 [SMILES: COc1c(Cl)ccc(Cl)c1C(=O)OCCN(C)C(=O)OC(C)(C)C]

### 2-(N-(tert-butyloxycarbonyl)-N-methyl-amino)ethyl 3,6-dichloro-2-methoxybenzoate

Prepared as per 4-bromobutyl 3,6-dichloro-2-methoxybenzoate except tert-butyl N-(2-hydroxyethyl)-N-methylcarbamate was used. ¹H NMR (400 MHz, CDCl₃): δ 7.35 (d, J=8.6 Hz, 1H), 7.11 (d, J=8.6 Hz, 1H), 4.51-4.44 (m, 2H), 3.90 (s, 3H), 3.60-3.53 (m, 2H), 2.93 (s, 3H), 1.44 (s, 9H); ¹³C NMR (100.6 MHz, CDCl₃): δ ¹³C NMR (101 MHz, CDCl₃) δ 164.6, 155.5, 154.0, 132.0, 130.5, 129.7, 126.9, 126.0, 80.0, 64.2, 62.4, 47.7, 35.7, 28.5.

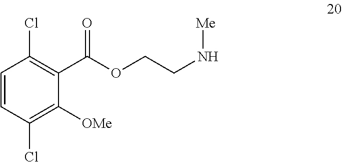 [SMILES: CNCCOC(=O)c1c(Cl)ccc(Cl)c1OC]

### 2-(methylamino)ethyl 3,6-dichloro-2-methoxybenzoate 20

Prepared as per 2-aminoethyl 3,6-dichloro-2-methoxybenzoate except 2-(N-(tert-butyloxycarbonyl)-N-methyl-amino)ethyl 3,6-dichloro-2-methoxybenzoate was used. 1H NMR (400 MHz, CDCl₃): δ 7.34 (d, J=8.7 Hz, 1H), 7.10 (d, J=8.7 Hz, 1H), 4.49 (t, J=5.3 Hz, 2H), 3.90 (s, 3H), 2.98-2.91 (m, 2H), 2.47 (s, 3H); ¹³C NMR (100.6 MHz, CDCl₃): δ 164.6, 153.9, 131.9, 130.5, 129.6, 126.9, 126.0, 65.3, 62.4, 50.1, 36.1.

### 2-(methylamino)ethyl 3,6-dichloro-2-methoxybenzoate hydrochloride 20·HCl

Prepared as per 2-(dimethylamino)ethyl 3,6-dichloro-2-methoxybenzoate hydrochloride except 2-(methylamino)ethyl 3,6-dichloro-2-methoxybenzoate was used. ¹H NMR (400 MHz, d₆-DMSO): δ 9.13 (br s, 2H), 7.70 (d, J=8.7 Hz, 1H), 7.42 (d, J=8.7 Hz, 1H), 4.64-4.57 (m, 2H), 3.87 (s, 3H), 3.31 (t, J=5.5 Hz, 2H), 2.57 (s, 3H); ¹³C NMR (100.6 MHz, d₆-DMSO): δ 163.4, 153.3, 132.9, 129.2, 128.8, 126.3, 126.1, 62.4, 61.6, 46.3, 32.7.

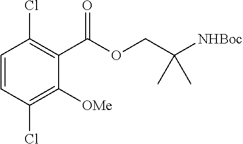 [SMILES: COc1c(Cl)ccc(Cl)c1C(=O)OCC(C)(C)NC(=O)OC(C)(C)C]

### 2-(tert-butoxycarbonylamino)-2-methylpropyl 3,6-dichloro-2-methoxybenzoate

Prepared as per 4-bromobutyl 3,6-dichloro-2-methoxybenzoate except N-Boc-2-amino-2-methyl-1-propanol was used. ¹H NMR (400 MHz, CDCl₃): δ 7.36 (d, J=8.7 Hz, 1H), 7.12 (d, J=8.7 Hz, 1H), 4.60 (br s, 1H), 4.45 (s, 2H), 3.90 (s, 3H), 1.42 (s, 9H), 1.36 (s, 6H); ¹³C NMR (100.6 MHz, CDCl₃): δ 164.5, 154.6 154.0, 132.0, 130.6, 129.7, 126.9, 126.0, 79.5, 70.4, 62.4, 52.2, 28.5, 24.6.

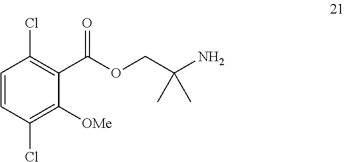 [SMILES: COc1c(Cl)ccc(Cl)c1C(=O)OCC(C)(C)N]

### 2-amino-2-methylpropyl 3,6-dichloro-2-methoxybenzoate 21

Prepared as per 2-aminoethyl 3,6-dichloro-2-methoxybenzoate except 2-(tert-butoxycarbonylamino)-2-methylpropyl 3,6-dichloro-2-methoxybenzoate was used. 1H NMR (400 MHz, CDCl₃): δ 7.36 (d, J=8.7 Hz, 1H), 7.12 (d, J=8.6 Hz, 1H), 4.16 (s, 2H), 3.91 (s, 3H), 1.19 (s, 6H); ¹³C NMR (100.6 MHz, CDCl₃): δ 164.7, 153.9, 132.0, 130.7, 129.6, 126.9, 126.0, 75.4, 62.4, 49.7, 27.3.

### 2-amino-2-methylpropyl 3,6-dichloro-2-methoxybenzoate hydrochloride 21·HCl

Prepared as per 2-(dimethylamino)ethyl 3,6-dichloro-2-methoxybenzoate hydrochloride except 2-amino-2-methylpropyl 3,6-dichloro-2-methoxybenzoate was used. ¹H NMR (400 MHz, d₆-DMSO): δ 7.53 (d, J=8.6 Hz, 1H), 7.28 (d, J=8.6 Hz, 1H), 4.05 (s, 2H), 3.83 (s, 3H), 1.08 (s, 6H); ¹³C NMR (100.6 MHz, d₆-DMSO): δ 163.6, 153.0, 131.9, 130.1, 128.5, 126.0, 125.9, 74.9, 61.8, 48.8, 26.8.

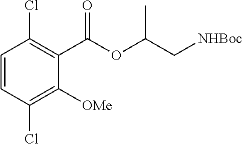 [SMILES: COc1c(Cl)ccc(Cl)c1C(=O)OC(C)CNC(=O)OC(C)(C)C]

### 1-(tert-butoxycarbonylamino)propan-2-yl 3,6-dichloro-2-methoxybenzoate

Prepared as per 4-bromobutyl 3,6-dichloro-2-methoxybenzoate except tert-butyl N-(2-hydroxypropyl)carbamate was used. δ ¹H NMR (400 MHz, CDCl₃): δ 7.37 (d, J=8.7 Hz, 1H), 7.36 (d, J=8.6 Hz, 1H), 7.13 (d, J=8.7 Hz, 1H), 7.12 (d, J=8.6 Hz, 1H), 5.50-5.22 (m, 1H), 5.02-4.86 (m, 1H), 4.69 (s, 1H), 4.45-4.30 (m, 2H), 4.11-3.99 (m, 1H), 3.93 (s, 3H), 3.91 (s, 3H), 3.60-3.45 (m, 1H), 3.28-3.14 (m, 1H), 1.45 (s, 9H), 1.44 (s, 9H), 1.36 (d, J=6.4 Hz, 3H), 1.24 (d, J=6.8 Hz, 3H); ¹³C NMR (100.6 MHz, CDCl₃): a ¹³C NMR (101 MHz, CDCl₃) δ 164.6, 164.1, 156.0, 155.2, 154.0, 153.8, 132.1, 132.0, 130.8, 130.5, 129.7, 129.5, 126.9, 126.9, 126.1, 126.0, 79.7, 72.7, 68.6, 62.5, 45.7, 45.1, 28.5, 18.0, 17.4.

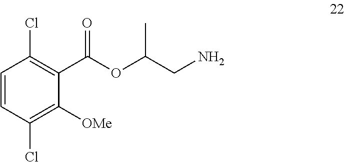 [SMILES: COc1c(Cl)ccc(Cl)c1C(=O)OC(C)CN]

### 1-aminopropan-2-yl 3,6-dichloro-2-methoxybenzoate 22

Prepared as per 2-aminoethyl 3,6-dichloro-2-methoxybenzoate except 1-(tert-butoxycarbonylamino)propan-2-yl 3,6-dichloro-2-methoxybenzoate was used. 1H NMR (400 MHz, CDCl₃): δ 7.34 (d, J=8.7 Hz, 1H), 7.11 (d, J=8.7 Hz, 1H), 5.27-5.14 (m, 1H), 3.91 (s, 3H), 2.98-2.89 (m, 2H), 1.36 (d, J=6.4 Hz, 3H); ¹³C NMR (100.6 MHz, CDCl₃): δ 164.4, 153.8, 131.0, 129.5, 126.9, 126.0, 75.4, 62.4, 62.4, 47.0, 17.4.

### 1-aminopropan-2-yl 3,6-dichloro-2-methoxybenzoate hydrochloride 22·HCl

Prepared as per 2-(dimethylamino)ethyl 3,6-dichloro-2-methoxybenzoate hydrochloride except 1-aminopropan-2-yl 3,6-dichloro-2-methoxybenzoate was used. ¹H NMR (400 MHz, d₆-DMSO): δ 8.33 (br s, 3H), 7.68 (d, J=8.7 Hz, 1H), 7.40 (d, J=8.7 Hz, 1H), 5.37-5.25 (m, 1H), 3.86 (s, 3H), 3.18-3.01 (m, 2H), 1.41 (d, J=6.3 Hz, 3H); ¹³C NMR (100.6 MHz, d₆-DMSO): δ 163.0, 153.2, 132.7, 129.4, 128.8, 126.3, 126.0, 70.1, 62.3, 42.1, 16.9.

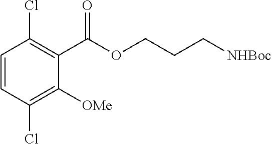 [SMILES: COc1c(Cl)ccc(Cl)c1C(=O)OCCCNC(=O)OC(C)(C)C]

### 3-(tert-butoxycarbonylamino)propyl 3,6-dichloro-2-methoxybenzoate

Prepared as per 4-bromobutyl 3,6-dichloro-2-methoxybenzoate except 3-(tert-butoxycarbonylamino)-1-propanol was used. ¹H NMR (400 MHz, CDCl₃): δ 7.35 (d, J=8.7 Hz, 1H), 7.11 (d, J=8.6 Hz, 1H), 4.81 (s, 1H), 4.44 (t, J=6.2 Hz, 2H), 3.90 (s, 3H), 3.32-3.22 (m, 2H), 2.00-1.90 (m, 2H), 1.43 (s, 9H); ¹³C NMR (100.6 MHz, CDCl₃): δ 164.8, 156.1, 153.9, 132.0, 130.6, 129.6, 126.9, 126.0, 79.4, 63.9, 62.4, 37.5, 29.1, 28.5.

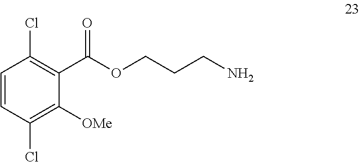 [SMILES: COc1c(Cl)ccc(Cl)c1C(=O)OCCCN]

### 3-aminopropyl 3,6-dichloro-2-methoxybenzoate 23

Prepared as per 2-aminoethyl 3,6-dichloro-2-methoxybenzoate except 3-(tert-butoxycarbonylamino)propyl 3,6-dichloro-2-methoxybenzoate was used. 1H NMR (400 MHz, CDCl₃): δ 7.34 (d, J=8.6 Hz, 1H), 7.11 (d, J=8.6 Hz, 1H), 4.47 (t, J=6.3 Hz, 2H), 3.90 (s, 3H), 2.90-2.85 (m, 2H), 1.96-1.85 (m, 2H); ¹³C NMR (100.6 MHz, CDCl₃): δ 164.8, 153.9, 131.9, 130.7, 129.7, 126.9, 126.0, 64.1, 62.4, 38.8, 32.6.

### 3-aminopropyl 3,6-dichloro-2-methoxybenzoate hydrochloride 23·HCl

Prepared as per 2-(dimethylamino)ethyl 3,6-dichloro-2-methoxybenzoate hydrochloride except 3-aminopropyl 3,6-dichloro-2-methoxybenzoate was used. 1H NMR (400 MHz, d₆-DMSO): δ 8.15 (br s, 3H), 7.67 (d, J=8.8 Hz, 1H), 7.40 (d, J=8.7 Hz, 1H), 4.44 (t, J=6.3 Hz, 2H), 3.84 (s, 3H), 2.94-2.85 (m, 2H), 2.10-1.98 (m, 2H); ¹³C NMR (100.6 MHz, d₆-DMSO): δ 163.7, 153.1, 132.7, 129.7, 128.5, 126.4, 126.2, 63.4, 62.3, 35.9, 26.4.

Different salts of 2-(dimethylamino)ethyl 3,6-dichloro-2-methoxybenzoate 1 are readily available.

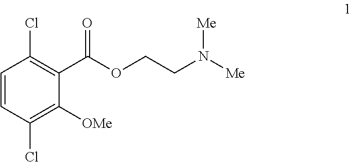 [SMILES: COc1c(Cl)ccc(Cl)c1C(=O)OCCN(C)C]

### 2-(dimethylamino)ethyl 3,6-dichloro-2-methoxybenzoate hydrobromide 1.HBr

Prepared as per 2-(dimethylamino)ethyl 3,6-dichloro-2-methoxybenzoate hydrochloride except hydrogen bromide in acetic acid was used. ¹H NMR (500 MHz, d₆-DMSO): δ 9.64 (br s, 1H), 7.71 (d, J=8.7 Hz, 1H), 7.43 (d, J=8.7 Hz, 1H), 4.68 (t, J=5.3 Hz, 2H), 3.86 (s, 3H), 3.52 (t, J=5.3 Hz, 2H), 2.83 (s, 6H); ¹³C NMR (125.8 MHz, d₆-DMSO): δ 163.3, 153.2, 133.0, 129.1, 128.7, 126.4, 126.1, 62.4, 60.6, 54.5, 42.8.

### 2-(dimethylammonium)ethyl 3,6-dichloro-2-methoxybenzoate bisulfate 1.H₂SO₄

2-(Dimethylamino)ethyl 3,6-dichloro-2-methoxybenzoate (50 mg, 0.17 mmol) was dissolved in diethyl ether (1 mL), and sulfuric acid (10 μL, 0.19 mmol) was added. The mixture was allowed to stand at r.t. for 1 hr, then the supernatant was decanted and the residue was washed with diethyl ether (2×1 mL), suspended in diethyl ether (2 mL) and concentrated in vacuo (×3) to yield a hygroscopic, colourless solid (62 mg, 93%). ¹H NMR (400 MHz, d₆-DMSO): 9.61 (bs, 1H), 7.71 (d, J=8.7 Hz, 1H), 7.43 (d, J=8.7 Hz, 1H), 4.69-4.67 (m, 2H), 3.86 (s, 3H), 3.55-3.51 (m, 2H), 2.84 (bs, 6H); ¹³C NMR (101 MHz, d₆-DMSO): 163.3, 153.2, 133.0, 129.1, 128.7, 126.4, 126.1, 62.4, 60.6, 54.5, 42.8.

### 2-(dimethylammonium)ethyl 3,6-dichloro-2-methoxybenzoate tosylate 1.TsOH

2-(Dimethylamino)ethyl 3,6-dichloro-2-methoxybenzoate (50 mg, 0.17 mmol) was dissolved in ethyl acetate (0.3 mL), and p-toluenesulfonic acid monohydrate in THF (1.0 M, 0.18 mL, 0.18 mmol) was added. The solution was concentrated in vacuo, co-evaporated with toluene (3×1 mL), and the residue washed with hexane (2×2 mL) and dried under reduced pressure to yield a colourless solid (80 mg, quant.). ¹H NMR (400 MHz, d₆-DMSO): 9.56 (bs, 1H), 7.71 (d, J=8.7 Hz, 1H), 7.48-7.46 (m, 2H), 7.43 (d, J=8.7 Hz, 1H), 7.12-7.10 (m, 2H), 4.69-4.66 (m, 2H), 3.86 (s, 3H), 3.55-3.51 (m, 2H), 2.84 (bs, 6H), 2.29 (s, 3H); ¹³C NMR (101 MHz, ds-DMSO): 163.3, 153.2, 145.7, 137.6, 132.9, 129.1, 128.7, 128.1, 126.4, 126.1, 125.5, 62.4, 60.5, 54.5, 42.7, 20.8.

### 2-(dimethylammonium)ethyl 3,6-dichloro-2-methoxybenzoate citrate 1.citrate

2-(Dimethylamino)ethyl 3,6-dichloro-2-methoxybenzoate (50 mg, 0.17 mmol) was dissolved in diethyl ether (1 mL), and citric acid monohydrate (38 mg, 0.18 mmol) in THF (0.3 mL) was added. The mixture was stirred at r.t. for 15 min, then the supernatant was decanted and the residue was washed with diethyl ether (3×1 mL), suspended in diethyl ether (2 mL) and concentrated in vacuo (×3), co-evaporated with toluene (1 mL) then diethyl ether (1 mL), to yield a colourless solid (75 mg, 90%). ¹H NMR (400 MHz, ds-DMSO): 7.68 (d, J=8.7 Hz, 1H), 7.41 (d, J=8.7 Hz, 1H), 4.53 (t, J=5.5 Hz, 2H), 3.85 (s, 3H), 3.01 (t, J=5.5 Hz, 2H), 2.66 (d, J=15.3H, 2H), 2.57 (d, J=15.3, 2H), 2.48 (bs, 6H); ¹³C NMR (101 MHz, ds-DMSO): 175.7, 171.3, 163.6, 153.1, 132.7, 129.5, 128.6, 126.4, 126.1, 71.8, 62.3, 55.8, 44.1, 43.5.

### 2-(dimethylammonium)ethyl 3,6-dichloro-2-methoxybenzoate dihydrogenphosphate 1.H₃PO₄

2-(Dimethylamino)ethyl 3,6-dichloro-2-methoxybenzoate (50 mg, 0.17 mmol) was dissolved in diethyl ether (0.5 mL), and orthophosphoric acid (85%, 22 mg, 0.19 mmol) in diethyl ether (0.5 mL) was added. The mixture was stirred at r.t. for 15 min, then the supernatant was decanted and the residue was washed with diethyl ether (3×1 mL), suspended in diethyl ether (1 mL) and concentrated in vacuo (×3), co-evaporated with toluene (3×2 mL), to yield a colourless solid (56 mg, 84%). ¹H NMR (400 MHz, ds-DMSO): 7.67 (d, J=8.7 Hz, 1H), 7.40 (d, J=8.7 Hz, 1H), 4.44 (t, J=5.5 Hz, 2H), 3.84 (s, 3H), 2.74-2.70 (m, 2H), 2.28 (bs, 6H); ¹³C NMR (101 MHz, ds-DMSO): 163.6, 153.1, 132.6, 129.7, 128.6, 126.3, 126.1, 62.8, 62.2, 56.1, 44.4.

Alternative Synthesis of 1

In an alternative synthesis of 1, FIG. 4 provides a scheme for the synthesis of 2-(dimethylamino)ethyl 3,6-dichloro-2-methoxybenzoate 1 from the readily available commercial pesticide dicamba.

To a solution of dicamba (500 mg, 2.26 mmol) and 2-chloro-N,N-dimethylethylamine hydrochloride (218 mg, 1.51 mmol) in 1,4-dioxane (12 ml) was added potassium carbonate (1.25 g, 9.06 mmol). The mixture was stirred at 90° C. for 30 min and then stirred at room temperature for 16 h. The mixture was then transferred to a separating funnel and diluted with water (15 ml) and extracted with CH₂Cl₂ (3×10 ml). The extract was washed with sat. NaHCO₃ (15 ml), brine (15 ml), dried (MgSO₄), filtered and concentrated. The resultant residue was then purified by flash column chromatography (MeOH:CHCl₃ 3:97) to obtain 2-(dimethylamino)ethyl 3,6-dichloro-2-methoxybenzoate 1 as a pale yellow oil (336 mg, 51%).

Herbicidal Activity Assay

A range of compounds of the invention were assessed for herbicidal activity against A. thaliana. A. thaliana Col-0 seeds (˜30) were sown in 63×63×59 mm pots consisting of Irish peat that was pre-wet prior to sowing. Seeds were cold-treated for 3 days in the dark at 4° C. to synchronise germination and then grown in a chamber at 22° C., with 60% relative humidity and in a 16 h light/8 h dark photoperiod. Compounds of the invention were initially dissolved in dimethyl sulfoxide (DMSO) at 20 mg/mL and further diluted in water prior to treatments. The surfactant Brushwet (SST Australia) was added to a final concentration of 0.02%. The carrier DMSO was used as a negative control. Seeds or seedlings were treated with solutions of compound that contained 2% of DMSO, using a pipette. Post-emergence treatments were done at three and six days after germination. Seedlings were grown for 16 days after treatment before photos were taken.

The results of this study are represented in the following table, where compounds of the invention are represented by number, and herbicidal activity is represented using the following: ≤125 μM=+++, ≤500 μM=++, ≤2000 μM=+

| Compound | Activity | Compound | Activity |
|---|---|---|---|
| 1 | ++ | 1. HCl | +++ |
| 2 | +++ | 2. HCl | +++ |
| 3 | ++ | 3. HCl | +++ |
| 4 | +++ | 4. HCl | +++ |
| 5 | ++ | 5. HCl | +++ |
| 6 | + | 6. HCl | + |
| 7 | +++ | 7. HCl | +++ |
| 8 | + | 8. HCl | +++ |
| 9 | + | 9. HCl | + |
| 10 | ++ | 10. HCl | +++ |
| 11 | +++ | 11. HCl | ++ |
| 12 | + | 12. HCl | +++ |
| | | 13. HCl | +++ |
| 14 | +++ | 14. HCl | +++ |
| 15 | ++ | 15. HCl | + |
| 16 | ++ | 16. HCl | +++ |
| 17 | + | 17. HCl | +++ |
| 18 | +++ | 18. HCl | +++ |
| 19 | ++ | 19. HCl | + |
| 20 | ++ | 20. HCl | ++ |
| 21 | ++ | 21. HCl | + |
| 22 | + | 22. HCl | + |
| 23 | +++ | 23. HCl | +++ |
| 24 | ++ | 24. HCl | +++ |
| 25 | + | 25. HCl | + |
| 26 | +++ | 26. HCl | +++ |
| 27 | +++ | 27. HCl | ++ |
| 28 | +++ | 28. HCl | +++ |
| 1. HBr | +++ | | |
| 1. H₂SO₄ | +++ | | |
| 1. TsOH | +++ | | |
| 1. citrate | +++ | | |
| 1. H₃PO₄ | +++ | | |

Volatility

A compound of the invention in the form of 2-(dimethylamino)ethyl 3,6-dichloro-2-methoxybenzoate hydrochloride was compared to the known auxin herbicide dicamba (3,6-dichloro-2-methoxybenzoic acid).

A small-scale drift assay was established. Pots (63×63×59 mm) were filled with Seedling Substrate Plus+ soil (Bord na Móna Horticulture Ltd, Newbridge, Ireland) consisting of Irish peat. Soil was pre-wet before sowing to saturation and a plastic lid from a 50 mL tube placed in the centre of each pot. Seeds of wild type Col-0 Arabidopsis thaliana were sown around each of the lids. Seeds were cold-treated for 3 days in the dark at 4° C. to synchronize germination and then grown in a chamber at 22° C., with 60% relative humidity and in a 16 h light/8 h dark photoperiod. Either dicamba or 2-(dimethylamino)ethyl 3,6-dichloro-2-methoxybenzoate hydrochloride was dissolved in 0.85 mL of water with 0.2% surfactant Brushwet (SST Dandenong, Australia) and added to the lid at one of three concentrations (5, 10 or 15 mM). After growth for 2 weeks plants were imaged. (In the case of FIG. 27, images were recorded at 17 days).

As can be seen in FIG. 5, burn symptoms are apparent in dicamba (top row). By contrast no drift burn at all was visible for 2-(dimethylamino)ethyl 3,6-dichloro-2-methoxybenzoate hydrochloride (bottom row).

A second experiment was performed using the same methodology, but at higher concentrations (15 mM and 30 mM). As can be seen in FIG. 6, again, the compound of the invention (right column) did not show any drift burn, where dicamba (left column) did.

Activity Vs a Panel of Plants

The inventors have determined that the illustrative compound of Formula (A) has activity similar against a range of plants to the known auxin herbicide dicamba on an equimolar basis.

Pots (63×63×59 mm) were filled with Seedling Substrate Plus+ soil (Bord na Móna Horticulture Ltd, Newbridge, Ireland) consisting of Irish peat. Soil was pre-wet to saturation before sowing seeds. Seeds of the following species were sown.
Arabidopsis thaliana
Cynodon dactylon (couch grass)
Eragrostis tef (tef)
Lolium rigidum (herbicide resistant ryegrass)
Ratibida columnifera (prairie coneflower)
Raphanus raphanistrum (wild radish)
Solanum lycopersicum (tomato)
Triticum aestivum (wheat)

The number of seeds depended on germination rate and plant size. Seeds were sown in pots (63×63×59 mm) of Seedling Substrate Plus+ soil (Bord na Móna Horticulture Ltd, Newbridge, Ireland) consisting of Irish peat. Soil was pre-wet before sowing to saturation and watered throughout the experiment to maintain adequate moisture. No fertilizer was added. Seeds were cold-treated 7 days in the dark at 4° C. to synchronize germination and grown in a chamber at 22° C., with 60% relative humidity and a 16 h light/8 h dark photoperiod.

Dicamba and 2-(dimethylamino)ethyl 3,6-dichloro-2-methoxybenzoate hydrochloride were initially dissolved in DMSO at 20 mg/mL and further diluted in water prior to treatments to a final concentration of 2% DMSO. The surfactant Brushwet (SST Dandenong, Australia) was added to a final concentration of 200 μL/L (0.02%). The negative control contained DMSO and Brushwet at the concentrations described.

Each compound was tested on seedlings by pipetting 600 μL of 0, 31.25, 62.5, 125, 250, 500, 1000, 2000 or 4000 μmol/L solutions directly onto the plants; these quantities and concentrations are equivalent to ˜10, 20, 40, 78, 156, 312, 625 and 1250 g/ha a.i. (grams of active ingredient per hectare) for dicamba and ˜15, 29, 58, 116, 233, 465, 930 and 1860 g/ha a.i for 2-(dimethylamino)ethyl 3,6-dichloro-2-methoxybenzoate hydrochloride.

The soil density in each pot was ˜0.29 g/cm³, based on dry soil, and the total volume of herbicide solution per gram of soil was 24 μL/g.

The day the seedlings were treated was considered as day 0. Post-emergence treatments were carried out twice post germination, at days 0 and 3, and seedlings were grown for 10-12 days before photographs were taken.

Results for each species are presented below.

Arabidopsis thaliana

Once the Arabidopsis thaliana seeds had germinated, this was Day 0 and an image taken (Treatment—Day 0) to show plants were all at the same stage. Plants were treated either with dicamba or 2-(dimethylamino)ethyl 3,6-dichloro-2-methoxybenzoate hydrochloride at concentrations ranging from 31.25 μM to 4000 μM as marked and imaged 12 days later (Treatment—Day 12). Some plants were not treated with either dicamba or 2-(dimethylamino)ethyl 3,6-dichloro-2-methoxybenzoate hydrochloride (No treatment). These were negative controls (−ve controls) and imaged at the same point before and after treatment. FIG. 7 shows that, on an equimolar basis, dicamba and 2-(dimethylamino)ethyl 3,6-dichloro-2-methoxybenzoate hydrochloride have comparable activity against Arabidopsis thaliana.

Cynodon dactylon (couch grass)

Once the Cynodon dactylon seeds had germinated, this was Day 0 and an image taken (Treatment—Day 0) to show plants were all at the same stage. Plants were treated either with dicamba or 2-(dimethylamino)ethyl 3,6-dichloro-2-methoxybenzoate hydrochloride at concentrations ranging from 31.25 μM to 4000 μM as marked and imaged 10 days later (Treatment—Day 10). Some plants were not treated with either dicamba or 2-(dimethylamino)ethyl 3,6-dichloro-2-methoxybenzoate hydrochloride (No treatment). These were negative controls (−ve controls) and imaged at the same point before and after treatment. This FIGS. 8 and 9 show that, on an equimolar basis, dicamba and 2-(dimethylamino)ethyl 3,6-dichloro-2-methoxybenzoate hydrochloride have comparable activity against Cynodon dactylon.

Eragrostis tef (tef)

Once the Eragrostis tef seeds had germinated, this was Day 0 and an image taken (Treatment—Day 0) to show plants were all at the same stage. Plants were treated either with dicamba or 2-(dimethylamino)ethyl 3,6-dichloro-2-methoxybenzoate hydrochloride at concentrations ranging from 31.25 μM to 4000 μM as marked and imaged 12 days later (Treatment—Day 12). Some plants were not treated with either dicamba or 2-(dimethylamino)ethyl 3,6-dichloro-2-methoxybenzoate hydrochloride (No treatment). These were negative controls (−ve controls) and imaged at the same point before and after treatment. FIG. 10 shows that, on an equimolar basis, dicamba and 2-(dimethylamino)ethyl 3,6-dichloro-2-methoxybenzoate hydrochloride have comparable activity against Eragrostis tef.

Lolium rigidum (herbicide resistant ryegrass)

Once the Lolium rigidum seeds had germinated, this was Day 0 and an image taken (Treatment—Day 0) to show plants were all at the same stage. Plants were treated either with dicamba or 2-(dimethylamino)ethyl 3,6-dichloro-2-methoxybenzoate hydrochloride at concentrations ranging from 31.25 μM to 4000 μM as marked and imaged 12 days later (Treatment—Day 12). Some plants were not treated with either dicamba or 2-(dimethylamino)ethyl 3,6-dichloro-2-methoxybenzoate hydrochloride (No treatment). These were negative controls (−ve controls) and imaged at the same point before and after treatment. FIG. 11 shows that, on an equimolar basis, dicamba and 2-(dimethylamino)ethyl 3,6-dichloro-2-methoxybenzoate hydrochloride have comparable activity against Lolium rigidum.

Ratibida columnifera (prairie coneflower, aka Mexican hat)

Once the Ratibida columnifera seeds had germinated, this was Day 0 and an image taken (Treatment—Day 0) to show plants were all at the same stage. Plants were treated either with dicamba or 2-(dimethylamino)ethyl 3,6-dichloro-2-methoxybenzoate hydrochloride Compound at concentrations ranging from 31.25 μM to 4000 μM as marked and imaged 12 days later (Treatment—Day 12). Some plants were not treated with either dicamba or 2-(dimethylamino)ethyl 3,6-dichloro-2-methoxybenzoate hydrochloride (No treatment). These were negative controls (−ve controls) and imaged at the same point before and after treatment. FIG. 12 shows that, on an equimolar basis, dicamba and 2-(dimethylamino)ethyl 3,6-dichloro-2-methoxybenzoate hydrochloride have comparable activity against Ratibida columnifera.

Raphanus raphanistrum (wild radish)

Once the Raphanus raphanistrum seeds had germinated, this was Day 0 and an image taken (Treatment—Day 0) to show plants were all at the same stage. Plants were treated either with dicamba or 2-(dimethylamino)ethyl 3,6-dichloro-2-methoxybenzoate hydrochloride at concentrations ranging from 31.25 μM to 4000 μM as marked and imaged 10 days later (Treatment—Day 10). Some plants were not treated with either dicamba or 2-(dimethylamino)ethyl 3,6-dichloro-2-methoxybenzoate hydrochloride (No treatment). These were negative controls (−ve controls) and imaged at the same point before and after treatment. FIGS. 13 and 14 show that, on an equimolar basis, dicamba and 2-(dimethylamino)ethyl 3,6-dichloro-2-methoxybenzoate hydrochloride Compound have comparable activity against Raphanus raphanistrum.

Solanum lycopersicum (tomato)

Once the Solanum lycopersicum seeds had germinated, this was Day 0 and an image taken (Treatment—Day 0) to show plants were all at the same stage. Plants were treated either with dicamba or 2-(dimethylamino)ethyl 3,6-dichloro-2-methoxybenzoate hydrochloride at concentrations ranging from 31.25 μM to 4000 μM as marked and imaged 10 days later (Treatment—Day 10). Some plants were not treated with either dicamba or 2-(dimethylamino)ethyl 3,6-dichloro-2-methoxybenzoate hydrochloride (No treatment). These were negative controls (−ve controls) and imaged at the same point before and after treatment. FIGS. 15 and 16 show that, on an equimolar basis, dicamba and 2-(dimethylamino)ethyl 3,6-dichloro-2-methoxybenzoate hydrochloride have comparable activity against Solanum lycopersicum.

Triticum aestivum (wheat)

Once the Triticum aestivum seeds had germinated, this was Day 0 and an image taken (Treatment—Day 0) to show plants were all at the same stage. Plants were treated either with dicamba or 2-(dimethylamino)ethyl 3,6-dichloro-2-methoxybenzoate hydrochloride at concentrations ranging from 31.25 μM to 4000 μM as marked and imaged 12 days later (Treatment—Day 12). Some plants were not treated with either dicamba or 2-(dimethylamino)ethyl 3,6-dichloro-2-methoxybenzoate hydrochloride (No treatment). These were negative controls (−ve controls) and imaged at the same point before and after treatment. FIG. 17 shows that, on an equimolar basis, dicamba and 2-(dimethylamino)ethyl 3,6-dichloro-2-methoxybenzoate hydrochloride have comparable activity against Triticum aestivum.

### Synthesis of 2-(dimethylamino)ethyl 4-(2,4-dichlorophenoxy)butanoate and 2-(dimethylamino)ethyl 4-(2,4-dichlorophenoxy)butanoate hydrochloride

This synthesis is represented in FIG. 18.

### 4-(2,4-dichlorophenoxy)butanoyl chloride

To a mixture of 4-(2,4-dichlorophenoxy)butanoic acid (2,4-DB) (250 mg, 1.0 mmol) in benzene (4 ml) was added DMF (1 drop) and thionyl chloride (0.09 ml, 1.2 mmol) at room temperature and then was stirred at reflux for 4 h. The solution was then concentrated and the resultant residue co-evaporated with toluene (4×10 ml) to give 4-(2,4-dichlorophenoxy)butanoyl chloride (265 mg).

### 2-(dimethylamino)ethyl 4-(2,4-dichlorophenoxy)butanoate hydrochloride

To a solution of 2-dimethylaminoethanol (0.1 ml, 1.0 mmol) in CHCl₃ (2 ml) was added 4-(2,4-dichlorophenoxy)butanoyl chloride (265 mg, 1.0 mmol) in CHCl₃ (2 ml) at 0° C. and the resultant solution stirred at room temperature for 3 h. After this time Et₂O (10 ml) was added and a solid precipitated. Removal of the solvent and trituration of the solid with Et₂O (3×10 ml) gave 2-(dimethylamino)ethyl 4-(2,4-dichlorophenoxy)butanoate hydrochloride as a colourless solid (298 mg, 83% over two steps). ¹H NMR (500 MHz, d₆-DMSO): δ 10.71 (br s, 1H), 7.56 (d, J=2.6 Hz, 1H), 7.36 (dd, J=2.6, 8.8 Hz, 1H), 7.17 (d, J=8.8 Hz, 1H), 4.39-4.36 (m, 2H), 4.10 (app t, J=6.3 Hz, 2H), 3.37-3.33 (m, 2H), 2.76 (s, 6H), 2.57 (app t, J=7.3 Hz, 2H), 2.02 (app quint, J=7.2 Hz, 2H); ¹³C NMR (125.8 MHz, d₆-DMSO): δ 172.1, 152.9, 129.3, 128.2, 124.5, 122.4, 115.1, 68.0, 58.4, 54.8, 42.4, 30.0, 23.8.

### 2-(dimethylamino)ethyl 4-(2,4-dichlorophenoxy)butanoate

To a solution of 2-(dimethylamino)ethyl 4-(2,4-dichlorophenoxy)butanoate hydrochloride (130 mg, 0.36 mmol) in CHCl₃ (2 ml) was added Et₃N (0.053 ml, 0.38 mmol) and the resultant solution stirred at room temperature for 10 min. The mixture was then concentrated and then taken up in hexane (10 ml) and the resultant solid filtered. The hexane solution was then washed with water (3×5 ml), dried (MgSO₄), filtered and concentrated to give 2-(dimethylamino)ethyl 4-(2,4-dichlorophenoxy)butanoate as a colourless oil (100 mg, 86%). ¹H NMR (500 MHz, d₆-DMSO): δ 7.55 (d, J=2.6 Hz, 1H), 7.36 (dd, J=2.6, 8.8 Hz, 1H), 7.16 (d, J=8.8 Hz, 1H), 4.11-4.07 (m, 4H), 2.48 (app t, J=7.3 Hz, 2H), 2.44 (app t, J=5.8 Hz, 2H), 2.13 (s, 6H), 1.98 (app quint, J=6.8 Hz, 2H); ¹³C NMR (125.8 MHz, d₆-DMSO): δ 172.4, 152.9, 129.3, 128.1, 124.4, 122.4, 115.1, 67.9, 61.7, 57.2, 45.3, 30.0, 24.0.

### Activity of 2-(dimethylamino)ethyl 4-(2,4-dichlorophenoxy)butanoate

Once the Arabidopsis thaliana seeds had germinated, this was Day 0 and an image taken (Treatment—Day 0) to show plants were all at the same stage. Plants were treated either with 2,4-DB or 2-(dimethylamino)ethyl 4-(2,4-dichlorophenoxy)butanoate at concentrations ranging from 31.25 μM to 2000 μM as marked and imaged 12 days later (Treatment—Day 12). Some plants were not treated with either 2,4-DB or 2-(2-(dimethylamino)ethyl 4-(2,4-dichlorophenoxy)butanoate. These were negative controls (−ve controls) and imaged at the same point before and after treatment. FIG. 19 shows that, on an equimolar basis, 2,4-DB and 2-(dimethylamino)ethyl 4-(2,4-dichlorophenoxy)butanoate have comparable activity against Arabidopsis thaliana.

### Activity of and 2-(dimethylamino)ethyl 4-(2,4-dichlorophenoxy)butanoate hydrochloride

Once the Arabidopsis thaliana seeds had germinated, this was Day 0 and an image taken (Treatment—Day 0) to show plants were all at the same stage. Plants were treated either with 2,4-DB or 2-(dimethylamino)ethyl 4-(2,4-dichlorophenoxy)butanoate hydrochloride at concentrations ranging from 31.25 μM to 2000 μM as marked and imaged 12 days later (Treatment—Day 12). Some plants were not treated with either 2,4-DB or 2-(2-(dimethylamino)ethyl 4-(2,4-dichlorophenoxy)butanoate hydrochloride. These were negative controls (−ve controls) and imaged at the same point before and after treatment. FIG. 20 shows that, on an equimolar basis, 2,4-DB and 2-(dimethylamino)ethyl 4-(2,4-dichlorophenoxy)butanoate hydrochloride have comparable activity against Arabidopsis thaliana.

### Synthesis of 3,6-dichloropyridine-2-carboxylic acid 2-(dimethylamino)ethyl ester and 3,6-dichloropyridine-2-carboxylic acid 2-(dimethylamino)ethyl ester hydrochloride

This synthesis is represented in FIG. 21.

### 3,6-dichloropyridine-2-carboxylic acid 2-(dimethylamino)ethyl ester

Clopyralid (200 mg, 1.04 mmol) was suspended in CH₂Cl₂ (4 mL), and DMF (1 drop) was added, followed by oxalyl chloride (0.13 mL, 1.5 mmol). The mixture was stirred at r.t. for 2 hrs, then concentrated in vacuo, and co-evaporated with toluene (2×10 mL). The residue was taken up in CH₂Cl₂ (3 mL) and 2-dimethylaminoethanol (0.21 mL, 2.1 mmol) was added. The mixture was stirred at r.t. for 18 hr, then quenched with saturated NaHCO₃ solution (2 mL). The aqueous phase was separated and extracted with CH₂Cl₂ (3×2 mL). The combined organic phases were washed with water (2×10 mL), dried over MgSO₄, filtered and concentrated, then purified by silica gel chromatography (0-10% MeOH/CH₂Cl₂) to yield a colourless oil which solidified on standing (218 mg, 80%). ¹H NMR (400 MHz, CDCl₃): 7.73 (d, J=8.5 Hz, 1H), 7.38 (d, J=8.5 Hz, 1H), 4.49 (t, J=5.9 Hz, 2H), 2.73 (t, J=5.9 Hz, 2H), 2.32 (s, 6H); ¹³C NMR (101 MHz, CDCl₃): 163.4, 149.1, 147.8, 141.1, 129.7, 127.4, 64.1, 57.5, 45.8.

### 3,6-dichloropyridine-2-carboxylic acid 2-(dimethylamino)ethyl ester hydrochloride

3,6-Dichloropyridine-2-carboxylic acid 2-(dimethylamino)ethyl ester (95 mg, 0.36 mmol) was dissolved in diethyl ether (2 mL) and 2M HCl in diethyl ether (0.2 mL, 0.4 mmol) was added. The mixture was concentrated in vacuo and co-evaporated in toluene to give a white powder (106 mg, 98%). ¹H NMR (400 MHz, ds-DMSO): 10.64 (bs, 1H), 8.23 (d, J=8.6 Hz, 1H), 7.83 (d, J=8.6 Hz, 1H), 4.74-4.71 (m, 2H), 3.54-3.51 (m, 2H), 2.83 (s, 6H); ¹³C NMR (101 MHz, ds-DMSO): 162.0, 148.0, 145.8, 142.7, 129.5, 128.7, 60.6, 54.5, 42.7.

### Synthesis of 3,7-dichloroquinoline-8-carboxylic acid 2-(dimethylamino)ethyl ester and 3,7-dichloroquinoline-8-carboxylic acid 2-(dimethylamino)ethyl ester hydrochloride

This synthesis is represented in FIG. 22.

### 3,7-dichloroquinoline-8-carboxylic acid 2-(dimethylamino)ethyl ester

Quinclorac (252 mg, 1.04 mmol) was suspended in CH₂Cl₂ (4 mL), and DMF (1 drop) was added, followed by oxalyl chloride (0.13 mL, 1.5 mmol). The mixture was stirred at r.t. for 1.5 hrs, then concentrated in vacuo, and co-evaporated with toluene (2×10 mL). The residue was taken up in CH₂Cl₂ (3 mL) and 2-dimethylaminoethanol (0.21 mL, 2.1 mmol) was added. The mixture was stirred at r.t. for 18 hr, then quenched with saturated NaHCO₃ solution (3 mL). The aqueous phase was separated and extracted with CH₂Cl₂ (3×3 mL). The combined organic phases were washed with water (10 mL), dried over MgSO₄, filtered and concentrated, then purified by silica gel chromatography (0-10% MeOH/CH₂Cl₂) to yield a yellow solid (128 mg, 39%). ¹H NMR (400 MHz, CDCl₃): 8.83 (d, J=2.4 Hz, 1H), 8.13 (d, J=2.4 Hz, 1H), 7.74 (d, J=8.8 Hz, 1H), 7.56 (d, J=8.8 Hz, 1H), 4.65 (t, J=5.8 Hz, 2H), 2.81 (t, J=5.8 Hz, 2H), 2.36 (s, 6H); ¹³C NMR (101 MHz, CDCl₃): 166.1, 151.2, 143.9, 133.9, 133.1, 132.1, 129.6, 129.0, 129.0, 126.8, 63.8, 57.6, 45.7.

### 3,7-dichloroquinoline-8-carboxylic acid 2-(dimethylamino)ethyl ester hydrochloride

3,7-Dichloroquinoline-8-carboxylic acid 2-(dimethylamino)ethyl ester (95 mg, 0.36 mmol) was dissolved in 1:1 CH₂Cl₂/diethyl ether (3 mL) and 2M HCl in diethyl ether (0.14 mL, 0.28 mmol) was added. The resulting precipitate was filtered, washed with diethyl ether (3×1 mL), and air dried to yield a light yellow solid (72 mg, 84%). ¹H NMR (400 MHz, ds-DMSO): 10.28 (bs, 1H), 9.00 (d, J=2.5 Hz, 1H), 8.77 (d, J=2.5 Hz, 1H), 8.18 (d, J=8.9 Hz, 1H), 7.87 (d, J=8.9 Hz, 1H), 4.81-8.78 (m, 2H), 3.55-3.52 (m, 2H), 2.82 (s, 6H); ¹³C NMR (101 MHz, d₆-DMSO): 164.8, 151.2, 142.8, 134.9, 131.3, 130.9, 130.7, 128.9, 128.7, 126.7, 60.5, 54.4, 42.6.

### Synthesis of N-(2,6-dimethylphenyl)-N-(methoxyacetyl)-DL-alanine 2-(dimethylamino)ethyl ester and N-(2,6-dimethylphenyl)-N-(methoxyacetyl)-DL-alanine 2-(dimethylamino)ethyl ester hydrochloride

This synthesis is represented in FIG. 23.

### N-(2,6-dimethylphenyl)-N-(methoxyacetyl)-DL-alanine 2-(dimethylamino)ethyl ester

Metalaxyl (250 mg, 0.895 mmol), LiOH (90 mg, 3.6 mmol), methanol (2 mL) and water (2 mL) were stirred at r.t. for 24 h, then acidified with 1M HCl (4 mL), extracted with CH₂Cl₂ (4×5 mL), dried over MgSO₄, filtered and concentrated. To the residue was added 2-chloro-N,N-dimethylethylamine hydrochloride (129 mg, 0.896 mmol), potassium carbonate (725 mg, 5.25 mmol) and dioxane (10 mL) and the mixture was heated at 90° C. for 3 h, then concentrated in vacuo. The residue was partitioned between water (5 mL) and CH₂Cl₂ (5 mL), and the aqueous phase was extracted with CH₂Cl₂ (2×5 mL). The combined organic phases were dried over MgSO₄, filtered and concentrated, then purified by silica gel chromatography (0-20% MeOH/CH₂Cl₂) to yield a colourless oil (123 mg, 42%). ¹H NMR (400 MHz, CDCl₃): 7.23-7.19 (m, 1H), 7.16-7.14 (m, 1H), 7.11-7.09 (m, 1H), 4.53 (q, J=7.4 Hz, 1H), 4.38-4.33 (m, 1H), 4.29-4.23 (m, 1H), 3.59 (d, J=15.4 Hz, 1H), 3.48 (d, J=15.4 Hz, 1H), 3.32 (s, 3H), 2.72-2.60 (m, 2H), 2.47 (s, 3H), 2.31 (s, 6H), 2.14 (s, 3H), 0.99 (d, J=7.4 Hz, 3H); ¹³C NMR (101 MHz, CDCl₃): 172.5, 170.1, 138.9, 137.6, 135.5, 129.5, 129.2, 128.8, 70.7, 62.9, 59.4, 57.7, 55.4, 45.8, 18.7, 18.3, 14.8.

### N-(2,6-dimethylphenyl)-N-(methoxyacetyl)-DL-alanine 2-(dimethylamino)ethyl ester hydrochloride

To a solution of N-(2,6-dimethylphenyl)-I-(methoxyacetyl)-DL-alanine 2-(dimethylamino)ethyl ester (83 mg, 0.25 mmol) in diethyl ether (2 mL) was added 2M HCl in diethyl ether (0.16 mL, 0.32 mmol) to produce a white precipitate. The supernatant was decanted and the residue was washed with diethyl ether (1 mL), then dried under reduced pressure to yield a white solid (90 mg, 98%). ¹H NMR (400 MHz, ds-DMSO): 10.84 (bs, 1H), 7.28-7.19 (m, 3H), 4.53-4.41 (m, 3H), 3.52 (d, J=15.5 Hz, 1H), 3.45-3.40 (m, 3H), 3.17 (s, 3H), 2.82 (s, 6H), 2.38 (s, 3H), 2.13 (s, 3H), 0.94 (d, J=7.3 Hz, 3H); ¹³C NMR (101 MHz, ds-DMSO): 171.4, 169.3, 137.9, 137.4, 135.1, 129.1, 129.0, 128.8, 69.7, 59.2, 58.4, 54.8, 54.6, 42.5, 18.0, 17.8, 14.2.

### Synthesis of 2-(Dimethylamino)ethyl 2-[4,5-dihydro-4-methyl-4-(1-methylethyl)-5-oxo-1H-imidazol-2-yl]-3-pyridinecarboxylate hydrochloride

This synthesis is represented in FIG. 24.

### 2-(Dimethylamino)ethyl 2-[4,5-dihydro-4-methyl-4-(1-methylethyl)-5-oxo-1H-imidazol-2-yl]-3-pyridinecarboxylate hydrochloride

2-Isopropyl-2-methyl-5H-imidazo-[1′,2′:1,2]pyrrolo[3,4-b]pyridine-3,5(2H)-dione (220 mg, 0.904 mmol) (synthesis described in U.S. Pat. No. 4,798,619), 2-dimethylaminoethanol (0.18 mL, 1.8 mmol) and toluene (1.5 mL) were heated at 50° C. for 1 hr, then at 90° C. for 1.5 hrs. The solution was concentrated, purified by silica gel chromatography (0-10% MeOH/CH₂Cl₂) to yield a white solid, which was dissolved in CH₂Cl₂ (2 mL) and 2M HCl in diethyl ether (0.28 mL, 0.56 mmol) was added. The mixture was concentrated, suspended in CH₂Cl₂ (2 mL) and precipitated with diethyl ether (2 mL). The supernatant was decanted, and the residue was washed with diethyl ether (2×1 mL) and dried to yield a hygroscopic white solid (142 mg, 43%). ¹H NMR (600 MHz, ds-DMSO): 10.54 (bs, 1H), 8.86 (dd, J=4.8, 1.1 Hz, 1H), 8.33 (dd, J=7.8, 1.1 Hz, 1H), 7.76 (dd, J=7.8, 4.8 Hz, 1H), 4.64-4.60 (m, 1H), 4.55-4.52 (m, 1H), 3.51-3.49 (m, 2H), 2.81 (s, 6H), 1.92 (sept, J=6.7 Hz, 1H), 1.25 (s, 3H), 0.96 (d, J=6.7 Hz, 3H), 0.78 (d, J=6.7 Hz, 3H); ¹³C NMR (151 MHz, ds-DMSO): 187.1, 165.9, 159.5, 151.0, 144.7, 137.1, 128.5, 125.9, 73.9, 60.0, 54.6, 42.5, 34.2, 20.6, 16.8, 16.7.

### Synthesis of 2-(Dimethylamino)ethyl 2-[1-[2-[[(3-fluorophenyl)amino]carbonyl]hydrazinylidene]ethyl]-3-pyridinecarboxylate hydrochloride

This synthesis is represented in FIG. 25.

### 2-(Dimethylamino)ethyl 2-[1-[2-[[(3-fluorophenyl)amino]carbonyl]hydrazinylidene]ethyl]-3-pyridinecarboxylate hydrochloride

Diflufenzopyr (100 mg, 0.299 mmol), 2-chloro-N,N-dimethylethylamine hydrochloride (58 mg, 0.40 mmol), potassium carbonate (253 mg, 1.83 mmol) and dioxane (5 mL) were heated at 90° C. for 1.5 hr, then filtered and concentrated. The residue was purified by silica gel chromatography (0-10% MeOH/CH₂Cl₂) to yield a white solid, which was dissolved in CH₂Cl₂ (1 mL) and 2M HCl in diethyl ether (60 μL, 0.12 mmol) was added to produce a white precipitate, which was filtered, washed with diethyl ether (2×1 mL), then dried under reduced pressure to yield a white solid (28 mg, 23%). ¹H NMR (400 MHz, ds-DMSO): 10.56 (s, 1H), 10.21 (bs, 1H), 9.42 (bs, 1H), 8.75-8.74 (m, 1H), 8.10-8.08 (m, 1H), 7.57-7.54 (m, 1H), 7.31-7.29 (m, 2H), 6.87-6.83 (m, 1H), 4.52-4.49 (m, 2H), 3.41-3.38 (m, 2H), 2.73 (s, 6H), 2.38 (s, 3H); ¹³C NMR (151 MHz, ds-DMSO): 168.5, 162.7 (dd, J=16, 243 Hz), 153.1, 152.4, 150.2, 146.7, 141.8 (dd, J=14, 14 Hz), 136.5, 127.1, 123.2, 101.3 (d, J=29 Hz), 97.5 (dd, J=26, 26 Hz, 1H), 59.9, 54.7, 42.5, 13.7.

Herbicidal Activity Assay

A. thaliana Col-0 seeds (˜30) were sown in 63×63×59 mm pots consisting of Irish peat that was pre-wet prior to sowing. Seeds were cold-treated for 3 days in the dark at 4° C. to synchronise germination and then grown in a chamber at 22° C., with 60% relative humidity and in a 16 h light/8 h dark photoperiod. Compounds were initially dissolved in dimethyl sulfoxide (DMSO) at 20 mg/mL and further diluted in water prior to treatments. The surfactant Brushwet (SST Australia) was added to a final concentration of 0.02%. The carrier DMSO was used as a negative control. Seeds or seedlings were treated with solutions of compound that contained 2% of DMSO, using a pipette. Post-emergence treatments were done at three and six days after germination. Seedlings were grown for 16 days after treatment before photos were taken.

| Compound | Active at |
|---|---|
| 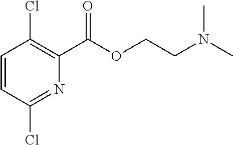 [SMILES: CN(C)CCOC(=O)c1nc(Cl)ccc1Cl] | 125 μM |
| 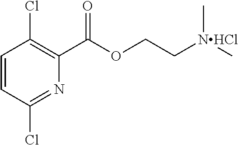 | 125 μM |
| 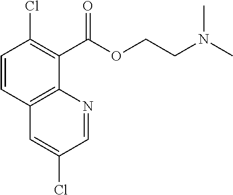 [SMILES: CN(C)CCOC(=O)c1c(Cl)ccc2cc(Cl)cnc12] | 2000 μM |
| 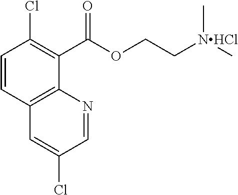 | 2000 μM |
| 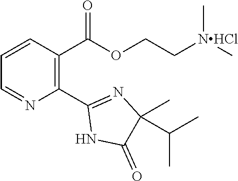 | 500 μM |
| 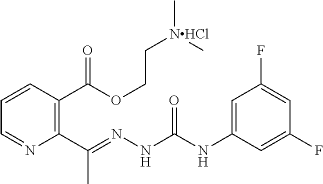 | 125 μM |

Creating a Dicamba Tolerant A. thaliana Line

To recreate dicamba tolerance by dicamba monooxygenase in A. thaliana similarly to that made by Behrens et al. (2007, Science 316: 1185-1188) a synthetic gene encoding the oxygenaseDIC from Stenotrophomonas maltophilia strain DI-6 (GenBank accession AY786443) was made to include a 58-residue chloroplast targeting leader used by Anderson and Smith (1986, Biochem. J. 240: 709-715) and incorporated a Trp to Cys mutation said to provide useful changes in pH and temperature optima (See, for example, W112C in International Patent Application WO2007146706A2). A BamHI site immediately preceding the sequence encoding the start Met and a SacI site immediately after the stop codon were used to subclone this synthetic DMOc gene into pMDC43 (Curtis and Grossniklaus 2003 Plant Physiol. 133: 462-469), from which the mGFP6 coding sequence and Gateway recombination cassette were removed by the same restriction enzymes, putting DMOc under control of a double CaMV35S promoter and providing it the nopaline synthase terminator. The plasmid pMDC43-DMOc was transferred from E. coli containing the plasmid to Agrobacterium tumefaciens (strain LBA4404) using a helper strain (E. coli H3101 pRK2013). Wild type Col-0 Arabidopsis thaliana was transformed by in planta transformation. The first transgenic generation (T₁) was selected on MS-agar plates (1% agar, 1×MS salts, pH 5.7, 1% glucose) containing 20 μg/mL hygromycin. Dicamba resistance of their progeny (T₂) was used to identify lines segregating for resistance as a single T-DNA locus. Homozygous T₃ seeds were identified by hygromycin resistance on plates and then confirmed by dicamba resistance on soil.

Demonstrating 1·HCl is Compatible with the DMOc Trait

To test the conferral of dicamba tolerance to 1·HCl by the DMOc transgene approximately 40 seeds of wild type Col-0 A. thaliana or T3 homozygous seeds for a single-locus DMOc line were sown on a series of 6 soil pots. After 4 days stratification, pots were moved to 23° C. and 16 hour days. Seedlings were not sprayed (0 μM), sprayed with dicamba (at 7, 15, 31, 62, or 125 μM) or 1·HCl (at 31, 62, 125, 250, or 500 μM) and 0.02% Brushwet at 4 and 7 days after being moved to grow in the light. The volume sprayed per pot was 750 μL. Plants were imaged at Day 11 of growth and the results are shown in FIG. 26.

Drift of Other Compounds of Formula (B)

FIG. 27 compares the drift behaviour of certain compounds of Formula (B) (being the hydrochloride salts of compounds 3, 5, 12, 18, 27) to dicamba, using the materials and methods described in the context of FIGS. 5 and 6. The 1·HCl compound is included as a control as it does not drift, as demonstrated above. As is evident from the leaf curling at 2.5 mM dicamba drifted ‘south-east’ (arrows).

1. A compound of Formula (A); 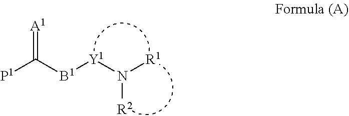
wherein;
dotted lines indicate that the indicated atoms may form part of a cyclic structure;
P¹ is selected from the group consisting of; 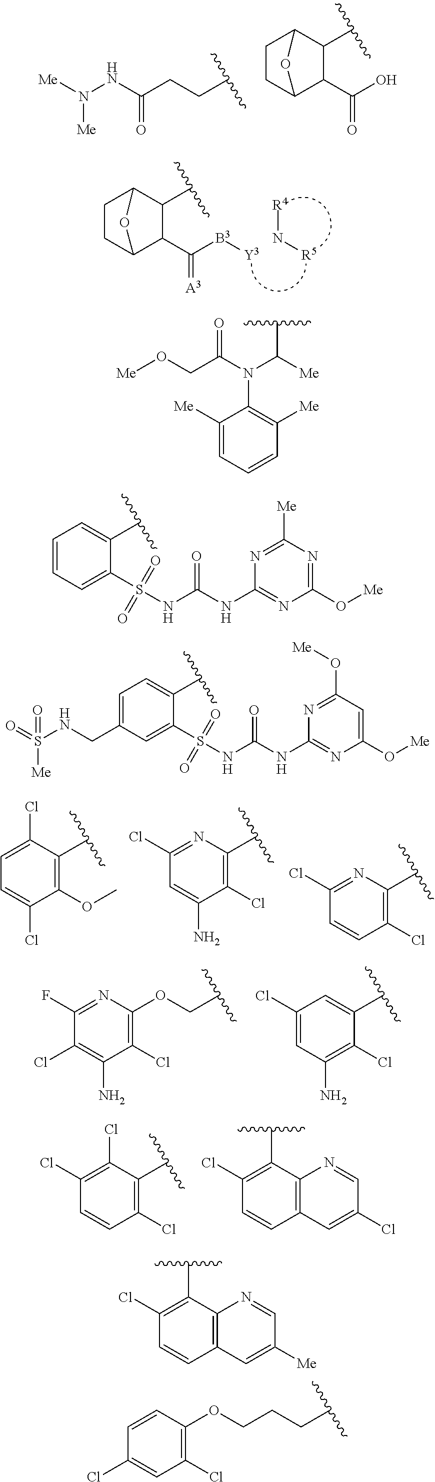 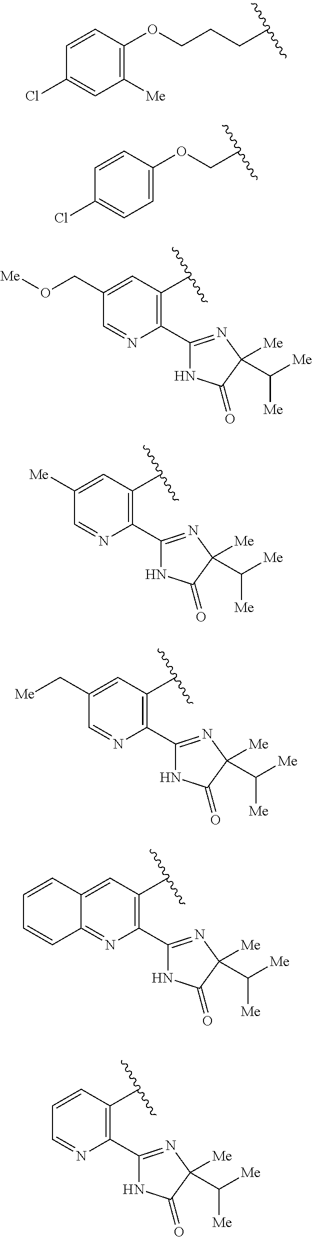 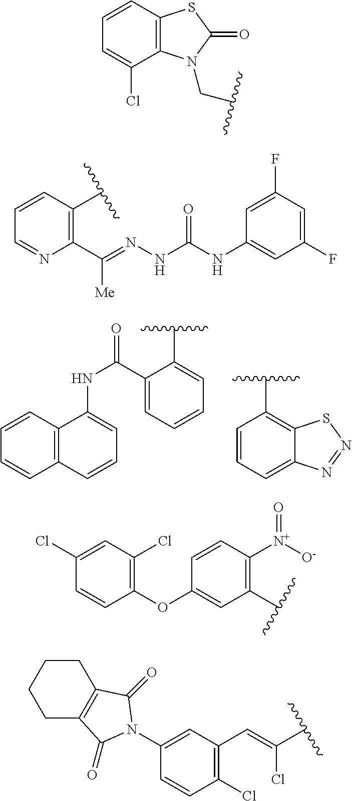
A¹ is O or S;
B¹ is O, S or N;
Y¹ is C₂-C₉ alkyl, straight, branched or cyclic, unsubstituted or substituted by halogen, alkoxy, cyano, hydroxyl, alkylamine or aryl, may be interrupted by O, S or >NR³;
R¹ is H; aryl; C₁-C₆ alkyl, straight, branched or cyclic, unsubstituted or substituted by halogen, alkoxy, cyano, hydroxyl, alkylamine or aryl, and may be interrupted by one or more of O, S or >NR₃, provided that any multiple interruptions are not consecutive;
R³ is C₁-C₆ alkyl;
R² is H; aryl; C₁-C₆ alkyl, straight, branched or cyclic, unsubstituted or substituted by halogen, alkoxy, cyano, hydroxyl, alkylamine or aryl, and may be interrupted by one or more of O, S or >NR³ provided that any multiple interruptions are not consecutive;
or R² is 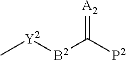
and wherein;
P² is, independently of P¹ selected from the group consisting of; 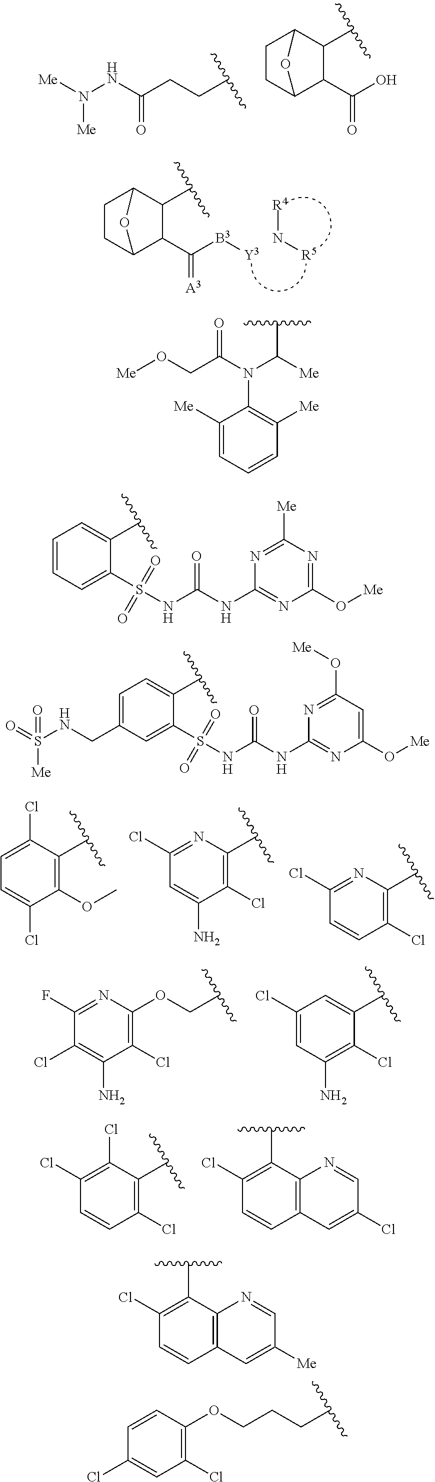 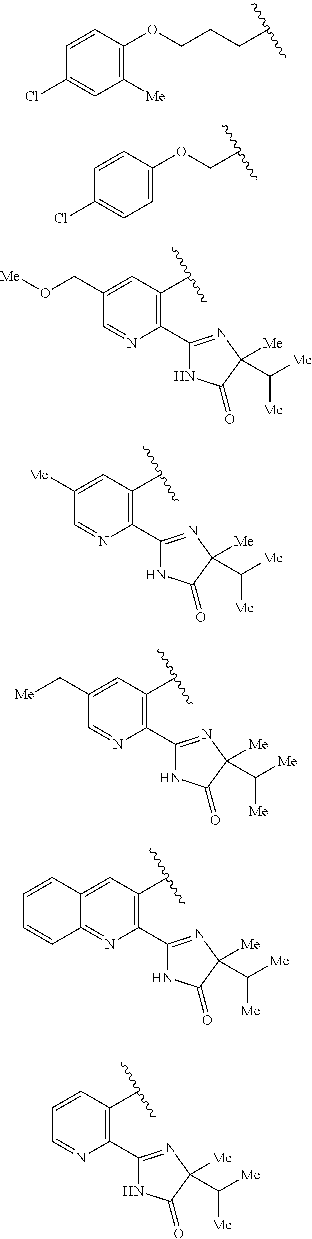 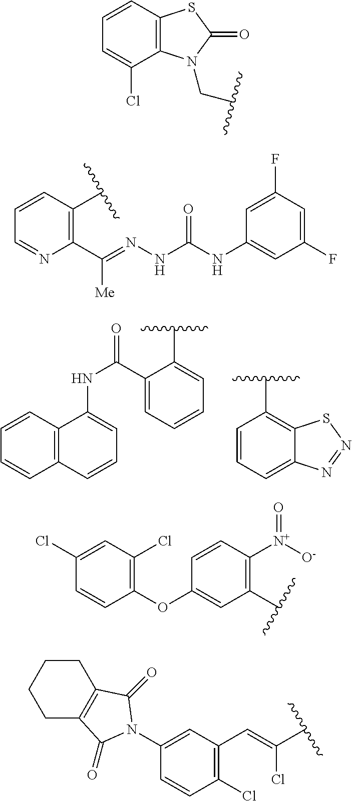
A² is O or S;
B² is O, S or N;
Y² is C₂-C₉ alkyl, straight, branched or cyclic, unsubstituted or substituted by halogen, alkoxy, cyano, hydroxyl, alkylamine or aryl, may be interrupted by one or more of O, S or >NR₃ provided that any multiple interruptions are not consecutive;
and wherein;
A³ is O or S;
B³ is O, S or N;
Y³ is C₂-C₉ alkyl, straight, branched or cyclic, unsubstituted or substituted by halogen, alkoxy, cyano, hydroxyl, alkylamine or aryl, may be interrupted by O, S or >NR⁶;
R⁴ is H; aryl; C₁-C₆ alkyl, straight, branched or cyclic, unsubstituted or substituted by halogen, alkoxy, cyano, hydroxyl, alkylamine or aryl, and may be interrupted by one or more of O, S or >NR₃, provided that any multiple interruptions are not consecutive;
R⁵ is H; aryl; C₁-C₆ alkyl, straight, branched or cyclic, unsubstituted or substituted by halogen, alkoxy, cyano, hydroxyl, alkylamine or aryl, and may be interrupted by one or more of O, S or >NR⁶ provided that any multiple interruptions are not consecutive;
R⁶ is C₁-C₆ alkyl;
and salts or solvates thereof, with the proviso that the compound is not 2-(dimethylamino)ethyl 3,6-dichloro-2-methoxybenzoate or 2-(4-chlorophenoxy)acetic acid 2-(dimethylamino)ethyl ester.

2. A compound of Formula (A) characterised in that the compound is a compound of Formula (A1); 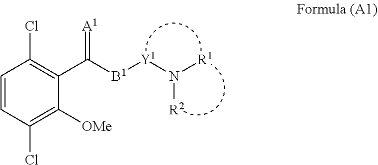
wherein;
dotted lines indicate that the indicated atoms may form part of a cyclic structure;
A¹ is O or S;
B¹ is O, S or N;
Y¹ is C₂-C₉ alkyl, straight, branched or cyclic, unsubstituted or substituted by halogen, alkoxy, cyano, hydroxyl, carbonyl, sulfonyl, sulfinyl, alkylamine or aryl, may be interrupted by O, S or >NR₃;
R¹ is H; aryl; C₁-C₆ alkyl, straight, branched or cyclic, unsubstituted or substituted by halogen, alkoxy, cyano, hydroxyl, carbonyl, sulfonyl, sulfinyl, alkylamine or aryl, and may be interrupted by one or more of O, S or >NR₃, provided that any multiple interruptions are not consecutive;
R³ is C₁-C₆ alkyl;
R² is H; aryl; C₁-C₆ alkyl, straight, branched or cyclic, unsubstituted or substituted by halogen, alkoxy, cyano, hydroxyl, carbonyl, sulfonyl, sulfinyl, alkylamine or aryl, and may be interrupted by one or more of O, S or >NR₃ provided that any multiple interruptions are not consecutive;
or R² is 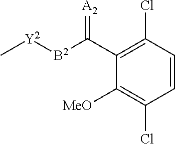
and wherein;
A² is O or S;
B² is O, S or N;
Y² is C₂-C₉ alkyl, straight, branched or cyclic, unsubstituted or substituted by halogen, alkoxy, cyano, hydroxyl, carbonyl, sulfonyl, sulfinyl, alkylamine or aryl, may be interrupted by one or more of O, S or >NR₃ provided that any multiple interruptions are not consecutive;
and salts or solvates thereof, with the proviso that the compound is not 2-(dimethylamino)ethyl 3,6-dichloro-2-methoxybenzoate.

3. An agricultural composition comprising an effective amount of a compound of Formula (B), including salts and solvates thereof, and an agriculturally acceptable adjuvant or carrier; 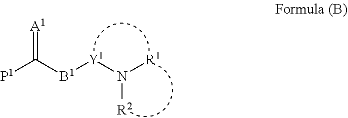
wherein;
dotted lines indicate that the indicated atoms may form part of a cyclic structure;
P¹ is selected from the group consisting of; 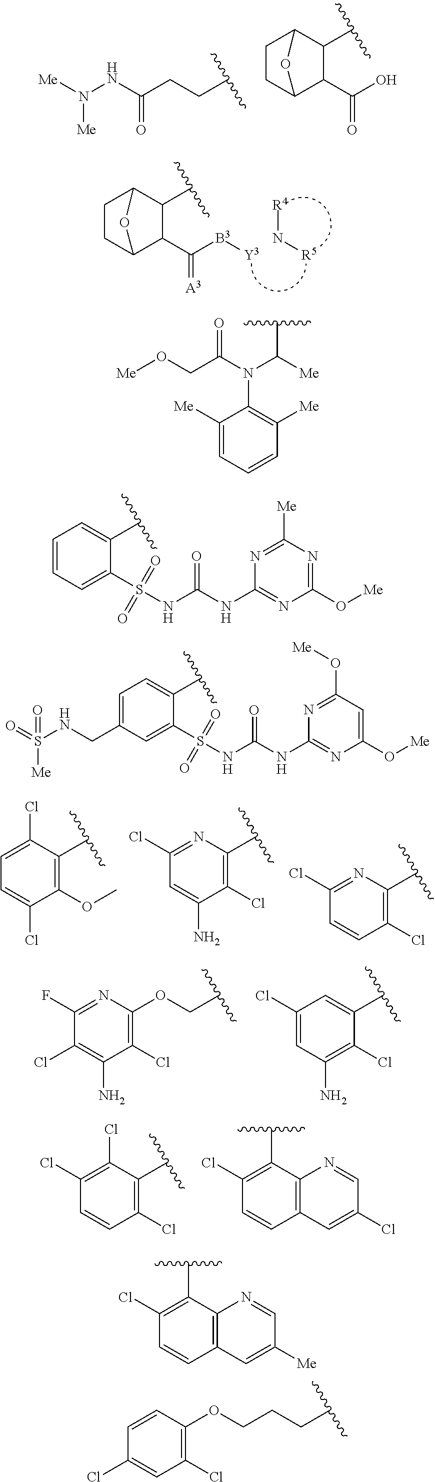 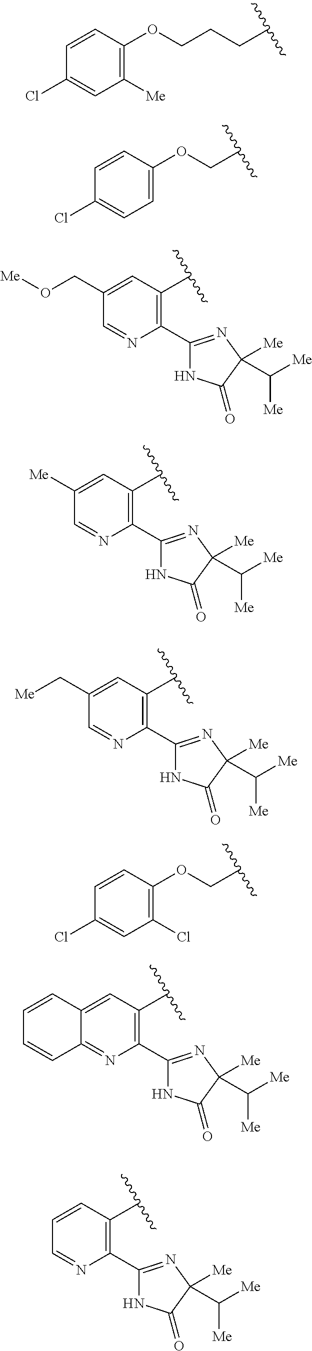 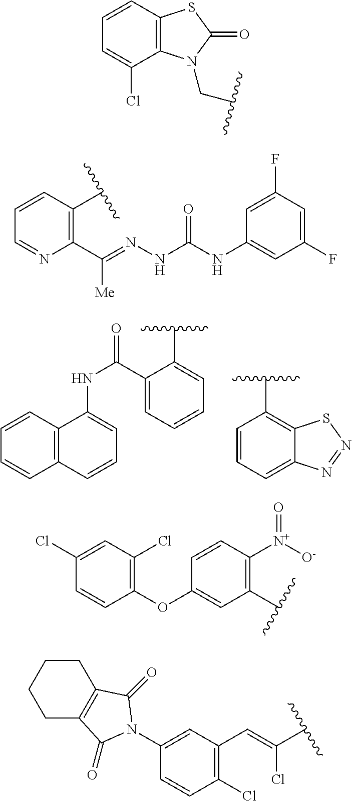
A¹ is O or S;
B¹ is O or S;
Y¹ is C₂-C₉ alkyl, straight, branched or cyclic, unsubstituted or substituted by halogen, alkoxy, cyano, hydroxyl, alkylamine or aryl, may be interrupted by O, S or >NR³;
R¹ is H; aryl; C₁-C₆ alkyl, straight, branched or cyclic, unsubstituted or substituted by halogen, alkoxy, cyano, hydroxyl, alkylamine or aryl, and may be interrupted by one or more of O, S or >NR₃, provided that any multiple interruptions are not consecutive;
R³ is C₁-C₆ alkyl;
R² is H; aryl; C₁-C₆ alkyl, straight, branched or cyclic, unsubstituted or substituted by halogen, alkoxy, cyano, hydroxyl, alkylamine or aryl, and may be interrupted by one or more of O, S or >NR³ provided that any multiple interruptions are not consecutive;
or R² is 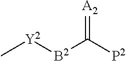
and wherein;
P² is, independently of P¹ selected from the group consisting of; 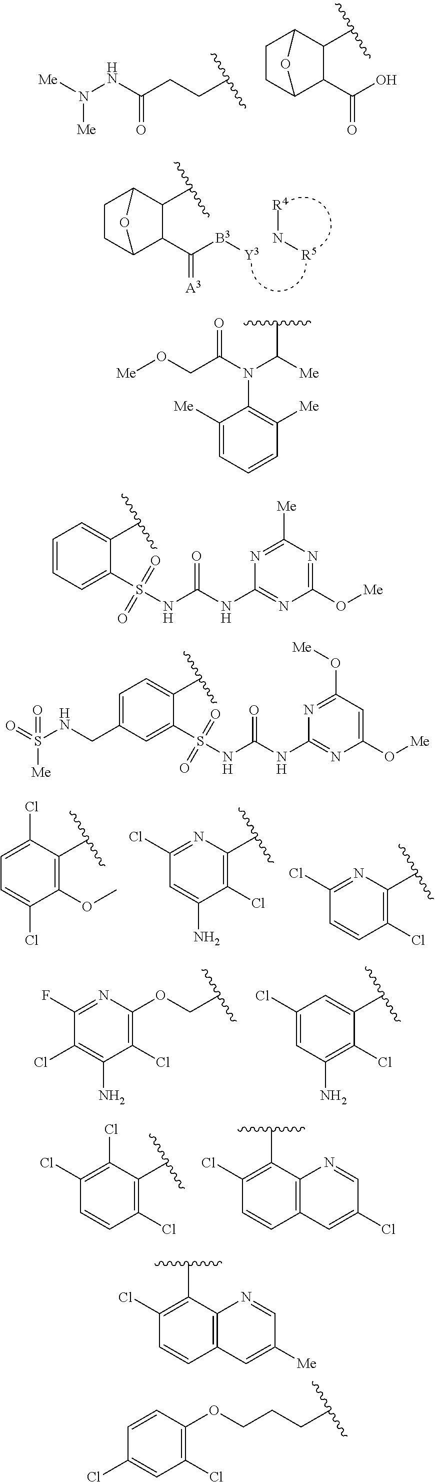 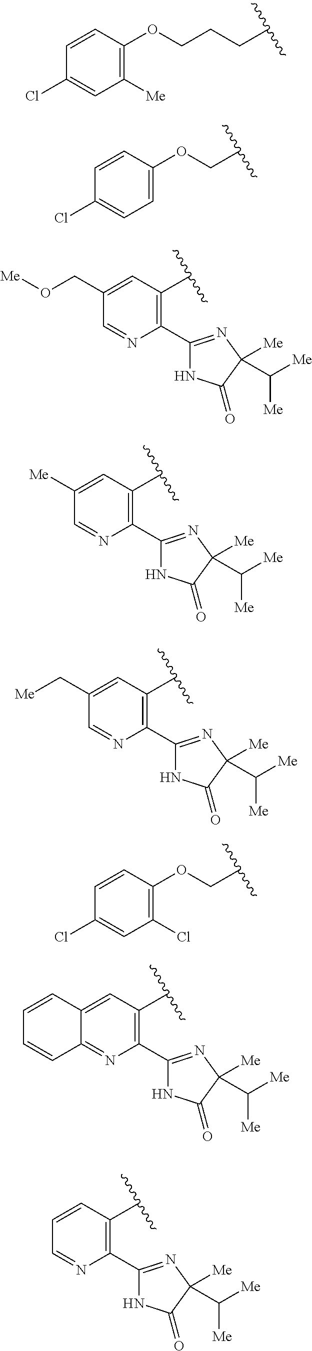 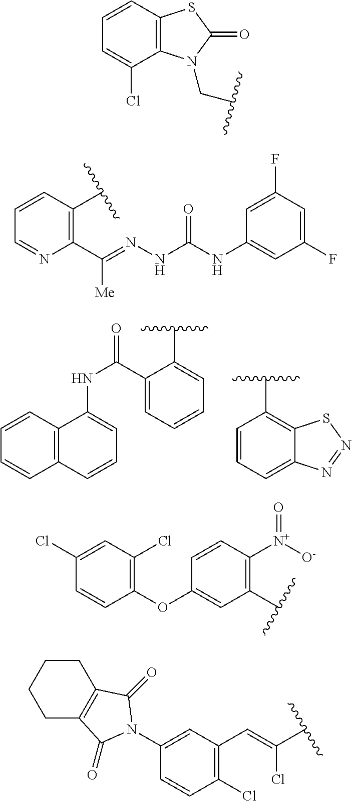
A² is O or S;
B² is O or S;
Y² is C₂-C₉ alkyl, straight, branched or cyclic, unsubstituted or substituted by halogen, alkoxy, cyano, hydroxyl, alkylamine or aryl, may be interrupted by one or more of O, S or >NR₃ provided that any multiple interruptions are not consecutive;
and wherein;
A³ is O or S;
B³ is O or S;
Y³ is C₂-C₉ alkyl, straight, branched or cyclic, unsubstituted or substituted by halogen, alkoxy, cyano, hydroxyl, alkylamine or aryl, may be interrupted by O, S or >NR⁶;
R⁴ is H; aryl; C₁-C₆ alkyl, straight, branched or cyclic, unsubstituted or substituted by halogen, alkoxy, cyano, hydroxyl, alkylamine or aryl, and may be interrupted by one or more of O, S or >NR₃, provided that any multiple interruptions are not consecutive;
R⁵ is H; aryl; C₁-C₆ alkyl, straight, branched or cyclic, unsubstituted or substituted by halogen, alkoxy, cyano, hydroxyl, alkylamine or aryl, and may be interrupted by one or more of O, S or >NR⁶ provided that any multiple interruptions are not consecutive;
R⁶ is C₁-C₆ alkyl.

4. An agricultural composition of claim 3 characterised in that the agricultural composition is a herbicidal composition comprising a herbicidally-effective amount of a compound of Formula (B1), or salts and solvates thereof, and an agriculturally acceptable adjuvant or carrier: 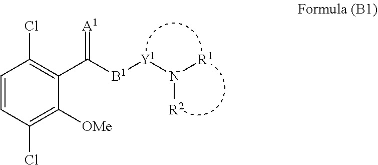
wherein;
dotted lines indicate that the indicated atoms may form part of a cyclic structure;
A¹ is O or S;
B¹ is O or S;
Y¹ is C₂-C₉ alkyl, straight, branched or cyclic, unsubstituted or substituted by halogen, alkoxy, cyano, hydroxyl, carbonyl, sulfonyl, sulfinyl, alkylamine or aryl, may be interrupted by O, S or >NR₃;
R¹ is H; aryl; C₁-C₆ alkyl, straight, branched or cyclic, unsubstituted or substituted by halogen, alkoxy, cyano, hydroxyl, carbonyl, sulfonyl, sulfinyl, alkylamine or aryl, and may be interrupted by one or more of O, S or >NR₃, provided that any multiple interruptions are not consecutive;
R³ is C₁-C₆ alkyl;
R² is H; aryl; C₁-C₆ alkyl, straight, branched or cyclic, unsubstituted or substituted by halogen, alkoxy, cyano, hydroxyl, carbonyl, sulfonyl, sulfinyl, alkylamine or aryl, and may be interrupted by one or more of O, S or >NR₃ provided that any multiple interruptions are not consecutive;
or R² is 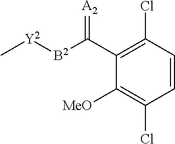
and wherein;
A² is O or S;
B² is O or S;
Y² is C₂-C₉ alkyl, straight, branched or cyclic, unsubstituted or substituted by halogen, alkoxy, cyano, hydroxyl, carbonyl, sulfonyl, sulfinyl, alkylamine or aryl, may be interrupted by one or more of O, S or >NR₃ provided that any multiple interruptions are not consecutive.

5. A herbicidal composition according to claim 4 characterised in that;
A¹ is O;
B¹ is O or S;
Y¹ is C₂-C₆ alkyl, straight, branched or cyclic, may be interrupted by O; and
R¹ and R² are, independently, C₁-C₆ alkyl, straight, branched or cyclic, may be interrupted by O.

6. A herbicidal composition according to claim 4 characterised in that, the compound of Formula (B1) is a compound selected from the group consisting of:

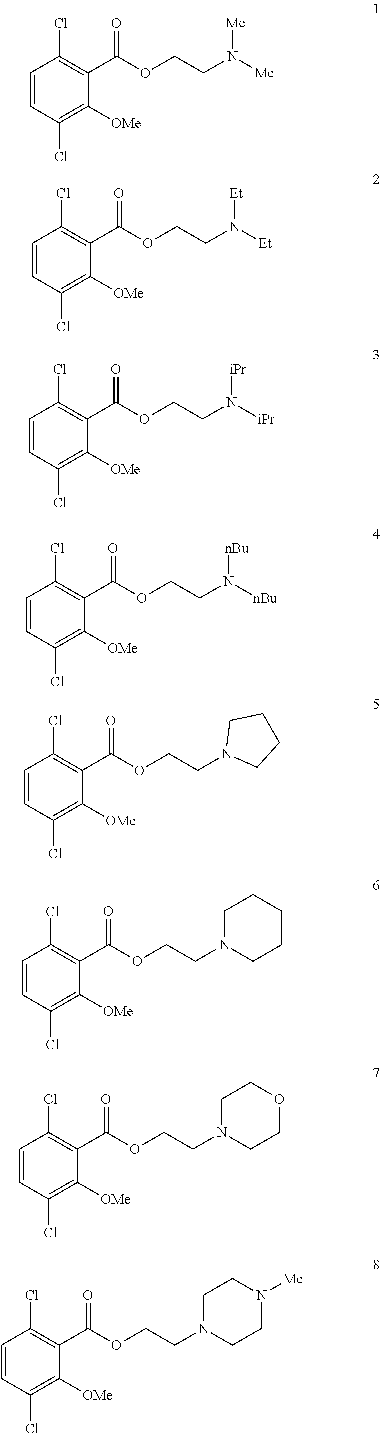

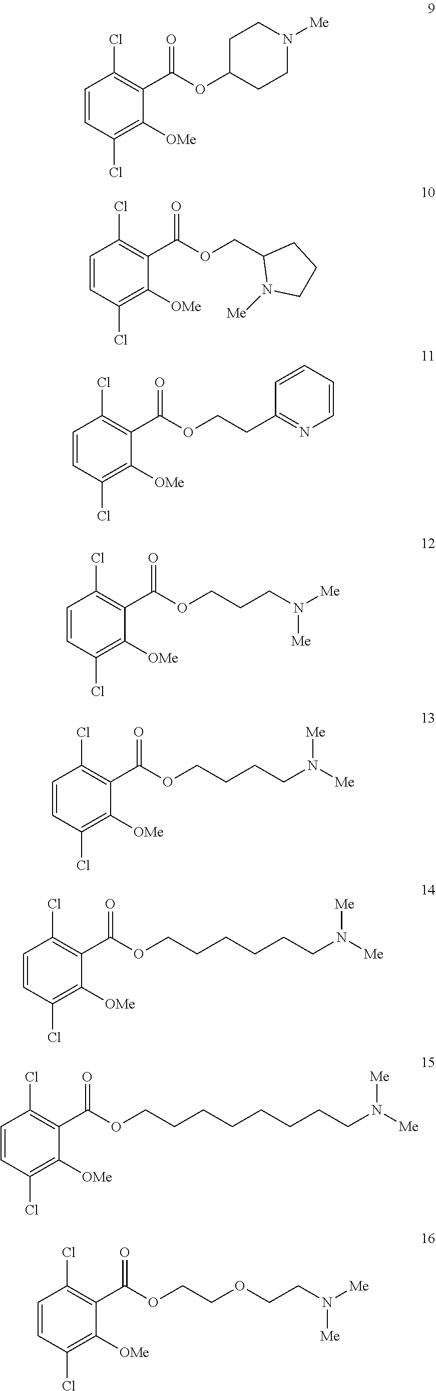

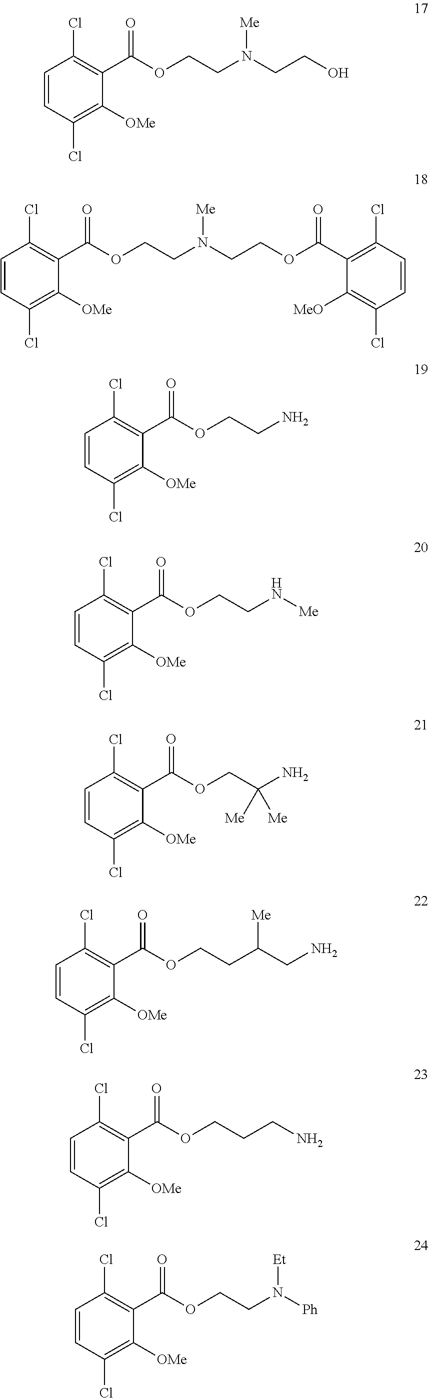

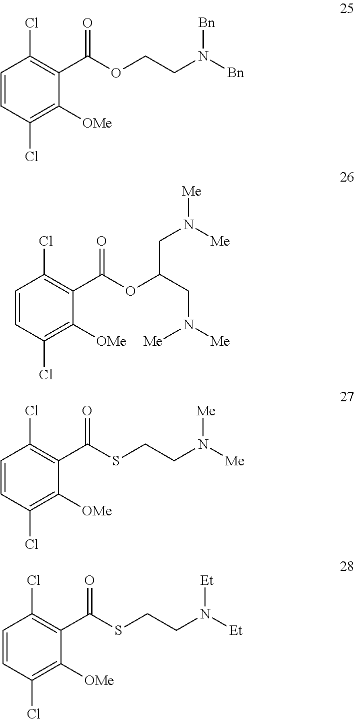

7. A herbicidal composition according to claim 6 characterised in that the compound of Formula (B1) is a compound selected from the group consisting of:

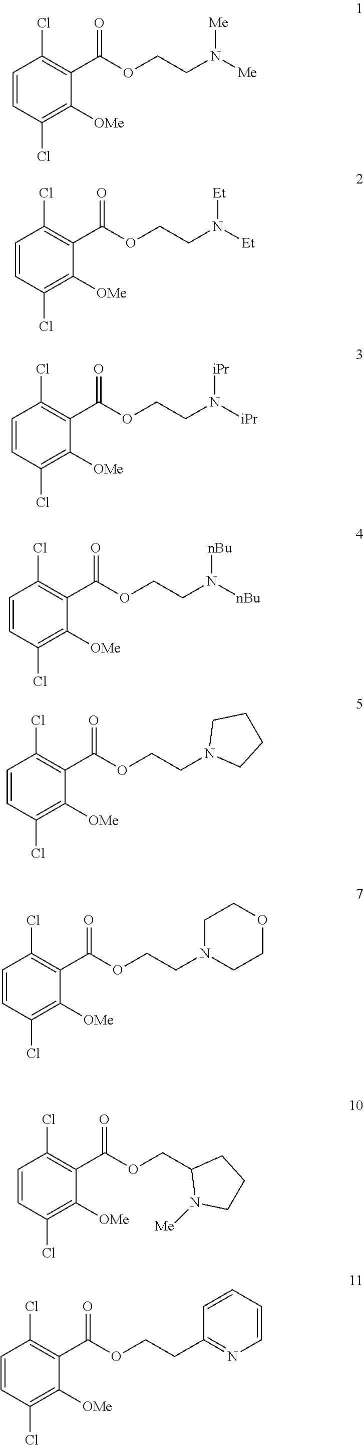

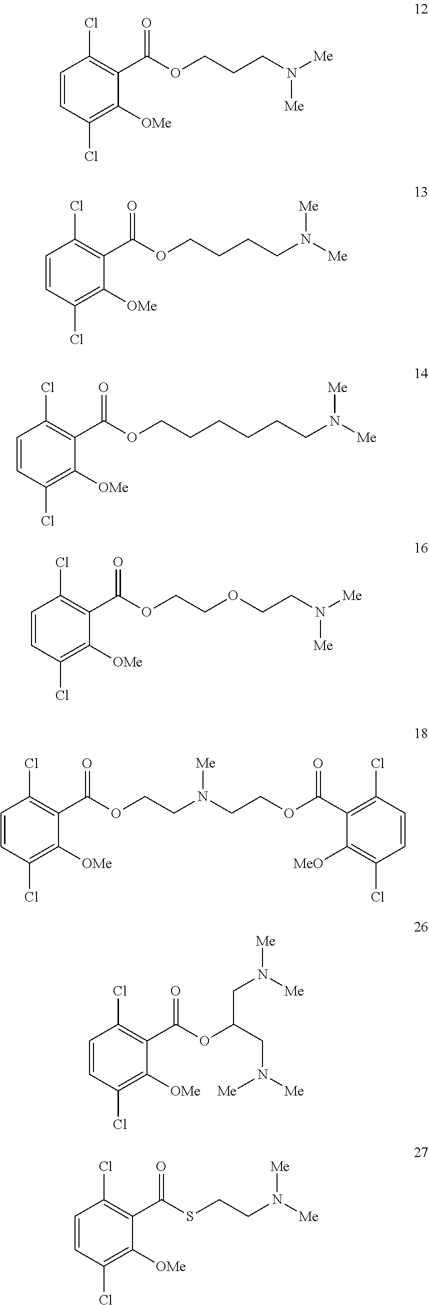

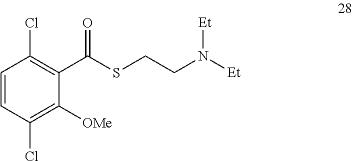 [SMILES: CCN(CC)CCSC(=O)c1c(Cl)ccc(Cl)c1OC]

8. A herbicidal composition according to claim 6 characterised in that the compound of Formula (B1) is a salt of the compound 2-(dimethylamino)ethyl 3,6-dichloro-2-methoxybenzoate.

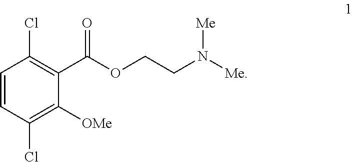 [SMILES: COc1c(Cl)ccc(Cl)c1C(=O)OCCN(C)C]

9. A herbicidal composition according to claim 8 characterised in that the herbicidal composition comprises the hydrochloride salt of 2-(dimethylamino)ethyl 3,6-dichloro-2-methoxybenzoate.

10. A method for use of an agricultural composition of claim 3, characterised in that the method comprises applying to a plant an effective amount of the agricultural composition.

11. A method for suppressing the growth of an auxin-susceptible plant, characterised in that the method comprises applying to the plant a herbicidally effective amount of a herbicidal composition of claim 4.

12. A method according to claim 11 characterised in that the auxin-susceptible plant is a weed in a crop.

13. A method according to claim 11 characterised in that the crop is a dicamba-resistant crop selected from the list consisting of: corn, cotton and soybean.

14. A method according to claim 11 characterised in that a crop plant of the crop is modified by introduction of the dmo gene from Stenotrophomonas maltophilia strain DI-6.
